SEQUENCE LISTING (53 sequences)

SEQ ID NO: 1 - protein, 69 aa, Escherichia coli
TPDCVTGKVEYTKYNDDDTFTVKVGDKELFTNRWNLQSLLLSAQITGMTVTIKTNACHNGGGFSEVIFR

SEQ ID NO: 2 - protein, 69 aa, Artificial Sequence
  Consensus
  MISC_FEATURE (1)..(1): Xaa is selected among T, A or S
  MISC_FEATURE (16)..(16): Xaa is selected among D, E or N
  MISC_FEATURE (17)..(17): Xaa is selected among D, E or N
  MISC_FEATURE (18)..(18): Xaa is selected among D, E or N
  MISC_FEATURE (21)..(21): Xaa is selected among T, A or S
  MISC_FEATURE (28)..(28): Xaa is selected among D, E or N
  MISC_FEATURE (29)..(29): Xaa is selected among L, I or V
  MISC_FEATURE (30)..(30): Xaa is selected among F, Y, W or A
  MISC_FEATURE (31)..(31): Xaa is selected among T, A or S
  MISC_FEATURE (32)..(32): Xaa is selected among N, E or S
  MISC_FEATURE (33)..(33): Xaa is selected among R, K or E
  MISC_FEATURE (34)..(34): Xaa is selected among W, F, Y or A
  MISC_FEATURE (35)..(35): Xaa is selected among D, E or N
  MISC_FEATURE (54)..(54): Xaa is selected among T, A or S
  MISC_FEATURE (55)..(55): Xaa is selected among N, E, D or S
  MISC_FEATURE (56)..(56): Xaa is selected among T, A or S
  MISC_FEATURE (60)..(60): Xaa is selected among G, A or S
  MISC_FEATURE (62)..(62): Xaa is selected among G, A, S or T
  MISC_FEATURE (63)..(63): Xaa is selected among F, L or Y
  MISC_FEATURE (64)..(64): Xaa is selected among T, A or S
XPDCVTGKVEYTKYNXXXTFXVKVGDKXXXXXXXXLQSLLLSAQITGMTVTIKXXXCHNXGXXXEVIFR

SEQ ID NO: 3 - protein, 69 aa, Artificial Sequence
  Clones
SPDCVTGKVEYTKYNNDDTFTVKVGDKELWTEKWNLQSLLLSAQITGMTVTIKSNACHNGGSFAEVIFR

SEQ ID NO: 4 - DNA, 207 bp, Artificial Sequence
  NA coding SEQ ID NO 3
tctcctgattgtgtaactggaaaggtggagtatacaaaatataataacgacgacacctttactgttaaagtgggtgataaagaactgtggactgaaaaatggaaccttcagtctcttcttctcagtgcgcaaattacggggatgactgtaaccattaaatctaacgcatgtcataatggtgggtcttttgcagaagttatttttcgt

SEQ ID NO: 5 - protein, 69 aa, Artificial Sequence; identical to SEQ ID NO: 3
  clones B12 - C03 - D12 - G05 - G11 - H11

SEQ ID NO: 6 - DNA, 207 bp, Artificial Sequence
  NA encoding SEQ ID NO 5
gcacctgattgtgtaactggaaaggtggagtatacaaaatataataacgacgacaccttttctgttaaagtgggtgataaagaactgtggactgaaaaatggaaccttcagtctcttcttctcagtgcgcaaattacggggatgactgtaaccattaaaactaacgcatgtcataatggtggggcactgtctgaagttatttttcgt

SEQ ID NO: 7 - protein, 69 aa, Artificial Sequence
  Clones A06 - C06
SPDCVTGKVEYTKYNNDDTFSVKVGDKEIYTSKWNLQSLLLSAQITGMTVTIKSNTCHNGGAFSEVIFR

SEQ ID NO: 8 - DNA, 207 bp, Artificial Sequence
  NA encoding SEQ ID NO 7
tctcctgattgtgtaactggaaaggtggagtatacaaaatataataacgacgacaccttttctgttaaagtgggtgataaagaaatctacacttctaaatggaaccttcagtctcttcttctcagtgcgcaaattacggggatgactgtaaccattaaatctaacacttgtcataatggtggggcattttctgaagttatttttcgt

SEQ ID NO: 9 - protein, 69 aa, Artificial Sequence
  clone B02
SPDCVTGKVEYTKYNDEDTFSVKVGDKEVWTNRCKLQSLLLSAQITGMTVTIKTSSCHNAGGLTEVIFR

SEQ ID NO: 10 - DNA, 207 bp, Artificial Sequence
  NA encoding SEQ ID NO 9
tctcctgattgtgtaactggaaaggtggagtatacaaaatataatgacgaagacaccttttctgttaaagtgggtgataaagaagtgtggactaaccgttgcaaacttcagtctcttcttctcagtgcgcaaattacggggatgactgtaaccattaaaacttcttcttgtcataatgcagggggtttgactgaagttatttttcgt

SEQ ID NO: 11 - protein, 69 aa, Artificial Sequence
  clone B05
APDCVTGKVEYTKYNDDNTFSVKVGDKELYTNRWNLQSLLLSAQITGMTVTIKTNSCHNGGGFAEVIFR

SEQ ID NO: 12 - DNA, 207 bp, Artificial Sequence
  NA encoding SEQ ID NO 11
gcacctgattgtgtaactggaaaggtggagtatacaaaatataatgacgacaacaccttttctgttaaagtgggtgataaagaactgtacactaaccgttggaaccttcagtctcttcttctcagtgcgcaaattacggggatgactgtaaccattaaaactaactcttgtcataatggtgggggttttgcagaagttatttttcgt

SEQ ID NO: 13 - protein, 20 aa, Artificial Sequence
  Signal peptide
MKKTLLIAASLSFFSASALA

SEQ ID NO: 14 - protein, 89 aa, Artificial Sequence
  Concatenation of SEQ ID NO 1 and 13
MKKTLLIAASLSFFSASALATPDCVTGKVEYTKYNDDDTFTVKVGDKELFTNRWNLQSLLLSAQITGMTVTIKTNACHNGGGFSEVIFR

SEQ ID NO: 15 - DNA, 60 bp, Artificial Sequence
  NA encoding SEQ ID NO 13
atgaaaaaaacattattaatagctgcatcgctttcatttttttcagcaagtgcgctggcg

SEQ ID NO: 16 - protein, 300 aa, Artificial Sequence
  Exemplary M13 pIII sequence
TVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAANGDA

SEQ ID NO: 17 - protein, 426 aa, Artificial Sequence
  Example of STxB - PIII fusion protein
MATPDCVTGKVEYTKYNDDDTFTVKVGDKELFTNRWNLQSLLLSAQITGMTVTIKTNACHNGGGFSEVIFRAAAHHHHHHGAAEQKLISEEDLNGAAEQKLISEEDLNGAAEQKLISEEDLNGAAQTVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAANGDA

SEQ ID NO: 18 - DNA, 1277 bp, Artificial Sequence
  NA encoding SEQ ID NO 17
atggcgacgcctgattgtgtaactggaaaggtggagtatacaaaatataatgatgacgatacctttacgttaaagtgggtgataaagaattatttaccaacagatggaatcttcagtctcttcttctcagtgcgcaaattacggggatgactgtaaccattaaaactaatgcctgtcataatggagggggattcagcgaagttatttttcgtgcggccgcacatcatcatcaccatcacggggccgcggaacaaaaactcatctcagaagaggatctgaatggggccgcagagcaaaagctaatatctgaagaagatctcaacggggccgcagaacagaaacttatcagtgaggaggacttgaatggggccgcatagactgttgaaagttgtttagcaaaacctcatacagaaaattcatttactaacgtctggaaagacgacaaaactctagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctaatggcgacgcc

SEQ ID NO: 19 - DNA, 903 bp, Artificial Sequence
  NA encoding pIII fragment
actgttgaaagttgtttagcaaaacctcatacagaaaattcatttactaacgtctggaaagacgacaaaactctagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctaatggcgacgcctga

SEQ ID NO: 20 - protein, 426 aa, Artificial Sequence
  A3 - D10 - H3 - pIII fusion
MASPDCVTGKVEYTKYNNDDTFTVKVGDKELWTEKWNLQSLLLSAQITGMTVTIKSNACHNGGSFAEVIFRAAAHHHHHHGAAEQKLISEEDLNGAAEQKLISEEDLNGAAEQKLISEEDLNGAAQTVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAANGDA

SEQ ID NO: 21 - DNA, 1281 bp, Artificial Sequence
  NA encoding A3 - D10 - H3 - pIII fusion
atggcgtctcctgattgtgtaactggaaaggtggagtatacaaaatataataacgacgacacctttactgttaaagtgggtgataaagaactgtggactgaaaaatggaaccttcagtctcttcttctcagtgcgcaaattacggggatgactgtaaccattaaatctaacgcatgtcataatggtgggtcttttgcagaagttatttttcgtgcggccgcacatcatcatcaccatcacggggccgcggaacaaaaactcatctcagaagaggatctgaatggggccgcagagcaaaagctaatatctgaagaagatctcaacggggccgcagaacagaaacttatcagtgaggaggacttgaatggggccgcatagactgttgaaagttgtttagcaaaacctcatacagaaaattcatttactaacgtctggaaagacgacaaaactctagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctaatggcgacgcctga

SEQ ID NO: 22 - protein, 426 aa, Artificial Sequence
  B12 - C03 - D12 - G05 - G11 - H11 - pIII fusion
MAAPDCVTGKVEYTKYNNDDTFSVKVGDKELWTEKWNLQSLLLSAQITGMTVTIKTNACHNGGALSEVIFRAAAHHHHHHGAAEQKLISEEDLNGAAEQKLISEEDLNGAAEQKLISEEDLNGAAQTVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAANGDA

SEQ ID NO: 23 - DNA, 1281 bp, Artificial Sequence
  NA encoding B12 - C03 - D12 - G05 - G11 - H11 - pIII fusion
atggcggcacctgattgtgtaactggaaaggtggagtatacaaaatataataacgacgacaccttttctgttaaagtgggtgataaagaactgtggactgaaaaatggaaccttcagtctcttcttctcagtgcgcaaattacggggatgactgtaaccattaaaactaacgcatgtcataatggtggggcactgtctgaagttatttttcgtgcggccgcacatcatcatcaccatcacggggccgcggaacaaaaactcatctcagaagaggatctgaatggggccgcagagcaaaagctaatatctgaagaagatctcaacggggccgcagaacagaaacttatcagtgaggaggacttgaatggggccgcatagactgttgaaagttgtttagcaaaacctcatacagaaaattcatttactaacgtctggaaagacgacaaaactctagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctaatggcgacgcctga

SEQ ID NO: 24 - protein, 426 aa, Artificial Sequence
  A06 - C06 - pIII fusion
MASPDCVTGKVEYTKYNNDDTFSVKVGDKEIYTSKWNLQSLLLSAQITGMTVTIKSNTCHNGGAFSEVIFRAAAHHHHHHGAAEQKLISEEDLNGAAEQKLISEEDLNGAAEQKLISEEDLNGAAQTVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAANGDA

SEQ ID NO: 25 - DNA, 1281 bp, Artificial Sequence
  NA encoding A06 - C06 - pIII fusion
atggcgtctcctgattgtgtaactggaaaggtggagtatacaaaatataataacgacgacaccttttctgttaaagtgggtgataaagaaatctacacttctaaatggaaccttcagtctcttcttctcagtgcgcaaattacggggatgactgtaaccattaaatctaacacttgtcataatggtggggcattttctgaagttatttttcgtgcggccgcacatcatcatcaccatcacggggccgcggaacaaaaactcatctcagaagaggatctgaatggggccgcagagcaaaagctaatatctgaagaagatctcaacggggccgcagaacagaaacttatcagtgaggaggacttgaatggggccgcatagactgttgaaagttgtttagcaaaacctcatacagaaaattcatttactaacgtctggaaagacgacaaaactctagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctaatggcgacgcctga

SEQ ID NO: 26 - protein, 426 aa, Artificial Sequence
  B02 - pIII fusion
MASPDCVTGKVEYTKYNDEDTFSVKVGDKEVWTNRCKLQSLLLSAQITGMTVTIKTSSCHNAGGLTEVIFRAAAHHHHHHGAAEQKLISEEDLNGAAEQKLISEEDLNGAAEQKLISEEDLNGAAQTVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAANGDA

SEQ ID NO: 27 - DNA, 1281 bp, Artificial Sequence
  NA encoding B02 - pIII fusion
atggcgtctcctgattgtgtaactggaaaggtggagtatacaaaatataatgacgaagacaccttttctgttaaagtgggtgataaagaagtgtggactaaccgttgcaaacttcagtctcttcttctcagtgcgcaaattacggggatgactgtaaccattaaaacttcttcttgtcataatgcagggggtttgactgaagttatttttcgtgcggccgcacatcatcatcaccatcacggggccgcggaacaaaaactcatctcagaagaggatctgaatggggccgcagagcaaaagctaatatctgaagaagatctcaacggggccgcagaacagaaacttatcagtgaggaggacttgaatggggccgcatagactgttgaaagttgtttagcaaaacctcatacagaaaattcatttactaacgtctggaaagacgacaaaactctagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctaatggcgacgcctga

SEQ ID NO: 28 - protein, 426 aa, Artificial Sequence
  B05 - pIII fusion
MAAPDCVTGKVEYTKYNDDNTFSVKVGDKELYTNRWNLQSLLLSAQITGMTVTIKTNSCHNGGGFAEVIFRAAAHHHHHHGAAEQKLISEEDLNGAAEQKLISEEDLNGAAEQKLISEEDLNGAAQTVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAANGDA

SEQ ID NO: 29 - DNA, 1281 bp, Artificial Sequence
  NA encoding B05 - pIII fusion
atggcggcacctgattgtgtaactggaaaggtggagtatacaaaatataatgacgacaacaccttttctgttaaagtgggtgataaagaactgtacactaaccgttggaaccttcagtctcttcttctcagtgcgcaaattacggggatgactgtaaccattaaaactaactcttgtcataatggtgggggttttgcagaagttatttttcgtgcggccgcacatcatcatcaccatcacggggccgcggaacaaaaactcatctcagaagaggatctgaatggggccgcagagcaaaagctaatatctgaagaagatctcaacggggccgcagaacagaaacttatcagtgaggaggacttgaatggggccgcatagactgttgaaagttgtttagcaaaacctcatacagaaaattcatttactaacgtctggaaagacgacaaaactctagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctaatggcgacgcctga

SEQ ID NO: 30 - DNA, 207 bp, Escherichia coli
acgcctgattgtgtaactggaaaggtggagtatacaaaatataatgatgacgatacctttacagttaaagtgggtgataaagaattatttaccaacagatggaatcttcagtctcttcttctcagtgcgcaaattacggggatgactgtaaccattaaaactaatgcctgtcataatggagggggattcagcgaagttatttttcgt

SEQ ID NO: 31 - DNA, 207 bp, Artificial Sequence
  nucleic acid sequence encoding STxB D18E, G62T mutant of SEQ ID NO 32
acgcctgattgtgtaactggaaaggtggagtatacaaaatataatgatgaagatacctttacagttaaagtgggtgataaagaattatttaccaacagatggaatcttcagtctcttcttctcagtgcgcaaattacggggatgactgtaaccattaaaactaatgcctgtcataatggagggacattcagcgaagttatttttcgt

SEQ ID NO: 32 - protein, 69 aa, Artificial Sequence
  STxB D18E, G62T mutant sequence
TPDCVTGKVEYTKYNDEDTFTVKVGDKELFTNRWNLQSLLLSAQITGMTVTIKTNACHNGGTFSEVIFR

SEQ ID NO: 33 - protein, 426 aa, Artificial Sequence
  STxB mutant of SEQ ID NO 31 fused to pIII
MATPDCVTGKVEYTKYNDEDTFTVKVGDKELFTNRWNLQSLLLSAQITGMTVTIKTNACHNGGTFSEVIFRAAAHHHHHHGAAEQKLISEEDLNGAAEQKLISEEDLNGAAEQKLISEEDLNGAAQTVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAANGDA

SEQ ID NO: 34 - DNA, 1278 bp, Artificial Sequence
  nucleic acid sequence encoding SEQ ID NO 33
atggcgacgcctgattgtgtaactggaaaggtggagtatacaaaatataatgatgaagatacctttacagttaaagtgggtgataaagaattatttaccaacagatggaatcttcagtctcttcttctcagtgcgcaaattacggggatgactgtaaccattaaaactaatgcctgtcataatggagggacattcagcgaagttatttttcgtgcggccgcacatcatcatcaccatcacggggccgcggaacaaaaactcatctcagaagaggatctgaatggggccgcagagcaaaagctaatatctgaagaagatctcaacggggccgcagaacagaaacttatcagtgaggaggacttgaatggggccgcatagactgttgaaagttgtttagcaaaacctcatacagaaaattcatttactaacgtctggaaagacgacaaaactctagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctaatggcgacgcc

SEQ ID NO: 35 - DNA, 4804 bp, Artificial Sequence
  pHEN2_STxB phagemid
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtccacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtccacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaaattctatttcaaggagacagtcataatgaaatacctattgcctacggcagccgctggattgttattactcgcggcccagccggccatggcgacgcctgattgtgtaactggaaaggtggagtatacaaaatataatgatgacgatacctttacagttaaagtgggtgataaagaattatttaccaacagatggaatcttcagtctcttcttctcagtgcgcaaattacggggatgactgtaaccattaaaactaatgcctgtcataatggagggggattcagcgaagttatttttcgtgcggccgcacatcatcatcaccatcacggggccgcggaacaaaaactcatctcagaagaggatctgaatggggccgcagagcaaaagctaatatctgaagaagatctcaacggggccgcagaacagaaacttatcagtgaggaggacttgaatggggccgcatagactgttgaaagttgtttagcaaaacctcatacagaaaattcatttactaacgtctggaaagacgacaaaactctagatcgttacgctaactatgagggctgtctgtggaatgctacaggcgttgtggtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggtgcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatataaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgggtccactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 36 - protein, 69 aa, Artificial Sequence
  SEQ ID NO 1 with the first amino-acid residue being A
APDCVTGKVEYTKYNDDDTFTVKVGDKELFTNRWNLQSLLLSAQITGMTVTIKTNACHNGGGFSEVIFR

SEQ ID NO: 37 - protein, 69 aa, Artificial Sequence
  Consensus
  MISC_FEATURE (1)..(1): Xaa is selected among T, A or S
  MISC_FEATURE (16)..(16): Xaa is selected among D, E or N
  MISC_FEATURE (21)..(21): Xaa is selected among T, A or S
  MISC_FEATURE (29)..(29): Xaa is selected among L, I or V
  MISC_FEATURE (30)..(30): Xaa is selected among F, Y, W or A
  MISC_FEATURE (32)..(32): Xaa is selected among N, E or S
  MISC_FEATURE (33)..(33): Xaa is selected among R, K or E
  MISC_FEATURE (54)..(54): Xaa is selected among T, A or S
  MISC_FEATURE (56)..(56): Xaa is selected among T, A or S
  MISC_FEATURE (62)..(62): Xaa is selected among G, A, S or T
  MISC_FEATURE (63)..(63): Xaa is selected among F, L or Y
  MISC_FEATURE (64)..(64): Xaa is selected among T, A or S
XPDCVTGKVEYTKYNXDDTFXVKVGDKEXXTXXWNLQSLLLSAQITGMTVTIKXNXCHNGGXXXEVIFR

SEQ ID NO: 38 - protein, 14 aa, Artificial Sequence
  S1
PDCVTGKVEYTKYN

SEQ ID NO: 39 - protein, 6 aa, Artificial Sequence
  S3
VKVGDK

SEQ ID NO: 40 - protein, 18 aa, Artificial Sequence
  S4
LQSLLLSAQITGMTVTIK

SEQ ID NO: 41 - protein, 5 aa, Artificial Sequence
  S7
EVIFR

SEQ ID NO: 42 - protein, 424 aa, Unknown
  wild-type pIII protein having 424 amino-acids residues
MKKLLFAIPLVVPFYSHSAETVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 43 - protein, 69 aa, Artificial Sequence
  D16N mutant
TPDCVTGKVEYTKYNNDDTFTVKVGDKELFTNRWNLQSLLLSAQITGMTVTIKTNACHNGGGFSEVIFR

SEQ ID NO: 44 - protein, 69 aa, Artificial Sequence
  D17N mutant
TPDCVTGKVEYTKYNDNDTFTVKVGDKELFTNRWNLQSLLLSAQITGMTVTIKTNACHNGGGFSEVIFR

SEQ ID NO: 45 - protein, 69 aa, Artificial Sequence
  D17E mutant
TPDCVTGKVEYTKYNDEDTFTVKVGDKELFTNRWNLQSLLLSAQITGMTVTIKTNACHNGGGFSEVIFR

SEQ ID NO: 46 - protein, 69 aa, Artificial Sequence
  D16N D17N mutant
TPDCVTGKVEYTKYNNNDTFTVKVGDKELFTNRWNLQSLLLSAQITGMTVTIKTNACHNGGGFSEVIFR

SEQ ID NO: 47 - protein, 69 aa, Artificial Sequence
  D18N mutant
TPDCVTGKVEYTKYNDDNTFTVKVGDKELFTNRWNLQSLLLSAQITGMTVTIKTNACHNGGGFSEVIFR

SEQ ID NO: 48 - protein, 69 aa, Artificial Sequence
  F30A mutant
TPDCVTGKVEYTKYNDDDTFTVKVGDKELATNRWNLQSLLLSAQITGMTVTIKTNACHNGGGFSEVIFR

SEQ ID NO: 49 - protein, 69 aa, Artificial Sequence
  R33D mutant
TPDCVTGKVEYTKYNDDDTFTVKVGDKELFTNDWNLQSLLLSAQITGMTVTIKTNACHNGGGFSEVIFR

SEQ ID NO: 50 - protein, 69 aa, Artificial Sequence
  W34G mutant
TPDCVTGKVEYTKYNDDDTFTVKVGDKELFTNRGNLQSLLLSAQITGMTVTIKTNACHNGGGFSEVIFR

SEQ ID NO: 51 - protein, 69 aa, Artificial Sequence
  W34A mutant
TPDCVTGKVEYTKYNDDDTFTVKVGDKELFTNRANLQSLLLSAQITGMTVTIKTNACHNGGGFSEVIFR

SEQ ID NO: 52 - protein, 69 aa, Artificial Sequence
  G62T mutant
TPDCVTGKVEYTKYNDDDTFTVKVGDKELFTNRWNLQSLLLSAQITGMTVTIKTNACHNGGTFSEVIFR

SEQ ID NO: 53 - protein, 69 aa, Artificial Sequence; identical to SEQ ID NO: 32
  D17E G62T mutant

(19) United States
(12) Patent Application Publication
(10) Pub. No.: US 2021/0107951 A1
(43) Pub. Date: Apr. 15, 2021

(54) PEPTIDES, ESPECIALLY POLYPEPTIDES, PHAGE DISPLAY SCREENING METHOD AND ASSOCIATED MEANS, AND THEIR USES FOR RESEARCH AND BIOMEDICAL APPLICATIONS

(71) Applicants: INSTITUT NATIONAL DE LA SANTE ET DE LA RECHERCHE MEDICALE, PARIS CEDEX13 (FR); CENTRE NATIONAL DE LA RECHERCHE SCIENTIFIQUE, PARIS CEDEX 16 (FR); INSTITUT CURIE, PARIS (FR); UNIVERSITE' PARIS-SACLAY, Saint Aubin (FR)

(72) Inventors: Thomas MURARASU, PARIS (FR); Franck PEREZ, PARIS (FR); Ludger JOHANNES, COURBEVOIE (FR)

(21) Appl. No.: 16/606,494

(22) PCT Filed: Mar. 13, 2018
(86) PCT No.: PCT/EP2018/056311

§ 371 Date: Oct. 18, 2019

(30) Foreign Application Priority Data
Apr. 20, 2017 (EP) 17305454.5

(51) Int. Cl. C07K 14/25; C07K 14/005

(52) U.S. Cl. CPC C07K 14/25; C07K 2319/735; C07K 2319/01; C07K 14/005

(57) ABSTRACT

Disclosed are peptides, hosts expressing such peptides and a process for producing and screening such peptides or hosts. Also disclosed is use of the peptide or host expressing such peptide in the detection of disease, to a method for constructing a library of hosts, in particular of phages expressing peptides. Also disclosed is a library of hosts expressing peptides and its use for example for detecting molecules and/or cells in a sample, in the treatment of disease. These find application in the therapeutic and diagnostic medical technical fields.

### BACKGROUND OF THE INVENTION

### Field of the Invention

The invention relates to peptides, hosts expressing such peptides and a process for producing and screening such peptides or hosts. The invention also relates to use of the peptide or host expressing such peptide in the detection of disease.

The invention also relates to a method for constructing a library of hosts, in particular of phages displaying peptides.

The invention also concerns the field of filamentous bacteriophages, and in particular relates to particularly designed filamentous bacteriophages displaying at least one and up to five STxB-subunit(s) or variant(s) thereof at their surface. Accordingly, the invention also relates to a library of hosts expressing peptides and its use for example for detecting molecules and/or cells in a sample, in the treatment of disease.

The invention also relates to nucleic acid molecules suitable for enabling the production of such phages, including vectors, especially plasmids, more particularly phagemids or phage vectors. Bacterial cells, libraries, and methods of production of filamentous phage(s), or libraries thereof, are also part of the invention.

A particular aspect of the invention concerns a method of identifying one or more filamentous phage(s) displaying an (i.e., at least one) STxB-subunit or a variant thereof, which bind, in particular specifically bind, to a particular glycosphingolipid or a variant thereof, or to a mixture of several glycosphingolipids or variants thereof as a target.

The invention accordingly also pertains to the field of screening methods for new therapeutically active tools, more particularly to the phage display technique adapted to particular proteins, for research and medical applications. The invention simultaneously also relates to the field of screening methods enabling the determination of new therapeutic targets. Means enabling such achievements are also part of the present invention.

The invention finally also concerns filamentous bacteriophage of the invention, for use as a medicament, and use of labelled filamentous bacteriophage as a probe or marker for in vitro detection of glycosphingolipid(s) or variant(s) thereof.

The present invention finds application in the therapeutic field, let it be in for the treatment or for diagnostic purposes.

In the description below, the reference between square brackets ([ ]) refer to the list of references given at the end of the text.

### Description of the Related Art

Cancer remains the most common malignancy and second-most common cause of death in the Western world. Early detection is essential for curative cancer therapy and for achieving a decrease in cancer mortality.

Many different types of processes and techniques are used for detecting cancer. The processes/techniques used for detecting cancer are dependent on many parameters such as the person's age, medical condition, the type of cancer suspected, the severity of the symptoms, and previous test results. Common processes include for example, for most cancers: taking a biopsy which clearly determines the cancer diagnosis, magnetic resonance imaging (MRI), sonography or ultrasonography to reveal tumors in a tissue. When cancers of the rectum, colon, and uterus are targeted: using barium as a contrast medium and X rays to reveal abnormalities, digital rectal exam (DRE); or sigmoidoscopy. When bone, bone marrow or blood cells are targeted: bone marrow aspiration and biopsy, bone scanning, computed tomography (CT) scanning. When the digestive system is targeted: endoscopy, colonoscopy, upper endoscopy; and for example when the breast is targeted: breast MRI and mammography.

Other techniques are based on substances being found at higher than normal levels in the blood, urine, or body tissue of people with cancer. These techniques use “biomarkers” which can be correlated with the presence or absence of cancer.

However, the techniques/processes used, except biopsy, alone cannot clearly and specifically directly determine the type of cancer and need to be confirmed by using other methods in combination. In particular, biomarkers alone are often not sufficient to diagnose cancer. For example, in the case of acute leukemia, the complete blood count (CBC) cannot be particularly accurately measured, which renders the diagnosis very difficult to be established, and only a bone marrow biopsy allows diagnosis. Thus, the time taken to clearly determine the cancer and/or disease is increased due to the number of tests to be carried out. Since early detection is essential for curative cancer therapy and for achieving a decrease in cancer mortality there is a real need to find methods and/or compounds, that allow more efficient and/or precise diagnosis of cancer.

In the same way as many other diseases, there is a need to find better or faster methods of diagnosis.

There is therefore a real need to find methods and/or compounds, which allow more efficient and/or precise diagnosis and/or treatment of cancer. In particular, there is a real need to find methods and/or compounds that allow earlier detection and/or determination of diseases and/or therapeutic targeting.

Once the cancer is detected, there is also a clear need to find better or faster treatment and/or means for improving the known treatment. Treatment methods include, for example, surgery to eliminate if possible the tumors with chemotherapy and/or radiation therapy, immunotherapy, targeted therapy, or hormone therapy. It may also comprise, for example in the case of leukemia, chemotherapy and/or radiation therapy and/or if necessary and possible bone marrow transplantation.

The most common drugs used in the treatment of cancer are cytotoxic drugs, which act by killing or preventing cell division. However, such drugs have problematic side effects since they damage noncancerous tissues or organs with a high proportion of actively dividing cells for example, bone marrow, hair follicles, gastro-intestinal tract, thereby limiting the acceptable amount and frequency of drug administration. In addition, the side effects of cytotoxic drugs could also influence and/or reduce the compliance of the treatment of patients. Accordingly, an important improvement in the treatment of cancer is to find drugs more specific to the tumor cells and/or or means that allow to target cytotoxic compounds and/or drugs to tumor cells.

There is therefore a real need to find means and/or drugs that specifically target tumor cells and/or improve the delivery of cytotoxic compounds to tumor cells to reduce/eliminate the known side effects of cytotoxic compounds.

Several means are already known or used for targeting compounds to particular tissue and/or cells. In most cases, the target is a protein and/or other molecules expressed by the tissue and/or cell. For example, drugs coupled to monoclonal antibodies are used for targeting drugs to cells and/or tissue expressing the corresponding antigen. The antigen is often a biomarker or biomarkers of the disease. However, there is a binding limit of the monoclonal antibodies, the specificity and the selectivity of monoclonal antibodies may vary with regards to the antigen, such as glycosphingolipids.

There is therefore a real need to find means and/or drugs which allow a better targeting and/or allow to target particular biomarkers such as such as glycosphingolipids to improve the delivery of cytotoxic compounds to tumor cells to reduce/eliminate the known side effects of cytotoxic compounds.

Proteins pertaining to the Shiga toxin family are known to target glycosphingolipids.

Shiga toxin family members, i.e., so-called Shiga and Shiga-like toxins herein, are produced by Shigella dysenteriae and enterohemorrhagic (EHEC) strains of Escherichia coli. These toxins are composed of two non-covalently attached parts: the enzymatically active A-subunit, and the non-toxic, pentameric B-subunit (STxB). Shiga toxin family members encompass structurally and functionally related exotoxins, which include Shiga toxin from Shigella dysenteriae serotype 1 and the Shiga toxins that are produced by enterohemorrhagic strains of Escherichia coli, as detailed in particular in Johannes and Römer, Nat Rev Microbiol. 2010 February; 8(2):105-16. doi: 10.1038/nrmicro2279. Epub 2009 Dec. 21. This publication provides a description of known Stx1 and Stx2 variants. In particular, Table 1 of Johannes and Römer, 2010 provides a comparison of sequence similarity between Shiga toxin and EHEC produced Shiga-like toxins, which encompass for example Stx1 and Stx2 variants. Of note, Stx2 variants are 84-99% homologous to Stx2 but theses toxins only share at most 53% identity with the Shiga toxin. Present invention is by contrast construed around the so-called STx1B variant (as identified by NCBI reference sequence GenBank: ABR10023.1), taken as a reference sequence. It is observed that the STx1B variant of this database entry encompasses a peptide signal fused at its N-terminal extremity (referred to under SEQ ID NO: 13 herein).

Shiga toxin family members have an AB₅ molecular configuration. An enzymatically active monomeric A-subunit, STxA (which has a molecular mass of 32 kDa) is non-covalently associated with a pentamer of identical B fragments (each B fragment has a molecular mass of 7.7 kDa), also termed monomers herein, that form the B-subunit, STxB, the latter of which is responsible, in a pentameric form, for binding to cell surface receptors. STxB forms a doughnut-shaped structure with a central pore into which the carboxyl terminus of STxA inserts.

The first identified STxB moiety, as reported in N. A. Stockbine, M. P. Jackson, L. M. Sung, R. K. Holmes, A. D. O'Brien, J Bacteriol 170, 1116-22 (1988), specifically binds to the sugar moiety of the glycosphingolipid globotriaosylceramide (also known under the names CD77, Gb3, and ceramide trihexoside) found on the plasma membrane of target cells, which mediates uptake and intracellular transport of the toxin upon binding. Shiga toxin is internalized by clathrin-dependent and independent endocytosis, and is then transported to the endoplasmic reticulum following the retrograde route.

Bray et al., Current Biology Vol 11 No 9, 697-701, 2001 reports that the B-subunit of Shiga-like toxin 1 (SLT-1) is a small protein composed of 69 amino acid residues that pentamerizes spontaneously in solution. It has been proposed by Bray et al. to create combinatorial libraries of toxin variants with altered receptor specificity to identify toxin mutants able to kill cell lines resistant to the wild-type toxin. However, although Bray et al. successfully isolated a STxB variant, which was no more specific to Gb3, they could not identify its target. It is observed that the authors of Bray et al. produced a degenerate SLT-1 library by mutating only 9 amino acids positions with respect to the departure sequence: this does not encompass all possibilities of variability of a variant SLT-1 sequence while retaining functional properties of the same, as further assessed by the inventors of present invention. The present invention also further defines and refines this possible variability. It is also observed that the authors of Bray et al. necessarily mutated amino acids found in positions 15 and/or 19 of SLT-1 taken as a reference sequence.

Ling et al., 1998, Structure of the Shiga-like toxin I B-pentamer complexed with an analogue of its receptor Gb3. Biochemistry, 37(7), 1777-1788 observed that particular mutations of the Gb3 binding sites of the STxB moiety may abolish or considerably decrease the affinity of STxB for this GSL, without perturbing the tridimensional structure of the protein. There may thus be a certain degree of flexibility in the structure of STxB, which might allow for slight modifications of the binding site sequence.

Most B-subunits of Shiga toxin family members (STxB), let they be related to Stx1 or Stx2, are known to specifically bind the glycosphingolipid Gb3, which is a particular type of lipid that has been proven to be overexpressed on certain tumor cells, to the exception of the so-called SLT-Ile B moiety, which is a natural variant of the STxB family that binds preferentially Gb4 (see also Johannes et al. 2010 cited above).

Two studies have shown that it is possible to change of specificity of the SLT-IIe B scaffold. Ling et al., 2000, A mutant Shiga-like toxin Ile bound to its receptor Gb3 structure of a group II Shiga-like toxin with altered binding specificity. Structure, 3, 253-264, reported successful mutation of the SLT-IIe B variant in a way such that it changes its specificity from Gb4 to Gb3. Also, Boyd et al., 1993, Alteration of the glycolipid binding specificity of the pig edema toxin from globotetraosyl to globotriaosyl ceramide alters in vivo tissue targeting and results in a verotoxin 1-like disease in pigs. The Journal of Experimental Medicine, 177(6), 1745-1753, used site-directed mutagenesis specifically at positions Gln64 and Lys66 of the SLT-Ile B variant to convert the GSL binding specificity from Gb4 to Gb3. Previously, the authors of Tyrrell et al., PNAS USA 89 pp 524-528 (“Alteration of the carbohydrate binding specificity of verotoxins from Galalpha1-4Gal to GalNAcbeta1-3Galalpha1-4Gal and vice versa by site-directed mutagenesis of the binding subunit”) described a Gb3 specific variant that was mutated towards Gb4 specificity.

However, the prior art is silent with respect to how devise mutated STxB sequences in which all and only the positions involved in the binding sites are modified, with a remaining scaffold that allows proper maintenance of the structure and the oligomerization properties of such STxB monomers.

The prior art is also silent with respect to how devise mutated STxB sequences enabling further production of pentameric structures retaining pertinent functional properties, as discussed herein (binding properties, affinity or internalization properties, etc.).

A need therefore remains in defining possible positions of STxB that can actually be mutated, potentially conferring a new target specificity, meaning creation of new binding sites cavities in which the chemical environment is changed compare to wild type in order to engage new interaction with other carbohydrates.

Since Gb3 is a type of lipid that has been proven to be overexpressed on certain tumor cells, the use of STxB as a vector for tumor targeting has accordingly been proposed, as for instance disclosed in WO 02/060937 and WO 2004/016148. As reported in these disclosures, use of a so-called STxB vector as an universal carrier, i.e., when STxB is coupled to an antigen or an active ingredient, has the advantage that said vector and coupled active moiety can be internalized into Gb3 presenting cells, which is of great interest for the specific intracellular delivery of cytotoxic compounds (Johannes & W. Römer, Nature Reviews Microbiology 8, 105-116, 2010).

Chemical coupling of STxB to a number of cytotoxic compounds (such as the topoisomerase I inhibitor SN38 (El Alaoui et al., 2007 Shiga toxin-mediated retrograde delivery of a topoisomerase I inhibitor prodrug. Angewandte Chemie—International Edition, 46(34), 6469-6472), the benzodiazepine R05-4864 (El Alaoui et al., 2008 Synthesis and properties of a mitochondrial peripheral benzodiazepine receptor conjugate. ChemMedChem, 3(11), 1687-1695), and highly potent auristatin derivatives (Batisse et al., 2015 A new delivery system for auristatin in STxB-drug conjugate therapy. European Journal of Medicinal Chemistry, 95, 483-491) has been achieved. In a transgenic mouse model, it was shown that STxB targets Gb3-expressing spontaneous adeno-carcinomas of the gut following oral uptake or intravenous injection (Janssen et al., 2006 In vivo tumor targeting using a novel intestinal pathogen-based delivery approach. Cancer Research, 66(14), 7230-7236). The concept of using STxB as a delivery tool was then extended to human colorectal carcinoma. Primary cultures of tumoral enterocytes from surgical samples are targeted by STxB (Falguieres et al., 2008 Human colorectal tumors and metastases express Gb3 and can be targeted by an intestinal pathogen-based delivery tool. Molecular Cancer Therapeutics, 7(8), 2498-2508), and the protein is also efficiently taken up by xenografts of primary human tumors in mice (Viel et al., 2008 In vivo tumor targeting by the B-subunit of shiga toxin. Molecular Imaging, 7(6), 239-47). However, in mice, no therapeutic responses were obtained with the above-mentioned conjugates. Since Gb3 is also largely expressed by the kidney, a likely explanation is that therapeutic effects could not be reached due to dose limiting cytotoxicity of the vector.

Indeed, Gb3 is, unfortunately, also highly expressed on healthy tissues, particularly in the kidney, which considerably increases the risk of side effects, when treating patients with STxB-drug conjugates. On the other hand, other species of glycosphingolipid (such as so-called Gb4, Forsmann like iGb4, fucosyl-GM1, GM1, GM2, GD2, Globo-H, NeuAc-GM3, NeuGc-GM3, GD1a, O-acetyl-GD3, O-acteyl-GD2, O-acetyl-GT3, GD3, to cite a few) have been identified as promising targets for cancer therapy.

Based on these facts, there is undoubtedly a need for the development of research tools enabling the identification of particular, distinct and/or variant species of glycosphingolipids, especially distinct from Gb3, that can be found expressed in particular cancers or cancerous tissues, in particular with a certain specificity.

In this respect, it is another object of the present invention to devise methods and tools enabling highly specific and pertinent targeting of tumor cells with new therapeutic tools, according to potentially diverse tumors types and profiles.

This is why the inventors proposed to engineer the STxB scaffold, through a refined and cautious definition of its essence, in order to benefit from its potential as a delivery tool, taking into account the view to derive its specificity towards truly tumor-specific GSLs.

The well-established phage display technology allows to screen for protein candidates that are displayed on a bacteriophage. This displaying on phages enables the selection and the isolation of protein candidates, which potentially have a high affinity for a specific target, from a large library of mutants. Phage display has been greatly optimized and has been successfully used for the selection of antibodies, small antibodies (scFv, VHH nanobodies), and other libraries of non-antibody based scaffold (see T. Hey et al., Trends in Biotechnology, 2005), among which some have reached the clinics.

However, one can note that obtaining antibodies or other proteins with a high affinity for glycosphingolipids, whether it may be Gb3 or another glycosphingolipid, has remained a challenge.

Prior to the experiments reported herein, the inventors of the present invention experiences failed attempts in selecting Gb3 specific nanobodies by phage display selection, in particular using Gb3⁺ cell lines (Gb3 positive cell lines, displaying Gb3 at their surface). In fact, after three rounds of selection, the selected phages were not specific to glycosphingolipids. This also further emphasize the need for an alternative selection strategy for obtaining adequate tools enabling glycosphingolipid species identification, and the difficulties associated in defining them.

Turning now to the structural particularities of Shiga toxins, all Shiga toxin family members adopt an AB₅ molecular configuration (see above and FIG. 1a. in Johannes & W. Römer, Nature Reviews Microbiology 8, 105-116, 2010). Although STxB forms a doughnut-shaped structure with a central pore into which the carboxyl terminus of STxA inserts, it should be noted that in the absence of STxA, STxB still adopts a pentameric structure that is functionally equivalent to the holotoxin in receptor binding (Johannes & W. Römer, Nature Reviews Microbiology 8, 105-116, 2010).

The inventors proposed to rely on the fact that STxB is a naturally selected binder of glycosphingolipids. So far, no one successfully achieved implementation of the phage display technique with the view to find matching associations of STxB variants (susceptible to act as active compounds), with a range of GSLs.

The experiments reported herein enabled the definition of innovative strategies and means to overcome the barriers summarized above. They rely on experiments specifically carried out, which take into account the particular nature of the STxB protein, and surprisingly proved to be successful.

As a proof of concept, the inventors successfully displayed a so-called STxB protein and an STxB protein mutant on a M13 bacteriophage, while conserving the functional integrity of the STxB moiety. The inventors accordingly defined specifically adapted screening strategies to meet the above needs, and could identify several mutants keeping glycosphingolipids binding properties. Part of the invention, the inventors also refined the model of STxB scaffolds known in the art, and possible variations that can be brought to the same.

### DESCRIPTION OF THE INVENTION

The present invention accordingly provides a polypeptide having a length from 55 to 85 amino-acid residues:
a) whose polypeptidic sequence comprises or consists essentially of or consists of the consensus sequence: XaPDCVTGKVEYTKYNXbXcXdTFXeVKVGDKXfXgXhXiXjXkXlXmLQSLLLSAQITGMT VTIKXnXoXpCHNXqGXrXsXtEVIFR (SEQ ID NO: 2) where
   Xa is selected among: T, A or S, and
   Xb, Xc, Xd, Xf, Xm are independently selected among: D, E or N, and
   Xe, Xi, Xn, Xp, Xt are independently selected among: T, A or S, and
   Xg is selected among: L, I or V, and
   Xh is selected among: F, Y, W or A, and
   Xj, is selected among: N, E or S, and
   Xk is selected among: R, K or E, and
   Xl is selected among: W, F, Y or A, and
   Xo is selected among: N, E, D or S, and
   Xq is selected among: G A or S, and
   Xr is selected among: G, A, S or T and
   Xs is selected among: F, L or Y,
especially provided that when Xa is T or A, Xb, Xc, Xd are not D, Xe is not T, Xf is not E, Xg is not L, Xh is not F, Xi is not T, Xj is not N, Xk is not R, Xl is not W, Xm is not N, Xn is not T, Xo is not N, Xp is not A, Xq is not G, Xr is not G, Xs is not F and Xt is not S,
and/or,
b) whose polypeptidic sequence comprises or consists essentially of or consists of the consensus sequence: XaPDCVTGKVEYTKYNXbDDTFXeVKVGDKEXgXhTXjXkWNLQSLLLSAQITGMTVTIK XnNXpCHNGGXrXsXtEVIFR (SEQ ID NO: 37) where Xa, Xb, Xe, Xg, Xh, Xj, Xk, Xn, Xp, Xr, Xs, Xt are as defined in point a),
and/or,
c) whose polypeptidic sequence comprises or consists essentially of a sequence having for structure Xa(S1)XbXcXd(S2)Xe(S3)XfXgXhXiXjXkXIXm(S4)XnXoXp(S5)Xq(S6)XrXsXt(S7) in which 51, S2, S3, S4, S5, S6 and S7, in this order from the N-terminus to the C-terminus of the polypeptide, are defined as follows:
   S1 represents the amino-acid sequence PDCVTGKVEYTKYN (SEQ ID NO: 38),
   S2 represents the amino-acid sequence TF
   S3 represents the amino-acid sequence VKVGDK (SEQ ID NO: 39),
   S4 represents the amino-acid sequence LQSLLLSAQITGMTVTIK (SEQ ID NO: 40),
   S5 represents the amino-acid sequence CHN
   S6 represents amino-acid residue G, and
   S7 represents the amino-acid sequence EVIFR (SEQ ID NO: 41),
      and wherein Xa, Xb, Xc, Xd, Xe, Xf, Xg, Xh, Xi, Xj, Xk, XI, Xm, Xn, Xo, Xp, Xq, Xr, Xs, Xt are amino-acids as defined in point a) above, in particular a polypeptide whose polypeptidic sequence keeps at least 80% identity with SEQ ID NO: 1, and/or differs from SEQ ID NO: 1 by one or several conservative amino acid substitution(s),
and/or,
d) whose polypeptidic sequence comprises or consists essentially of or consists of a fragment, especially a fragment of contiguous amino-acid residues of at least 55 amino-acid residues, of any one of the sequences defined in a), b) or c), or comprises or consists essentially of or consists of a portion of any one of the sequences defined in a), b) or c) over a length of at least 55 amino-acid residues;
to the proviso that the polypeptide does not consists of SEQ ID NO: 1 or SEQ ID NO: 32 or SEQ ID NO: 36, or SEQ ID NO: 43, or SEQ ID NO: 44, or SEQ ID NO: 45, or SEQ ID NO: 46, or SEQ ID NO: 47, or SEQ ID NO: 48, or SEQ ID NO: 49, or SEQ ID NO: 50, or SEQ ID NO: 51, or SEQ ID NO: 52, or SEQ ID NO: 53.

Table 1 below shows the correspondence of defined consensus sequences with amino-acid positions of SEQ ID NO: 1, and defines the size of the “variable” and “scaffold” regions referred to herein (as sections of the consensus sequences defined by the inventors). It will be understood that the skilled person in the art can readily determine amino acid positions of a polypeptide sequence to check whether they correspond to the positions defined in Table 1, by making a correspondence with the sequence of SEQ ID NO: 1.

TABLE 1

| | | | Corresponding | Linking |
|---|---|---|---|---|
| | | | AA | fragment |
| | | Correspondance | positions of | size/ |
| Section | | with | SEQ ID NO: | Scaffold |
| name | Abv. | SEQ ID NO: 2 | 1 | size |
| Variable | V1 | Xa | 1 | 1 |
| section V1 | | | | |
| Scaffold | S1 | PDCVTGKVEYTKYN | 2-15 | 14 |
| section S1 | | | | |
| Variable | V2 | XbXcXd | 16-18 | 3 |
| section V2 | | | | |
| Scaffold | S2 | TF | 19-20 | 2 |
| section S2 | | | | |
| Variable | V3 | Xe | 21 | 1 |
| section V3 | | | | |
| Scaffold | S3 | VKVGDK | 22-27 | 6 |
| section S3 | | | | |
| Variable | V4 | XfXgXhXiXjXkXl | 28-35 | 8 |
| section V4 | | Xm | | |
| Scaffold | S4 | LQSLLLSAQITGMT | 36-53 | 18 |
| section S4 | | VTIK | | |
| Variable | V5 | XnXoXp | 54-56 | 3 |
| section V5 | | | | |
| Scaffold | S5 | CHN | 57-59 | 3 |
| section S5 | | | | |
| Variable | V6 | Xq | 60 | 1 |
| section V6 | | | | |
| Scaffold | S6 | G | 61 | 1 |
| section S6 | | | | |
| Variable | V7 | XrXsXt | 62-64 | 3 |
| section V7 | | | | |
| Scaffold | S7 | EVIFR | 65-69 | 5 |
| section S7 | | | | |

It will also be understood that the polypeptides of the present invention can be alternatively be defined has having a structure encompassing a sequence (according to the common language definition of this word) of variable and scaffold sections as defined in Table 1.

Accordingly, the sequence Xa(S1)XbXcXd(S2)Xe(S3)XfXgXhXiXjXkXIXm(S4)XnXoXp(S5)Xq(S6)XrXsXt(S7) of point c) above can also be written V1(S1)V2(S2)V3(S3)V4(S4)V5(S5)V6(S6)V7(S7) with Variables and Scaffold sections as defined in Table 1. In fact, the Scaffold sections define portions of polypeptides that remain unchanged with respect to SEQ ID NO: 1, whereas Variable sections constitute linking fragment that can display variability, according to the possible amino acid substitution(s) defined in Table 1.

When fragment polypeptides are considered, as defined in point d) above, it is meant a (fragment) polypeptide whose sequence retains the scaffold sections (defined in Table 1) that can actually be displayed by such a fragment given its length, and as consecutively found from the N-terminus to the C-terminus of said fragment. Even if the fragment polypeptide is shortened at its N-terminus and/or C-terminus extremities, it may retain Scaffold and Variable sections as defined herein, to the exception of sections that cannot be present because of the fragment size.

According to a particular embodiment however, of polypeptide as defined herein can still differ from SEQ ID NO: 1 by one or several conservative amino acid substitution(s), preferably within Variable sections as defined herein, to the extent that the “one or several” substitution(s) remain(s) within the extent defined hereafter.

According to another or cumulative aspect, a polypeptide as defined herein may still keep at least 80% identity with SEQ ID NO: 1, or more, according to the definitions of identity percentages defined in the present description.

In a particular embodiment however, since Scaffold sections are by definition portions of polypeptides that are conceived to remain unmodified, a polypeptide of the invention may keep 100% identity with SEQ ID NO: 1 within said so-called “Scaffold” sections.

Considering consensus sequence XaPDCVTGKVEYTKYNXbDDTFXeVKVGDKEXgXhTXjXkWNLQSLLLSAQITGMTVTIK XnNXpCHNGGXrXsXtEVIFR (SEQ ID NO: 37) defined in point b) above, it will be understood that said sequence corresponds to SEQ ID NO: 2, in which amino acid residues at positions 17, 18, 28, 31, 34, 35, 55, 60 are those found in corresponding positions of SEQ ID NO: 1, which leaves 11 remaining variable positions, i.e., those of positions 1, 16, 21, 29-30, 32-33, 54, 56, and 62-64, according to the variability defined in Table 1 for those positions. Conversely, fixed positions with respect to SEQ ID NO: 1, for the Scaffold defined in SEQ ID NO: 37), are: positions 2-15, 17-20, 22-28, 31, 34-53, 55, 57-61.

According to particular independent embodiments, are encompassed polypeptides having a length of 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or 85 amino acid residues, or a length between any interval that can be defined on the basis of such values. According to particular independent embodiments, are encompassed polypeptides having a length between 55 and 83 amino-acids, or between 65 and 73 amino-acids, especially polypeptides having a length of about 69 amino-acids according to the variability disclosed above and according to any interval of 5 amino-acids encompassing such a length of 69 amino-acids. According to a specific embodiment, a polypeptide of the invention has a length of 69 amino-acids. The same values as disclosed in the present paragraph, but up to 69 amino-acids, or all possible interval(s) of such values, up to 69 amino-acids, apply for appropriate definition of the length of contiguous amino-acid residues defined in point d).

Point c) above concerns “variant polypeptides”, meaning polypeptides resulting from limited variations with respect to its reference sequence, which is SEQ ID NO: 1. Variant polypeptides of the invention, encompass polypeptides having at least 80%, 81%, 82%, 83%, 84%, 85%, 86%, 87%, 88%, 89%, 90%, 91%, 92%, 93%, 94%, 95%, 96%, 97%, 98%, or 99% identity with the sequence of reference, preferably at least 85% or at least 90% or at least 95% or 99% identity with the sequence of reference.

By “identity”, it is meant that the percentage of conserved amino-acid residues when a variant polypeptides is aligned with its reference sequence through conventional alignment algorithms is substantial, meaning that this percentage is at least one of those disclosed above, in particular at least 80%.

Identity percentages can conventionally be calculated through local, preferably global, sequence alignment algorithms and their available computerized implementations. In a most preferred embodiment, identity percentages are calculated over the entire length of the compared sequences. Optimal alignment of amino-acid sequences for comparison can for example be conducted by the local algorithm of Smith & Waterman Adv. Appl. Math. 2: 482 (1981), which is a general local alignment method based on dynamic programming, by the alignment algorithm of Needleman & Wunsch, J. Mol. Biol. 48: 443 (1970), which is also based on dynamic programming, by the search for similarity method of Pearson & Lipman, Proc. Nat'l. Acad. Sci. USA 85: 2444 (1988), or by visual inspection. Computerized implementations of these algorithms are associated with default parameters, which can be used.

A common implementation of a local sequence alignment uses the BLAST analysis, which is described in Altschul et al., J. Mol. Biol. 215: 403-410 (1990). Software for performing BLAST analyses is publicly available. For amino acid sequences, the BLAST program uses as defaults a wordsize (W) of 3, an expectation (E-value cutoff) of 10, and the BLOSUM62 scoring matrix (see Henikoff & Henikoff, Proc. Natl. Acad. Sci. USA 89: 10915 (1989)). Additionally, gap opening may be set at 11, and gap extension at 1. Local alignments are more useful for dissimilar sequences that are suspected to contain regions of similarity or similar sequence motifs within their larger sequence context.

Global alignments, which attempt to align every residue in every sequence, are most useful when the sequences in the query set are similar and of roughly equal size. (This does not mean global alignments cannot start and/or end in gaps.) A general global alignment technique is the Needleman-Wunsch algorithm, which may be used according to default parameters readily accessible to the skilled person.

Another suitable sequence alignment algorithm is, according to a particular embodiment, a string matching algorithm, such as KERR (Dufresne et al., Nature Biotechnology, Vol. 20, December 2002, 1269-1271). KERR computes the minimal number of differences between two sequences, by trying to optimally fit the shorter sequence into the longer one. KERR delivers the percent identity to the whole subject sequence. In this respect, it is preferred that identity percentages are calculated over the entire length of each of the compared sequences.

In addition, or independently of any identity percentage with a sequence of reference as defined herein, polypeptides of the invention also encompass polypeptides having a sequence differing from the sequence of reference defined herein, by one or several conservative amino acid substitution(s). Conservative substitutions encompass a change of residues made in consideration of specific properties of amino acid residues as disclosed in the following groups of amino acid residues and the resulting substituted peptidomimetic should not be modified functionally:

Acidic: Asp, Glu;

Basic: Asn, Gln, His, Lys, Arg;

Aromatic: Trp, Tyr, Phe;

Uncharged Polar Side chains: Asn, Gly, Gln, Cys, Ser, Thr, Tyr;

Nonpolar Side chains: Ala, Val, Leu, Ile, Pro, Phe, Met, Trp;

Hydrophobic: Ile, Val, Leu, Phe, Cys, Met, Nor;

Neutral Hydrophilic: Cys, Ser, Thr;

Residues impacting chain orientation: Gly, Pro

Small amino acid residues: Gly, Ala, Ser.

By “one or several”, it is meant any number consistent with the length of the polypeptide, and optionally consistent with the identity percentages defined above. According to a particular embodiment, by “several”, it is meant 1, 2, 3, 4, 5, 6, 7, 8, 9, or 10.

As detailed above, according to a particular aspect, a polypeptide as defined herein may differ from SEQ ID NO: 1 by one or several conservative amino acid substitution(s), preferably within variable sections as defined herein, i.e., it differs, to the extent that the substitution is “conservative” from the choices allowed for amino-acids Xa to Xt defined herein, at any one or several positions that can vary according to the present disclosure.

According to a particular embodiment, a polypeptide of the invention comprises or consists essentially of or consists of a fragment, especially a fragment of contiguous amino-acid residues of at least 55 amino-acid residues, of any one of the sequences defined in a), b) or c) or herein, or comprises or consists essentially of or consists of a portion of any one of the sequences defined in a), b) or c) over a length of at least 55 amino-acid residues.

From the above, it will be understood that, according to particular embodiments, a polypeptide described herein can nevertheless further vary with respect to a consensus sequence as defined herein, which sets scaffold regions that are determined through said consensus sequence, by point conservative substitutions/mutations as defined herein. Such point conservative substitutions/mutations can affect any one of the scaffold amino-acid residues as defined herein, alone or in all combinations with the possibilities of variation offered in the variable regions. According to a particular embodiment, point conservative substitutions/mutations as defined herein can affect any one or several amino-acid residues selected among: amino-acid residue(s) found at position(s) 2, 3, 4, 5, 6, 7, 8, 9, 10, 11, 12, 13, 14, 15, 17, 18, 19, 20, 22, 23, 24, 25, 26, 27, 28, 30, 31, 34, 35, 36, 37, 38, 39, 40, 41, 42, 43, 44, 45, 46, 47, 48, 49, 50, 51, 52, 53, 55, 57, 58, 59, 60, 61, preferably 1, 2 or 3 of those positions only, to the proviso that the resulting sequence is not disclaimed herein, or outside the other requirements defined herein.

Also, such variations may be authorized to the proviso that the polypeptide retains functional properties as defined herein, of the non-mutated embodiment. The skilled person can readily compare such properties, using the experiments and guidance provided in the present description.

According to a particular embodiment, a polypeptide of the invention has the capability, when found under a pentameric form, to bind with, especially to specifically bind to, a single or several glycosphingolipid(s) selected from the group consisting of: Gb3, Gb4, Forsmann like iGb4, fucosyl-GM1, GM1, GM2, GD2, Globo-H, NeuAc-GM3, NeuGc-GM3, GD1a, O-acetyl-GD3, O-acteyl-GD2, O-acetyl-GT3, GD3, and mixtures thereof.

These glycosphingolipids are commonly known and described in the art: correspondence with their known synonyms and/or systematic names are provided in Table 2. They are in particular identified by reference to database entries (LM ID), provided by reference to the Lipid Maps® Structure Database available at http://www.lipidmaps.org/data/structure/LMSDSearch.php?Mode=SetupTextOntologySearch (Sud et al. (2007). LMSD: LIPID MAPS structure database. Nucleic Acids Research, 35(SUPPL. 1), 527-532. https://doi.org/10.1093/nar/gk1838). Corresponding cancer types in which these GSLs can be found expressed are also provided. It will be appreciated that one of the universal changes in cancer is glycosylation at the surface of tumoral cells, and carbohydrate-binding proteins can be produced to selectively recognize tumor cells over normal tissues. A common feature is the over-expression of GSL at the surface of the tumor. GSLs are a known and promising group of cell surface targets.

TABLE 2

| GSL common | | LM ID of | |
|---|---|---|---|
| name (s) (with | | LIPID | |
| alternative common | | MAPS | |
| names or synonyms) | Systematic name | database | Cancer types |
| Gb3, | Galα1-4Galβ1-4Glcβ- | LMSP0502AA00 | ovarian cancer, |
| Globotriaosylceramide, | Cer | | breast |
| CD77, ceramide | | | carcinoma, |
| trihexoside | | | Colon cancer, |
| | | | Gastric |
| | | | adenocarcinoma, |
| | | | Burkitt's |
| | | | lymphoma |
| Gb4, Globoside, | GalNAcβ1-3Galα1- | LMSP0502AB00 | colon carcinoma, |
| Gb4Cer, P antigen | 4Galβ1-4Glcβ-Cer | | breast |
| | | | carcinoma |
| Forsmann like iGb4 | GalNAcα1-3GalNAcβ1- | LMSP0502AC00 | melanoma |
| | 3Galα1-3Galβ1-4Glcβ- | | |
| | Cer | | |
| fucosyl-GM1, | Fucα1-2Galβ1- | LMSP0601BD00 | small cell lung |
| Fuc-GM1 | 3GalNAcβ1-4(NeuAcα2- | | cancer (SCLC) |
| | 3)Galβ1-4Glcβ-Cer | | |
| GM1 | Galβ1-3GalNAcβ1- | LMSP0601AP00 | renal carcinoma, |
| | 4(NeuAcα2-3)Galβ1- | | Neuroblastoma |
| | 4Glcβ-Cer | | |
| GM2 | GalNAcβ1-4(NeuAcα2- | LMSP0601AM00 | Melanoma, |
| | 3)Galβ1-4Glcβ-Cer | | Cervical |
| | | | carcinoma, |
| | | | Neuroblastoma, |
| | | | Glioblastoma, |
| | | | SCLC, renal |
| | | | carcinoma |
| GD2 | GalNAcβ1-4(NeuAcα2- | LMSP0601AN00 | Melanoma, |
| | 8NeuAcα2-3)Galβ1- | | Neuroblastoma, |
| | 4Glcβ-Cer | | Glioma, SCLC |
| Globo-H, type IV H | Fucα1-2Galβ1- | LMSP0502AI00 | Cancers of |
| | 3GalNAcβ1-3Galα1- | | epithelial origins |
| | 4Galβ1-4Glcβ-Cer | | such as non- |
| | | | small cell lung |
| | | | cancer and |
| | | | breast cancer, or |
| | | | cancers selected |
| | | | among: breast, |
| | | | colon, |
| | | | endometrial, |
| | | | gastric, |
| | | | pancreatic, lung, |
| | | | and prostate |
| | | | cancers |
| NeuAc-GM3 | NeuAcα2-3Galβ1- | LMSP0601AJ00 | Melanoma, |
| | 4Glcβ-Cer | | breast |
| | | | carcinoma, renal |
| | | | carcinoma |
| NeuGc-GM3 | NeuGcα2-3Galβ1- | LMSP0601AF00 | Retinoblastoma, |
| | 4Glcβ-Cer | | Colon cancer, |
| | | | Melanoma, |
| | | | Breast |
| | | | carcinoma, |
| | | | Neuroectodermal |
| | | | cancer, non- |
| | | | small cell |
| | | | lung cancer |
| | | | (NSCLC), Wilms |
| | | | tumor |
| GD1a | NeuAcα2-3Galβ1- | LMSP0601AS00 | ovarian cancer |
| | 3GalNAcβ1-4(NeuAcα2- | | |
| | 3)Galβ1-4Glcβ-Cer | | |
| O-acetyl-GD3 | 9-OAc-NeuAca2- | LMSP0601BK00 | breast |
| | 8NeuAcα2-8NeuAcα2- | | carcinoma |
| | 3Galβ1-4Glcβ-Cer | | |
| O-acteyl-GD2 | GalNAcβ1-4(9- | LMSP0601DA00 | Neuroblastoma, |
| | OAcNeuAcα2- | | Glioblastoma |
| | 8NeuAcα2-8NeuAcα2- | | |
| | 3)Galβ1-4Glcβ-Cer | | |
| O-acetyl-GT3 | 9-OAc-NeuAcα2- | LMSP0601BK00 | Breast |
| | 8NeuAcα2-8NeuAcα2- | | carcinoma |
| | 3Galβ1-4Glcβ-Cer | | |
| GD3 | NeuAcα2-8NeuAcα2- | LMSP0601AK00 | Melanoma, |
| | 3Galβ1-4Glcβ-Cer | | neuroblastoma, |
| | | | Osteosarcoma, |
| | | | Glioma, t-All |

It is observed that STxB is a protein that naturally pentamerizes when its constitutive monomers are found in solution. According to a particular embodiment, the pentameric form referred to herein is an homopentameric form, where all subunits are the same.

Shiga toxin has been shown to bind to whole cells with a binding constant of 10⁹ M⁻¹ (‘Pathogenesis of Shigella Diarrhea: Rabbit Intestinal Cell Microvillus Membrane Binding Site for Shigella Toxin’; Fuchs, G., Mobassaleh, M., Donohue-Rolfe, A., Montgomery, R. K., Gerard, R. J., and Keusch, G. T. (1986) Infect. Immun. 53, 372-377); however, the binding constant for soluble Gb3 is only about 10³ M⁻¹ (‘Interaction of the Shiga-like Toxin Type 1 B-Subunit with Its Carbohydrate Receptor’ St. Hilaire, P. M., Boyd, M. K., and Toone, E. J. (1994) Biochemistry 33, 14452-14463).

According to a particular embodiment, polypeptides of the invention, when found under a pentameric form associating five polypeptides as defined herein, especially five identical polypeptides as defined herein, bind one, or at least one of their targets as defined herein with an affinity greater than 10² M⁻¹ (measured by ITC (isothermal titration calorimetry) with soluble carbohydrate corresponding to the target GSL), and/or an apparent affinity for membrane containing the corresponding GSL target (Cells/liposomes) greater than 10⁶ M⁻¹ (measured by SPR (surface plasmon resonance)).

The invention therefore relates to a polypeptide, which has one or several of the following property(ies) when found under a pentameric form associating five polypeptides as defined herein, especially five identical polypeptides as defined herein:
a. the property to bind to a glycosphingolipid selected from the group consisting of: Gb3, Gb4, Forsmann like iGb4, fucosyl-GM1, GM1, GM2, GD2, Globo-H, NeuAc-GM3, NeuGc-GM3, GD1a, O-acetyl-GD3, O-acteyl-GD2, O-acetyl-GT3, GD3, and mixtures thereof and/or
b. an affinity for its target greater than 10² M⁻¹ (measured by ITC), in particular greater than or about 10³ M⁻¹ and/or
c. an apparent affinity for a membrane displaying its target greater than 10⁶ M⁻¹ (measured by SPR), in particular greater than 10⁷⁰ or greater than 10⁸, or greater or about 10⁹ M⁻¹.

By “affinity”, reference is made to the physical strength of the interaction between pentamerized polypeptides of the invention and its target. Affinity of the pentamerized polypeptides of the invention for its target as defined herein may be measured by a Kd value that is equal or less than 100 μM, or equal or less than 90, 80, 70, 60, 50, 40, 30, 20, 10, 5, 4, 3, 2 or 1 μM, more particularly in the ranges of: 0.5 to 10 μM, 1 to 10 μM, 1 to 5 μM or 0.5, measured by SPR (Surface Plasmon Resonance) analysis. Affinity can be defined by a Kd_{eq} value (also designated Kd) and can be measured by methods conventional in the art, in particular methods reported herein, especially by SPR analysis. Reference is also made to Gallegos et al., “Shiga Toxin Binding to Glycolipids and Glycans”. PLoS ONE 7(2): e30368, for relevant explanations with respect to target affinity measurement using ITC, illustrative of the method and ranges known to the skilled person in the art. Reference is more particularly made to the first section of the “Results” at page 2 (“Characterization of individual glycan binding sites by ITC”), and the “Isothermal Titration calorimetry” paragraph in the “Material and Methods” section at page 9, which are incorporated by reference.

Further means for assessing the functionality of polypeptides of the invention, as commonly known are readily practicable by the skilled person following his/her knowledge or indications widely available in the literature include:
Binding on cells (FACS, immunofluorescence microscopy), and/or
Internalization experiment (on purified polypeptides): polypeptides are put into contact with cells at 4° C., and then incubated at 37° C. for at least 45 minutes to assess internalization and trafficking into the cells (for STxB WT, STxB must arrive in the Golgi apparatus)

According to a particular embodiment, a polypeptide of the invention comprises or consists essentially of or consists of any one of the following sequences: SEQ ID NO:3, SEQ ID NO:5, SEQ ID NO:7, SEQ ID NO:9, and SEQ ID NO:11.

These sequences correspond respectively to the hits and clones found by the inventors as binding specifically to Gb3 (see Experimental Section herein), corresponding, respectively, to:
clones A3-D10-H3 (3 replicates in the final pool after selection),
clones B12-C03-D12-G05-G11-H11 (6 replicates in the final pool after selection)
clones A06-C06 (2 replicates in the final pool after selection)
clone B02 (unique sequence after selection)
clone B05 (unique sequence after selection).

These clones are all “variant polypeptides” according to the definitions provided herein, which have between 83 and 93% identity with SEQ ID NO: 1, as defined through a conventional BLAST algorithm, but retain the Scaffold sections defined by the inventors. According to particular embodiments, are also encompassed within the present invention continuous fragments of these variants, as defined in point c) above.

It will be understood that these sequences fall within the definition of the consensus sequence defined herein as (SEQ ID NO: 37): XaPDCVTGKVEYTKYNXbDDTFXeVKVGDKEXgXhTXjXkWNLQSLLLSAQITGMTVTIK XnNXpCHNGGXrXsXtEVIFR where Xa, Xb, Xe, Xg, Xh, Xj, Xk, Xn, Xp, Xr, Xs, Xt are as defined above. This consensus sequence leaves 11 remaining variable positions, i.e., those of positions 1, 16, 21, 29-30, 32-33, 54, 56, and 62-64, according to the variability defined in Table 1 for those positions. Conversely, fixed positions with respect to SEQ ID NO: 1, for the Scaffold defined in SEQ ID NO: 37), are: positions 2-15, 17-20, 22-28, 31, 34-53, 55, 57-61.

Nucleic acid sequences encoding respectively SEQ ID NO:3, SEQ ID NO:5, SEQ ID NO:7, SEQ ID NO:9, and SEQ ID NO:11 are provided, respectively, under SEQ ID NO:4, SEQ ID NO:6, SEQ ID NO:8, SEQ ID NO:10, and SEQ ID NO:12.

In particular, polypeptides of the invention may specifically bind a glycosphingolipid (GSL), especially as defined herein (in particular they bind one of the species listed in Table 2, or several of them, according to the definition of “several” defined above), expressed by cells/tissue and/or to detect particular glycosphingolipids in a sample, when those polypeptides are found under a pentameric form as defined herein.

According to a particular embodiment, polypeptides of the invention found under a pentameric form as defined herein, specifically bind Gb3.

According to a more specific embodiment, polypeptides of the invention defined according to or with respect to SEQ ID NO: 37, including variants or fragments thereof, have the property to bind Gb3 when found under a pentameric form as defined herein.

The inventors have specifically designed starting from the B-subunit of Shiga toxin (STxB) sequence TPDCVTGKVEYTKYNDDDTFTVKVGDKELFTNRWNLQSLLLSAQITGMTVTIKTNACH NGGGFSEVIFR (SEQ ID NO: 1), new peptides comprising particular mutation sites, as described herein. These new peptides may advantageously be able to bind to other glycosphingolipids than Gb3, especially which are known, for example, to be newly expressed or overexpressed on tumor cells.

It is defined that the terms peptide(s) and polypeptide(s) is/are used interchangeably herein.

In particular, the peptides of the present invention can form pentameric structures similar to the STxB's own pentameric structure, even when bound to a host, and can therefore specifically bind and target glycosphingolipids.

This technical effect is very important since the conventional means for targeting disease relevant antigens, antibodies, do not perform well with glycosphingolipids, due to their poor immunogenicity. To the contrary the peptides of the invention provide efficient means for detecting/targeting glycosphingolipids.

The invention also relates to a pentameric assembly, especially an homopentameric assembly, of polypeptides as defined herein (i.e., an homopentameric assembly of identical polypeptides), since STxB monomers spontaneously pentamerize in solution, and show functional properties under this form.

According to particular embodiments, the pentameric especially homopentametic assembly of polypeptides of the invention encompass at least one polypeptide that is further modified in its sequence with respect to the sequence of a “natural” monomer as found in the nature, so as to enable coupling and/or conjugation of the resulting assembly of polypeptides with active ingredients, or enable the resulting assembly of polypeptides to be labelled.

It is known in the art that functional STxB can be coupled and/or conjugated with active ingredients. Non-limitative examples encompass cytotoxic molecules (such as maytansinoids, auristatin, calicheamicin, duocarmicin, and daunorubicin), or contrast agents, or antigens. Functional STxB can also be found associated with A-subunit of Shiga toxin.

Accordingly, the sequence of at least one single polypeptide monomer, alone or as found within an assembly as described herein, can be conventionally modified with respect to a so-called STxB sequence as naturally found in the nature, through N-terminal, internal or C-terminal peptidic modifications, and/or be coupled to an active ingredient, and/or be labelled.

N-terminal modifications can (non-limitatively) encompass: acetylation, biotinylation, dansyl labelled extremity, 2,4-dinitrophenyl (2,4-DNP) attached to the N-terminal extremity (or internally through a lysine side chain), fluorescein labelling, 7-methoxycoumarin acetic acid (Mca) labelling, palmitic acid conjugation, addition of a further amino acid residue at the extremity, such as a Cysteine residue for coupling with labels or drugs, or a residue adapted to this end.

Internal modifications can (non-limitatively) encompass: presence of isotope labelled amino-acids, phosphorylation of amino acids (especially Tyr, Ser and Thr residues), addition of a spacer (in particular for a cargo that is a drug, a dye, a tag, therefore avoiding or reducing steric hindrance with respect to the binding sites of the pentameric assembly): PEGylation, amino hexanoic acid spacer.

C-terminal modifications can (non-limitatively) encompass amidation, or addition of a further amino acid residue at the extremity, such as a Cysteine residue for coupling with labels or drugs, or a residue adapted to this end. Modifications can also encompass those appropriate for the use of bioorthogonal chemistry, especially click bioorthogonal chemistry. For instance, bioorthogonal functional groups such as azide, cyclooctyne or alcine can be introduced in order to make use of such chemical ligation strategies.

In the present invention, the peptides disclosed herein may be fused with any compound or protein known to one skilled in the art.

It is observed that when polypeptides of the invention are expressed by cells, they may be found with a peptide signal fused at their N-terminal extremity. Such a peptide signal may have the sequence MKKTLLIAASLSFFSASALA (SEQ ID NO: 13).

For instance, the polypeptide of SEQ ID NO: 1 fused to SEQ ID NO: 13 is identified under SEQ ID NO: 14, and is part of the present disclosure.

A nucleic acid sequence encoding SEQ ID NO: 13 is provided under SEQ ID NO: 15. SEQ ID NO: 15 may accordingly be found fused to any nucleic acid molecule, part of the invention, which encodes a polypeptide of the invention as defined herein.

Another object of the present invention is also to provide a fusion protein comprising a peptide of the invention or a peptide of sequence TPDCVTGKVEYTKYNDDDTFTVKVGDKELFTNRWNLQSLLLSAQITGMTVTIKTNACH NGGGFSEVIFR (SEQ ID NO: 1) fused to a coat protein of a virus, such as phage.

In the present disclosure, the term “coat protein” means a protein, at least a portion of which is present on the surface of the virus particle. From a functional perspective, a coat protein is any protein which associates with a virus particle during the viral assembly process in a host cell, and remains associated with the assembled virus until it infects another host cell. The coat protein may be a major coat protein or a minor coat protein. A “major” coat protein is a coat protein which is present in the viral coat at 10 copies of the protein or more.

According to the disclosure, the coat protein may be selected from the group comprising pIII protein and pVIII protein phage coat protein. Advantageously, the coat protein may be pIII protein phage coat protein, for example from M13 bacteriophage.

In the present invention, the term “fusion protein” means a polypeptide having two portions covalently linked together, where each of the portions is a polypeptide having a different property. The property may be a biological property, such as activity in vitro or in vivo. The property may also be a simple chemical or physical property, such as the ability to bind a target molecule, or the ability to catalyze a reaction, and so on. The two portions may be linked directly by a single peptide bond or through a peptide linker containing one or more amino acid residues. Generally, the two portions and the linker will be in the reading frame, for example the same open reading frame, with each other.

According to a particular embodiment, a fusion protein comprises a polypeptide as defined in any one of the embodiments disclosed herein or a polypeptide consisting of SEQ ID NO: 1, which is fused to a coat protein of a virus or a portion of a coat protein of a virus.

According to a particular embodiment, a fusion protein comprises a polypeptide whose polypeptidic sequence comprises or consists essentially of or consists in a sequence having at least 80% identity with SEQ ID NO: 1, and/or differing from SEQ ID NO: 1 by one or several conservative amino acid substitution(s), according to the definitions provided above.

According to a particular embodiment the coat protein is a pIII phage coat protein, in particular a pIII phage coat protein of a M13 bacteriophage, especially the pIII phage coat protein as defined in SEQ ID NO: 16.

For example, such a fusion protein may have the sequence of SEQ ID NO: 17, with, from positions 3 to 71 a STxB sequence (as for instance defined under SEQ ID NO: 1), from positions 72 to 74 a small linker, from positions 75 to 80 a 6H histidine tag, from positions 81 to 122 a Myc tag (three repeats), from positions 123 to 126 a small linker GAA, then a Q residue left in bacteria such as TG1 bacteria (amber-suppressor Host), and a pIII fragment as defined herein (SEQ ID NO: 16). In SEQ ID NO: 17, amino-acid residues of positions 1 and 2 come with the restriction sites (NcoI) for cloning. The reading frame thus starts with MA, but the encoded STxB protein really starts at position 3.

A nucleic acid sequence encoding SEQ ID NO: 17 is provided under SEQ ID NO: 18. Illustrating a particular embodiment, SEQ ID NO: 18 includes amber stop codon TAG from positions 376 to 378.

A nucleic acid sequence encoding SEQ ID NO: 16 is provided under SEQ ID NO: 19.

According to a particular embodiment, a fusion protein of the invention consists of any one of the following sequences: SEQ ID NO:20, SEQ ID NO:22, SEQ ID NO:24, SEQ ID NO:26, and SEQ ID NO:28, encompassing respectively SEQ ID NO: 3, SEQ ID NO:5, SEQ ID NO:7, SEQ ID NO:9, and SEQ ID NO:11 as defined herein, as a STxB variant polypeptide.

According to a particular embodiment, a fusion protein of the invention consists of SEQ ID NO: 33, encompassing SEQ ID NO: 32 as defined hereafter, as a STxB variant polypeptide.

Nucleic acid sequences encoding respectively SEQ ID NO:20, SEQ ID NO:22, SEQ ID NO:24, SEQ ID NO:26, and SEQ ID NO:28 are provided, respectively, under SEQ ID NO:21, SEQ ID NO:23, SEQ ID NO:25, SEQ ID NO:27, and SEQ ID NO:29. Illustrating particular embodiments, these sequences include stop codons from positions 376 to 378.

Nucleic acid sequence encoding SEQ ID NO:33 is provided under SEQ ID NO:34.

Another object of the present invention is a nucleic acid molecule encoding a polypeptide as defined herein, according to all encompassed embodiments, and/or a fusion protein of the invention as defined herein, according to all encompassed embodiments.

According to particular embodiments applicable to all nucleic acid molecules defined in the present disclosure, a nucleic acid molecule of the invention has nucleotide sequence, which comprises a stop codon at the end of the sequence encoding the polypeptide of the invention as defined herein or the polypeptide consisting of SEQ ID NO: 1. According to particular aspects, such stop codons are as defined hereafter.

For example, the nucleic acid of the invention may encode for a peptide of amino acid sequence

XaPDCVTGKVEYTKYNXbXcXdTFXeVKVGDKXfXgXhXiXjXkXlXmLQSLLLSA QITGMTVTIKXnXoXpCHNXqGXrXsXtEVIFR (SEQ ID NO: 2)

wherein
Xa is T, A or S,
Xb, Xc, Xd, Xf, Xm are independently D, E or N,
Xe, Xi, Xn, Xp, Xt are independently T, A or S,
Xg is L, I or V,
Xh is F, Y, W or A,
Xj, is N, E or S,
Xk is R, K or E
Xl is W, F, Y or A,
Xo is N, E, D or S,
Xq is G A or S,
Xr is G, A, S or T
Xs is F, L or Y,

fused to a coat protein of a virus, especially according to the definitions provided herein.

According to a particular aspect, a nucleic acid of the invention may also comprise at the end of the sequence coding for peptide of the invention or for the peptide of SEQ ID NO: 1 a stop codon. In other words, the nucleic acid coding for the fusion protein may comprise between the two coding sequences a stop codon. The stop codon may be any stop codon known to one skilled in the art adapted to the present invention. In particular, the stop codon may be an amber stop codon (TAG/UAG).

However, the present invention also encompasses nucleic acid molecules, which do not comprise such stop codons.

In the present invention, the nucleic acid of the invention coding for peptide of the invention, for the peptide of SEQ ID NO: 1 or a fusion protein of the invention may further comprises at its end an extra codon, (UGC/TGC or UGU/TGT) coding for a cysteine.

The nucleic acid of the invention may be any suitable nucleic acid coding sequence encoding peptides or the fusion proteins of the present invention, fragment or derivative thereof. This sequence is preferably useful for manufacturing the peptide or fusion protein of the present invention or a fragment or derivative thereof, for example by transfection.

According to a particular embodiment, and as a further definition of the nucleic acid molecules of the present disclosure, a nucleic acid molecule of the invention is a fusion gene encompassing, from its 3′ to its 5′ extremities:

a. a first nucleic acid sequence encoding polypeptide of the invention as defined herein or the polypeptide consisting of SEQ ID NO: 1, and

b. a second nucleic acid sequence encoding at least a portion of a pIII filamentous phage coat protein, according in particular to the definitions provided herein,

wherein said fusion gene comprises between the first and second nucleic sequences at least one stop codon, according to the definitions provided herein.

According to a particular embodiment, a first nucleic acid sequence is defined as follows: it comprises or consist essentially of or consists of any one of the SEQ ID NO: 30, SEQ ID NO: 31, SEQ ID NO: 4, SEQ ID NO: 6, SEQ ID NO: 8, SEQ ID NO: 10, SEQ ID NO: 12, or comprises or consist essentially of or consists of a nucleic acid sequence having at least 70%, or at least 80%, preferably 85%, more preferably 90% or 95% identity with any one of the SEQ ID NO: 30, SEQ ID NO: 31, SEQ ID NO: 4, SEQ ID NO: 6, SEQ ID NO: 8, SEQ ID NO: 10, SEQ ID NO: 12.

It will be understood that such nucleic acid molecules may comprise nucleic acid sequences encoding a peptide signal, according to the definitions provided herein, at their N-terminal extremities.

It is observed that SEQ ID NO: 31 encodes STxB variant SEQ ID NO: 32, which correspond to STxB variants bearing the two mutations D18E, G62T with respect to SEQ ID NO: 1, and which has been used in the experimental section herein.

According to a particular embodiment, the second nucleic acid sequence is defined as follows: it comprises or consist essentially of or consists of SEQ ID NO: 19, or portion of it, especially over a length of 300 bp.

According to particular embodiment, the stop codon is selected among DNA stop codons: TAG, TAA and TGA.

According to a particular embodiment, a nucleic acid molecule of the invention consists of any one of: SEQ ID NO:21, SEQ ID NO:23, SEQ ID NO:25, SEQ ID NO:27, SEQ ID NO:29, and SEQ ID NO:34.

According to another aspect, a nucleic acid molecule encompassing a nucleic acid molecule as defined herein is also encompassed within the present invention: such a nucleic acid molecule can be a vector, especially a plasmid, more particularly a phagemid or phage vector, or is contained in a vector, especially a plasmid, more particularly a phagemid or phage vector or is a phage genome.

According to a particular embodiment, such a vector is a pHEN2 phagemid comprising a nucleic acid molecule comprising or consisting essentially of or consisting of:
(1) at least one first nucleic acid sequence or a variant thereof as defined herein,
(2) at least one stop codon selected among TAG, TAA and TGA, and
(3) a second nucleic acid sequence as defined herein, in the order of (1), (2) and (3), or a variant thereof.

An example of vector encompassed within the present invention, as used in instant experimental section, is provided under SEQ ID NO: 35.

Another object of the invention is an expression system comprising a plasmid, phagemid and/or an expression vector comprising a nucleic acid coding for the peptide of the invention, a peptide of sequence TPDCVTGKVEYTKYNDDDTFTVKVGDKELFTNRWNLQSLLLSAQITGMTVTIKTNACH NGGGFSEVIFR (SEQ ID NO: 1) or a fusion protein of the invention.

The peptide and fusion protein of the invention as are defined above.

Nucleic acids sequence coding for the peptide of the invention, a peptide of sequence TPDCVTGKVEYTKYNDDDTFTVKVGDKELFTNRWNLQSLLLSAQITGMTVTIKTNACH NGGGFSEVIFR (SEQ ID NO: 1) or a fusion protein of the invention are as defined above.

According to a particular embodiment, an expression system of the invention comprises:

a) a nucleic acid encoding a polypeptide having a length from 55 to 85 amino-acid residues, whose polypeptidic sequence comprises or consists essentially of or consists of the consensus sequence XaPDCVTGKVEYTKYNXbXcXdTFXeVKVGDKXfXgXhXiXjXkXIXmLQSLLLSAQITGMT VTIKXnXoXpCHNXqGXrXsXtEVIFR (SEQ ID NO: 2) where
Xa is selected among: T, A or S, and
Xb, Xc, Xd, Xf, Xm are independently selected among: D, E or N, and
Xe, Xi, Xn, Xp, Xt are independently selected among: T, A or S, and
Xg is selected among: L, I or V, and
Xh is selected among: F, Y, W or A, and
Xj, is selected among: N, E or S, and
Xk is selected among: R, K or E, and
Xl is selected among: W, F, Y or A, and
Xo is selected among: N, E, D or S, and
Xq is selected among: G A or S, and
Xr is selected among: G, A, S or T and
Xs is selected among: F, L or Y,

or,

b) a nucleic acid encoding a polypeptide having a length from 55 to 85 amino-acid residues, whose polypeptidic sequence comprises or consists essentially of or consists in a sequence having at least 75% identity with sequence TDCVTGKVEYTKYNDDDTFTVKVGDKELFTNRWNLQSLLLSAQITGMTVTIKTNACHN GGGFSEVIFR (SEQ ID NO: 1), and/or differing from SEQ ID NO: 1 by one or several conservative amino acid substitution(s),

or,

c) a nucleic acid encoding a polypeptide having a length from 55 to 85 amino-acid residues, whose polypeptidic sequence comprises or consists essentially of or consists of a fragment, especially a fragment of contiguous amino-acid residues of at least 55 amino-acid residues, of any one of the sequences defined in a) or b), or comprises or consists essentially of or consists of a portion of any one of the sequences defined in a) or b) over a length of 55 amino-acid residues,

wherein the expression system is at least one of a plasmid, a phagemid or an expression vector.

It will be understood that lengths and identity percentages are consistently aligned to the values disclosed herein with respect to polypeptides of the invention.

Another object of the invention is an expression system comprising a plasmid, phagemid and/or an expression vector comprising a nucleic acid coding for the fusion protein of the invention, according to any embodiment of the present description.

In the present, the plasmid may be any plasmid known to one skilled in the art adapted for the production of a peptide in a host. It may be for example a plasmid selected from the group comprising pIRES, pIRES2, pcDNA3, pGEX.

In the present, the phagemid may be any phagemid known to one skilled in the art adapted for the production of a peptide in a host. For example, a “phagemid” is a plasmid vector having a bacterial origin of replication, e.g., ColE1, and a copy of an intergenic region of a bacteriophage. The phagemid may be based on any known bacteriophage from one skilled in the art, including filamentous bacteriophage and lambdoid bacteriophage. The plasmid will also preferably contain a selectable marker for antibiotic resistance. Segments of DNA cloned into these vectors can be propagated as plasmids. When cells harbouring these vectors are provided with all genes necessary for the production of phage particles, the mode of replication of the plasmid changes to rolling circle replication to generate copies of one strand of the plasmid DNA and package phage particles. The phagemid may form infectious or non-infectious phage particles. This term includes phagemids which contain a phage coat protein gene or fragment thereof linked to a heterologous polypeptide gene as a gene fusion, such that the heterologous polypeptide is displayed on the surface of the phage particle. (Sambrook et al., Molecular Cloning: A Laboratory Manual, 2nd edition, (1989) Cold Spring Harbor Laboratory Press, Cold Spring Harbor, N.Y., 4.17.) Advantageously, the phagemid is pHEN2 phagemid.

In the present, the expression vector may be any expression vector known to one skilled in the art adapted for the production of a peptide in a host.

In the present the expression system comprises a nucleic acid of the invention in a form suitable for its expression in a host, which means that the plasmid, phagemid, and/or an expression vector include one or more regulatory sequences, selected on the basis of the host to be used for expression, that is operatively linked to the nucleic acid sequence to be expressed. Within a plasmid, phagemid, and/or an expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers, and other expression control elements, for example polyadenylation signals, stop codon, and so on. Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host, and those that direct expression of the nucleotide sequence only in certain hosts, for example tissue-specific regulatory sequences. It will be appreciated by those skilled in the art that the design of the expression system can depend on such factors as the choice of the host to be transformed, the level of expression of protein desired, etc. The expression vectors of the invention can be introduced into host cells to thereby produce proteins or peptides, including fusion proteins or peptides, encoded by nucleic acids as described herein, for example chimeric polypeptides, mutant forms of the chimeric polypeptide, fusion proteins, and so on.

In the present invention, a plasmid, phagemid and/or an expression vector comprising a nucleic acid coding for the peptide of the invention or of sequence (SEQ ID NO: 1) may comprise any promoter adapted known to one skilled in the art. For example, the phagemid may comprise a promoter selected from the group comprising lac promoter, for example LacP, LacO.

In the present invention, the plasmid, phagemid and/or an expression vector comprising a nucleic acid coding for the peptide of the invention or of sequence (SEQ ID NO: 1) may comprise a stop codon at the end of its sequence.

In the present invention, the expression system may comprises a nucleic acid coding for a fusion protein of the invention in which the end of the sequence coding for the peptide of the invention or for sequence SEQ ID NO: 1 may comprise a stop codon. In others words, the nucleic acid coding for the fusion protein may comprise between the two coding sequences a stop codon. Advantageously, the stop codon may be an amber stop codon (UAG).

In the present invention, the expression system may comprise a nucleic acid of the invention coding for peptide of the invention, for the peptide of SEQ ID NO: 1, or a fusion protein of the invention may further comprises at its end an extra codon, (UGC/TGC or UGU/TGT) coding for a cysteine.

In the present invention the expression system may be advantageously a pHEN2 phagemid comprising LacP, LacO promoter in frame with a nucleic acid of the invention coding for a fused protein comprising at the end of the sequence coding for peptide of the invention, or for the peptide of SEQ ID NO: 1, an amber stop codon (UAG), and a sequence coding for a pIII coat protein.

Advantageously, the inventors have demonstrated that, when the expression system is a phagemid, the presence lac promoter, for example LacP, LacO and the presence of an amber stop codon between the nucleic acid coding for the two portions of the fusion protein, i.e. peptide of the invention or of sequence (SEQ ID NO: 1) and the nucleic acid coding for a coat protein of a virus, allows for the expression of either “free” peptide of the invention or of SEQ ID NO: 1, or peptide of the invention, or of SEQ ID NO: 1 fused with said coat protein.

In particular, the inventors have demonstrated that the presence of an amber stop codon between the nucleic acid coding for the two portions of the fusion protein, i.e. peptide of the invention or of sequence (SEQ ID NO: 1) and the one of another protein allows to produce free peptide of the invention or of SEQ ID NO: 1, i.e. not fused, and peptide of the invention or of SEQ ID NO: 1 fused with a coat protein of a virus. The proportion of the “free peptide” compared with the “fused protein” is approximatively or equal to 50% one to one.

In the present, when the expression system comprises a nucleic acid sequence coding for a peptide of the invention or a fusion protein of the invention it may be also introduced into said expression system in frame before and/or after a coding sequence. For example, it may be included into the expression system before and/or after a coding sequence that could allow to improve the recovery of the peptide or fused protein, for example a sequence coding for a tag, for example an histidine tag, a sequence coding for a label protein, for example the Green Fluorescent protein.

One skilled in the art taking into consideration his technical knowledge knows proteins or peptides that could improve the recovery of a molecule, for example a peptide or fused protein.

According to the invention, the peptide of the invention or fusion protein of the invention may represent a peptide library, or a fusion protein library.

According to the invention, the expression systems of the invention comprising nucleic acids of the invention may form an expression system library encoding different peptides of the invention or fusion proteins of the invention.

Another object of the invention is a host comprising a peptide according to the present invention, a fusion protein according to the present invention or a peptide of sequence SEQ ID NO: 1 and/or a nucleic acid sequence according to the present invention or coding for sequence (SEQ ID NO: 1) and/or an expression system according to the present invention.

The host may be any suitable host cell or virus known to one skilled in the art adapted to be transformed and to manufacture the peptide of the invention, the fusion protein of the invention, or the peptide of sequence (SEQ ID NO: 1). It may be, for example, a eukaryote or prokaryote cell. For example, it may be a eukaryote cell selected from the group comprising COS-7, HEK 293, N1 E115. For example, it may be a prokaryote cell selected from the group comprising TG1 bacteria. It may be also be, for example, a virus, for example a bacteriophage, for example a bacteriophage selected from the group comprising M13 bacteriophage.

Regarding host cells, phages can for example be produced in TG1 (E. coli) bacterial cells. Other strains are readily available to the skilled person, such as the E. coli ER2738 host strain (F′ proA+B+ laclq Δ(lacZ)M15 zzf::Tn10(TetR)/fhuA2 glnV Δ(lac-proAB) thi-1 Δ(hsdS-mcrB)5) of the NEB phage display kit. Other suitable cells may be SS320, or XL1-Blue E. coli cells.

Eukaryote cells are relevant when polypeptides production is sought: for example CHO cells can be used, using conventional methods for polypeptides production well known to the skilled person.

The process for producing/transforming the host may be any process adapted known to one skilled in the art. It may be for example a process involving chemical treatment of bacteria with solutions of metal ions, generally calcium chloride, followed by heating to produce competent bacteria capable of functioning as recipient bacteria and able to take up heterologous DNA derived from a variety of sources. It may be for example a process as disclosed in Sambrook et al., Molecular Cloning: A Laboratory Manual, 2nd edition, (1989) Cold Spring Harbor Laboratory Press, Cold Spring Harbor, N.Y. The process for producing/transforming the host may also use high-voltage electroporation. Electroporation is suitable to introduce DNA into eukaryotic cells (e.g. animal cells, plant cells, etc.) as well as bacteria, e.g., E. coli. as disclosed in Sambrook et al., Molecular Cloning: A Laboratory Manual, 2nd edition, (1989) Cold Spring Harbor Laboratory Press, Cold Spring Harbor, N.Y. One skilled in the art taking into his technical knowledge would adapt/select known processes to obtain the host of the invention.

Another object of the invention is a virus comprising a peptide according to the present invention, a fusion protein according to the present invention, or the peptide of sequence (SEQ ID NO: 1), and/or a nucleic acid sequence according to the present invention or coding for sequence (SEQ ID NO: 1), and/or an expression system according to the present invention.

The virus may be any virus known to one skilled in the art adapted for the expression of peptides or fusion proteins. It may be for example a virus as disclosed in D Bouard et al. “Viral vectors: from virology to transgene expression” Br J Pharmacol. 2009 May; 157(2): 153-165. It may be advantageously a bacteriophage, for example a M13 bacteriophage.

Another object of the present invention is a virus displaying a peptide of the invention and/or a fusion protein of present invention on the surface thereof.

The inventors have surprisingly demonstrated that the present allows to display on viruses functional pentamers consisting of peptides of the invention, which reproduce the structure of the STxB pentamer.

In particular, the inventors have demonstrated that the present invention allows the expression of peptide and fusion protein by the same virus using the expression system of the invention. The proportion of the peptide compared with the fused protein on the virus being 50% each.

The inventions also relates to a virus displaying at its surface a polypeptide of the invention according to any one of the embodiments disclosed herein and/or a fusion protein of the invention according to any one of the embodiments disclosed herein.

Accordingly, the present invention advantageously allows the display of pentameric proteins similar to STxB on the surface of a virus particle such as phage. The pentameric proteins similar to STxB comprise binding sites defined as “binding pockets”, involving hydrogen bonds and hydrophobic stacking interactions between the residues of these confined pockets and the carbohydrate part of glycosphingolipids.

According to the invention, the virus of the invention may form a library of viruses comprising a plurality of virus particles displaying on their surface the peptides of the invention and/or the fusion proteins of the invention.

According to the invention, the virus particles of the library of viruses of the present invention may thus express and display at their surface at least a peptide of the invention, and/or a fusion protein of the invention.

According to the invention, the virus particles of the library of viruses display on their surfaces pentamers of peptides of the invention, such as pentamers of peptides of SEQ ID NO: 1 optionally bound to fusion proteins of the invention.

According to the invention, the virus particles of the library may be bacteriophages (phages). They may for example be M13 bacteriophages.

The invention also relates to a library of viruses, encompassing a plurality of virus of the invention, which are, according to a particular embodiment, phages.

Accordingly, the library of viruses of the invention may be a phage display library.

The invention also relates to a filamentous bacteriophage displaying a polypeptide of the invention according to all aspects described herein and/or a fusion protein of the invention according to all aspects described herein at its surface, the genome of the filamentous bacteriophage comprising a fusion gene, in particular as defined in instant description. It will be understood that a fusion gene is a nucleic acid molecule as defined above and according to any embodiment described herein, which assembles a first and a second nucleic acid molecule as defined in the present description.

The invention also relates to a filamentous bacteriophage encapsulating a nucleic acid molecule or vector according the present description, especially a nucleic acid molecule or vector comprising a fusion gene as defined herein, which displays an STxB-subunit or a variant thereof at its surface.

It will be understood at the expression “displays an STxB-subunit or a variant thereof at its surface” means that at least one STxB-subunits or a variant thereof is displayed, since up to 5 STxB-subunits or variants thereof can be displayed. Accordingly, by “at least one”, it is meant 1, 2, 3, 4 or 5, or a combination of those numbers when a population of bacteriophages is considered.

In another embodiment, the filamentous bacteriophage displaying an STxB-subunit or a variant thereof at its surface, wherein said STxB-subunit has at least one or several of the following properties: said STxB-subunit is functional, and/or properly folded, and/or adopts a pentameric configuration.

The invention also relates to a filamentous bacteriophage displaying at its surface an STxB-subunit or a variant thereof as defined hereafter and herein, the genome of said filamentous bacteriophage comprising a fusion gene as defined above and herein, encompassing, from its 3′ to its 5′ extremities:
a. a first nucleic acid sequence encoding an STxB-subunit monomer or a variant thereof, and
b. a second nucleic acid sequence encoding at least a portion of a pIII filamentous phage coat protein, wherein said fusion gene comprises between the first and second nucleic sequences at least one stop codon.

It will be understood that a filamentous bacteriophage of the invention displays an STxB-subunit or a variant thereof at its surface, through use of a pIII fusion protein. To this end, a nucleic acid sequence of interest, i.e., the first nucleotide sequence, is inserted downstream of the pIII gene in the filamentous bacteriophage genome, which enables the expression of pIII coat protein(s) in a recombinant form, i.e., pIII coat protein(s) carrying a fusion protein. Use of pIII fusion proteins allows the display of up to 5 fusion proteins at the surface of a filamentous bacteriophage.

Said differently, the genome of a filamentous phage of the invention encompasses a first nucleic acid sequence encoding an STxB-subunit monomeric fragment or a variant thereof operably linked and/or fused to a second nucleic acid sequence encoding at least a portion of a pIII filamentous phage coat protein. By “operably linked” in this context, it is meant “joined as part of a same nucleic acid molecule”, i.e., the genome, all parts being preferably suitably positioned and oriented for transcription to be initiated from a promoter, following an open-reading frame that is convenient for implementation of the invention, as discussed herein. The skilled person will appreciate that the junction between the first and second nucleic acid sequences can either be in a form wherein the first and second nucleic acid sequences are physically adjacent to one another, or in a form wherein the first and second nucleic acid sequences are separated by other sequence(s), as commonly implementable by the skilled person given his knowledge.

For instance, a fusion gene as described herein can mean a nucleic acid sequence having several sequences, in particular as identified according to any embodiment herein, covalently linked together and part of a same nucleic acid sequence, but having separate functions or properties. A fusion gene can encompass linker sequences, containing one or more nucleotides. According to a particular embodiment, all sequences constituting the fusion gene are within an open reading frame ensuring proper translation of the fusions gene over the whole length. In particular, the at least one stop codon(s) are within said open reading frame.

As used herein, the expression “filamentous bacteriophage” is construed as a synonym for “filamentous bacteriophage particle”. Also, the expressions “bacteriophage” and “phage” are used interchangeably herein.

By “filamentous bacteriophage”, it is meant a type of bacteriophage, or virus of bacteria, defined by its filament-like or rod-like shape, which contains a genome of single-stranded DNA and infects Gram-negative bacteria. According to a particular embodiment, a filamentous bacteriophage is an Ff phage having a filamentous appearance and being dependent upon a F pilus for bacterial infection. Accordingly, Ff phages are filamentous phages that infect gram negative bacteria, especially E. coli, bearing a F episome.

According to particular embodiment, a filamentous bacteriophage or Ff phage as defined herein is selected among the following phages: f1, fd and M13. All these phages are known to share a genome that is highly homologous, with up to 98% homology or more between them. According to more particular embodiment, a filamentous bacteriophage as defined herein is a M13 phage. The skilled person has ready access from the literature to all the structural features of phages as disclosed herein, including sequences, that are required for carrying out a conventional production of phages.

The most common helper phage is M13KO7, a M13 derivative that carries the mutation Met40Ile in gll, with the origin of replication from P15A and the kanamycin resistance gene from Tn903, both inserted within the M13 origin of replication (Vieira and Messing, 1987 Production of single-stranded plasmid DNA. Methods Enzymol. 1987; 153:3-11). The helper phages thus have a slightly deficient origin of replication that causes less effective replication than phagemids. This process is termed as “phage rescue”. Other helper phages known in the art include R408, VCSM13 (Stratagene), phage vector fd-tet (Zacher et al, Gene, 1980, 9, 127-140).

Phages according to the present invention carry a single-stranded DNA genome encoding several genes, in particular all or a part of the following groups of genes, which are well documented in the literature: (1) genes II, V and X, which encode proteins needed for replication of the phage DNA, (2) genes III, VI, VII, VIII and IX, which encode surface envelope proteins, respectively termed pIII, pVI, pVII, pVIII and pIX, and (3) genes I, IV and XI, which encodes proteins needed for virion assembly. It will be understood that a phage according to the present invention encompasses all features commonly known for phages to be operational in the field of the invention, in particular but not only for phage display purposes. For instance, a phage genomic DNA generally carries an origin of replication (ori) and a “packaging signal” site which initiates virion assembly. Numerous publications are readily available to the skilled person, documenting the common and required features of phages for them to be operational in the field of the present invention. Reference is for example made to N. V. Tikunova and V. V. Morozova, Acta Naturae. 2009 October; 1(3): 20-28. Of note, a pIII sequence is disclosed under database entry NP_510891.1. Fragments of pIII are commonly used.

Therefore, according to a particular embodiment, the pIII filamentous phage coat protein encoded by the fusion gene part of the genome of the filamentous bacteriophage of the invention corresponds to at least a portion or a whole pIII filamentous phage coat protein as found in a filamentous bacteriophage as defined herein, especially those of any one of a f1, fd and M13 phage. The nucleic acid sequences encoding such a pIII phage coat protein or a part of it are readily available to the skilled person (see database entry above).

According to a particular embodiment, the pIII filamentous phage coat protein is from the pIII phage coat protein of bacteriophage M13, as shown in SEQ ID N: 16, i.e., comprises or consists essentially of or consists of said sequence, or a portion of it.

According to a particular embodiment, the second nucleic acid sequence accordingly comprises or consists essentially of or consists of SEQ ID NO: 19, or portion of it.

By “STxB-subunit”, also referred to using the expressions “B-subunit of Shiga toxin” or “STxB protein” or “STxB” herein, it is meant a so-called proteic STxB subunit which is formed by a pentameric assembly of polypeptides of the invention, as defined herein. Said STxB subunit has a pentameric conformation resulting from the non-covalent assembly of five so-called “STxB-subunit monomers”, by analogy with the “monomers” found in the B part of an AB₅ Shiga toxin family member, as defined in the introductory part. According to particular embodiment, an STxB-subunit can be found under the form of an homopentameric assembly of identical B-fragments or monomers, which correspond to polypeptides of the present invention as defined herein.

By “variant of STxB-subunit” it is meant (1) either an homopentameric assembly of B-fragments or monomers as defined above, i.e., polypeptides of the invention, (2) or an STxB-subunit as defined herein, whose constituting monomers have, comprise or consist in an amino acid sequence differing with respect to reference sequence SEQ ID NO: 1, so that the variant amino acid sequence has at least 80%, 81%, 82%, 83% or 84% identity with the sequence of reference. According to a particular embodiment, identity percentages reach 85%, 86%, 87%, 88%, 89%, 90% or more, including 91%, 92%, 93%, 94%, 95%, 96%, 97%, 98% or 99%. In particular embodiments, identity percentages are preferably at least 85% or at least 90% or at least 95% or 99% identity with the sequence of reference.

It will be understood that variant amino acid sequences discussed herein are, according to a particular embodiment, those of the “variant polypeptides” defined in the present description.

According to another embodiment, “variant” also includes STxB-subunit(s), including STxB-subunit(s) whose constituting monomers have a sequence at least 80% identical to a reference sequence as defined in the preceding paragraph, wherein said STxB-subunit(s) are associated, especially coupled, including covalently coupled, or conjugated, with another moiety, especially a moiety that is an active molecule such as a drug, for example a cytotoxic compound, while preserving the functional integrity of said STxB-subunit(s), especially in its(their) structural folding so as to retain a functional activity, according to the definitions provided herein. Such developments are well known in the art. It is acknowledged that, for instance, introduction of a cysteine residue does not perturb the folding neither the binding, while allowing for chemical coupling of the STxB-subunit(s) with active ingredients. Such an active ingredient can for example be auristatin, as previously done by Johannes et al. According to a particular embodiment, said STxB-subunit(s) may be found associated with the natural A-subunit of the Shiga toxin, which naturally dimerizes with STxB pentamers. According to another aspect, the invention also relates to a filamentous bacteriophage displaying at its surface an STxB-subunit or variant thereof that is associated, especially coupled, including covalently coupled, or conjugated, with another moiety, especially a moiety that is an active molecule such as a drug, for use as a medicament.

By “STxB-subunit monomer (or fragment)”, also referred to using the expressions “monomer (or fragment) of B-subunit of Shiga toxin” or “STxB protein monomer (or fragment)” or “STxB monomer (or fragment)” herein, it is meant a polypeptide as found in the B part of an AB₅ Shiga toxin family member, as defined above and herein. By “variant of an STxB-subunit monomer”, it is meant an STxB-subunit monomer comprising or consisting of an amino acid sequence differing with respect to a reference sequence SEQ ID NO: 1, so that the variant amino acid sequence has at least 80%, 81%, 82%, 83% or 84% identity with the sequence of reference. According to a particular embodiment, identity percentages reach 85%, 86%, 87%, 88%, 89%, 90% or more, including 91%, 92%, 93%, 94%, 95%, 96%, 97%, 98% or 99%. In particular embodiments, identity percentages are preferably at least 85% or at least 90% or at least 95% or 99% identity with the sequence of reference.

It will be understood that variant amino acid sequences discussed herein are, according to a particular embodiment, those of the “variant polypeptides” defined in the present description.

Accordingly, according to a particular embodiment a first nucleic acid sequence encoding an STxB-subunit monomer or a variant thereof is as defined herein when nucleic acid molecules of the inventions are described.

By extension, as defined herein, the definition provided for a “variant” also applies to nucleic acid molecules defined herein, including nucleic acid molecules encoding amino acid sequence(s) as defined herein, or as described herein.

Modification(s) defining variant amino acid sequences can independently be deletion(s), including especially point deletion(s) of one or several amino acid residue(s) or can be substitution(s), especially conservative substitution(s) of one or several amino acid residue(s).

The definitions provided herein with respect to the terms or expressions “one or several”, “identity”, “Identity percentages”, also apply.

According to the invention, a fusion gene integrated in the genome of a filamentous bacteriophage of the invention comprises between the first and second nucleic acid sequences at least one, in particular one or two, stop codon(s), also termed termination codon(s) or nonsense codon(s) herein. As defined herein, a stop (or termination, or nonsense) codon is a nucleotide triplet that, when found in a messenger RNA obtained from the departure encoding DNA, signals a termination of translation of the RNA sequence into a protein.

Accordingly, in a particular embodiment, the genome of a filamentous bacteriophage of the invention is engineered so that (at least one) stop codon is inserted or found between said first and second nucleic acid sequences. According to another embodiment, the sequence of the genome of a filamentous bacteriophage of the invention comprises between said first and second nucleic acid sequences added and/or substituted and/or suppressed nucleotide(s) so that at least one stop codon is inserted or found between said first and second nucleic acid sequences.

A stop codon as defined herein can be any stop codon known in the art to cause premature termination of a translation into a protein or functional polypeptidic sequence. According to particular embodiments, the stop codon is selected among DNA stop codons TAG, TAA and TGA. Said otherwise, the stop codon corresponds to a DNA triplet nucleotide sequence (codon) encoding an mRNA suppressible terminator codon selected from UAG, UAA and UGA (RNA stop codons).

According to a particular embodiment, the stop codon results from a so-called “amber mutation” within the fusion gene, i.e., results from a nonsense mutation that changes a sense codon (specifying an amino acid) into a translational stop codon, causing premature termination of the polypeptide chain during translation. The term “amber”, as in particular used herein, refers to a mutation causing the termination of translation through this mechanism, or to the codon, which may in this case be a TAG codon (corresponding to a “UAG” RNA codon).

Alternatively or cumulatively, a stop and/or amber codon can be inserted within the fusion gene sequence during fusion gene engineering, through techniques well known in the art. In this context, reference is also made to the experimental section herein. For example, commercially available phagemids include, within their sequence, inserted stop codons between a foreign protein gene and the protein III gene.

From the preceding, it will be understood that the fusion gene integrated in the genome of a filamentous bacteriophage of the invention has a structure which comprises, from its 3′ to its 5′ extremities, (1) at least one, in particular one, first nucleotide sequence as defined herein, (2) at least one, in particular one, stop codon as defined herein and (3) a second nucleotide sequence as defined herein, in the order (1), (2) and (3).

Nevertheless, the skilled person will readily appreciate that the sequence of a genome of a filamentous bacteriophage of the invention, including within its integrated fusion gene sequence, may encompass other sequences conventionally known by the skilled person as useful in the field of the invention, such as for example, linkers or tag encoding nucleic acid sequence(s).

According to a particular aspect, a filamentous bacteriophage of the invention displays at its surface an STxB-subunit or a variant thereof, as defined herein.

According to a particular, not exclusive, embodiment, the displayed STxB-subunit or a variant thereof is functional. By “functional”, it is meant that said STxB-subunit or a variant thereof as defined herein has a conformation that enables retaining a binding capacity of the STxB-subunit or a variant thereof to a target, i.e, has measurable functional target binding activity. According to a particular embodiment, said target is a glycosphingolipid (GSL). Accordingly, according to a particular embodiment, the displayed STxB-subunit or a variant thereof specifically binds glycosphingolipids (GSLs), especially GSLs expressed at the surface of a cell or tissues, including neoplasic of carcinogenic cells or tissues. Differently said, and according to a particular embodiment, a filamentous phage of the invention retains the functional capacity to recognize, or bind, or specifically bind, one or several GSL(s). Examples of GSLs known to the skilled person are readily available from the literature. For example it may be GSLs as disclosed in Ronald L Schnarr et. al, Essentials of Glycobiology. 2^{nd} edition, Chapter 10 Glycosphingolipids. It may be for example a GSL selected from the group comprising Gb3, Globo H, isoGb4, fucosyl GM1, GM2, neuAcGM3, GD1a, GD2, GD3, or as defined herein.

According to a particular embodiment, the functional properties of a filamentous phage of the invention retaining binding capacity to a target as defined herein through the displayed STxB-subunit or a variant thereof, are tested by Western blot analysis to assess the proper display of STxB or variants in fusion with pIII (see in particular Experimental section herein for guidance).

Other means for assessing binding can be:
FACS (Fluorescence-activated cell sorting) experiments: phage display STxB or variants are putted in contact with cells presenting the GSL of choice. After staining with anti-PIII antibody and appropriate fluorescent secondary antibody, the fluorescent intensity on cells is measured by FACS in comparison with a control (i.e., phages that do not bind the cells), and/or
Immunofluorescence microscopy (binding of phages on seeded cells, antibody staining, epifluorescence microscopy), and/or
Assessment of the binding on liposomes containing GSL (phages are mixed with magnetic liposomes, after washes recruitment of liposomes on a magnet, elution and loading on a gel, similar to a pull down experiment—see Experimental section herein).

According to particular embodiments, functionality is present when the binding affinity of an STxB-subunit or a variant thereof displayed at the surface of the filamentous phage of the invention and its target, which is a GSL as defined herein, can be demonstrated.

According to another, not mutually exclusive, particular embodiment, the displayed STxB-subunit or a variant thereof is deemed to be properly folded. “By properly folded”, it is meant that the displayed STxB-subunit or a variant thereof adopts a quaternary folding that is substantially similar to that of STxB-subunits as found in their natural environment, in particular soluble form, and/or retains the functional properties discussed above.

According to another, not mutually exclusive, particular embodiment, the displayed STxB-subunit or a variant thereof adopts a pentameric configuration, such as a pentameric conformation resulting from the non-covalent assembly of five so-called “STxB-subunit monomers” as found in the B part of an AB₅ Shiga toxin family member, as defined above and herein. It will be appreciated that in this context an STxB-subunit may be found under the form of an homopentameric assembly of identical B fragments or monomers.

According to another aspect, it will be appreciated that the inventors defined that a proper folding of an STxB-subunit or a variant thereof displayed at the surface of a phage of the invention, could be achieved when one STxB monomer (or variant thereof) in fusion with a pIII page coat protein assembled with four other STxB monomers found under a non-fused, i.e., “free”, form (see FIG. 11B). This configuration further enables display of one to five STxB-subunits or variants thereof at the surface of a phage particle.

According to a particular embodiment, the STxB-subunit or variant thereof displayed at the surface of a phage of the invention adopts the following, especially pentameric, structure: one STxB monomer or variant thereof is fused with a pIII phage coat protein, said monomer or variant thereof being assembled with four other STxB monomer or variant thereof, which are found under a non-fused form, i.e., a free form according to a definition adopted herein.

It can be appreciated that inventors' experiments assess that an embodiment corresponding to five STxB monomers or variants thereof fused with a pIII phage coat protein does not enable proper display of a pentameric assembly at the surface of a phage (FIG. 11A rationale).

Nonetheless, according to another particular embodiment, the STxB-subunit or variant thereof displayed at the surface of a phage of the invention adopts the following, especially pentameric, structure: One STxB monomers or variant thereof is respectively fused with a pIII phage coat protein, and this monomer or variant thereof is assembled with four other STxB monomer or variant thereof, which are found under a non-fused form, i.e., a free form according to a definition adopted herein.

A non-fused form means that said monomers are not fused with any phage protein, especially phage coat protein, in particular pIII page coat protein. According to a particular embodiment, a non-fused (or free) form is a topological form that is substantially the native or natural form of an STxB monomer or variant thereof.

According to a particular embodiment, a displayed STxB-subunit or variant thereof having the configuration of the paragraphs above is further defined as functional and/or properly folded and/or adopts a pentameric configuration, according to the definitions provided above, i.e, it retains functional activity, especially target, in particular GSL(s), binding activity when found as a single monomer fused to a pIII phage coat protein, assembled with four other STxB monomers. According to a particular aspect, such a displayed STxB-subunit or variant thereof is defined as retaining a functional conformation.

According to a particular embodiment, a filamentous phage defined herein displays between 1 to 5 STxB-subunit(s) or variant(s) thereof at its surface, in particular 1, 2, 3, 4 or 5 STxB-subunit(s) or variant(s) thereof.

According to a particular embodiment, the filamentous bacteriophage is an isolated and/or recombinant bacteriophage.

The invention also relates to a filamentous phage, which displays a polypeptide that is a STxB-subunit or variant thereof, the latter of which being selected among the polypeptides defined herein, or functional equivalent(s) thereof. According to a particular embodiment, the displayed STxB-subunit or variant thereof is functional and/or adopts a pentameric configuration at the surface of the phage.

According to a further characteristic, the displayed STxB-subunit or variant thereof has binding capacity to a target selected among those defined in the present description.

In a particular embodiment, the displayed STxB-subunit or variant thereof is under the form of one STxB monomer or variant thereof in fusion with a pIII page coat protein, the STxB monomer or variant thereof being assembled with four other free STxB monomers.

The invention also relates to nucleic acid molecules as defined herein, in particular nucleic acid constructs suitable as means for cloning or expressing nucleic acid molecules of the present disclosure, such as vectors, in particular plasmids.

The invention therefore also concerns a vector, in particular a plasmid comprising at least one nucleic acid molecule as defined herein.

A plasmid or vector can be used either for cloning, for transfer or for expression purposes.

It will be understood that in the context of the present invention, a plasmid of particular interest is a plasmid suitable for the cloning of the nucleic acid molecule it contains. Such a cloning plasmid may be a bacterial plasmid, encompassing an origin of replication and multiple restriction enzyme cleavage sites allowing the insertion of a transgene insert (transcription unit), e.g., a nucleic acid molecule of the invention as defined herein, in particular a nucleic acid molecule comprising a first and a second nucleic sequence as disclosed according to any embodiment herein, and a stop codon in-between, or a fragment thereof.

However, according to another particular embodiment, a plasmid of the invention is suitable for the expression of the nucleic acid molecule it contains. Such an expression plasmid, also termed expression vector herein, or expression construct, generally contains a promoter sequence, a transcription terminator sequence, and a transgene insert (transcription unit), e.g., a nucleic acid molecule as defined herein, or a fragment thereof. An expression vector may also contain an enhancer sequence which increases the amount of protein or RNA produced.

A plasmid may be found as a single stranded DNA molecule or a double-stranded DNA molecule.

Particularly encompassed plasmids are phagemids and phage vectors.

The invention therefore also relates to a phagemid comprising a nucleic acid molecule as defined herein. A phagemid (or phasmid) is a plasmid that contains an f1 origin of replication from an f1 phage, which can be used as a type of cloning vector in combination with a so-called “helper” virus (especially helper phage) or appropriate packaging cell line. A phagemid can either be replicated as a plasmid or be packaged as single stranded DNA in viral particles, especially a filamentous bacteriophage of the invention. Phagemids contains at least an origin of replication (ori) for double stranded replication, as well as an f1 ori to enable single stranded replication and packaging into phage particles. The skilled person can readily select an appropriate phagemid structure for the purpose of implementing the present invention.

According to particular embodiments of the invention, a phagemid of the invention encompasses one or several of the following, according to all possible combinations: a selectable marker, a ColE1 origin, an f1 origin, transcription terminator(s), promoter(s), ribosomal binding site(s), leader sequences(s), and a nucleotide sequence as defined herein that enables the display of an STxB-subunit or a variant thereof as defined herein, at the surface of a filamentous bacteriophage of the invention.

According to a particular embodiment, a phagemid of the invention comprises all or a part of the sequence of the phagemids disclosed in the Experimental section (so-called “pHEN2_STxB phagemids”), the sequence of which is provided herein under SEQ ID NO: 35 (4804) nucleotides).

According to a particular embodiment, a nucleic acid molecule of the invention in which items (1), (2) and (3) described above are found, is embedded within a pHEN2 phagemid, the structure of which readily accessible to the skilled person in the art, such a phagemid being of common use.

The invention also relates to a phage vector comprising a nucleic acid molecule of the invention as defined herein.

The invention also relates to a filamentous bacteriophage encapsulating a nucleic acid molecule or vector of the invention as defined herein, in particular a nucleic acid molecule as embedded within a phagemid according to any one of the embodiments disclosed herein. According to a particular aspect, such an encapsulation enables the display of an STxB-subunit or a variant thereof at the surface of the bacteriophage, due to the presence of a construct comprising items (1), (2) and (3), when the filamentous bacteriophage is allowed to replicate under appropriate conditions, i.e., in particular within a nonsense suppressor strain/cell line. A nonsense suppressor strain display base substitution mutations in the DNA corresponding to the anticodon of a tRNA that cause the anticodon to pair with one of the termination (or “nonsense”) codons, UAG (Amber), UAA (Ochre), or UGA (Opal). (See http://ecoliwiki.net/colipedia/index.php/Nonsense_suppressor and http://www.sci.sdsu.edu/˜smaloy/MicrobialGenetics/topics/rev-sup/nonsense-suppressors.html for examples of nonsense suppressors produced by single base substitutions in E. coli, the latter page being provided for illustration and incorporated herein by reference.)

The inventors have also demonstrated that the presence of a stop codon was particularly useful so that STxB-subunit or a variant thereof can be displayed at the surface of a phage according to the scheme provided in FIG. 11B. The experimental section shows how to take advantage of the presence of a stop codon to allow the production of both free STxB-subunit monomers or variant thereof and a form of STxB-subunit monomers or variant thereof that is fused with a pIII phage coat protein is enabled. Fused and free forms of STxB-subunit monomers or variant thereof can assemble in the periplasm of cells used for phage production/replication, so that the resulting phage displays a functional STxB-subunit or a variant thereof at its surface, according to the definitions provided herein.

Another object of the invention is a cell or a cell host, especially a bacterial cell or a bacterial cell host comprising a nucleic acid molecule, vector, plasmid, phagemid or filamentous phage according to the invention, as disclosed in any one of the embodiments described herein.

According to a particular embodiment, such a host cell is an E. coli cell, especially a E. coli cell capable of packaging the phagemids disclosed herein into filamentous phage of the invention as disclosed herein, in particular using the protocols and methods described herein.

The invention also relates to a composition comprising or consisting of nucleic acid molecule, vector, plasmid, phagemid or filamentous phage according to the invention, or cells comprising the same, as disclosed in any one of the embodiments described herein.

The invention also relates to a library (or collection) of nucleic acid molecules as disclosed herein, especially a library encompassing variants of nucleic acid molecules as disclosed herein.

The invention also relates to a library of vectors, especially plasmids or phagemids as disclosed herein, especially a library encompassing variants of vectors, especially plasmids or phagemids as disclosed herein.

The invention also relates to a library of bacterial cells, especially bacterial cells comprising nucleic acid molecules, or plasmids or phagemids, or filamentous phages as disclosed herein, especially a library encompassing variants of nucleic acid molecules, or plasmids or phagemids, or filamentous phages as disclosed herein.

The invention also relates to a library of filamentous phages, especially a library encompassing variants filamentous phages as disclosed herein. Such a library is commonly obtained for phage display.

The invention indeed also provides for a method of production of filamentous phages as disclosed herein, or a library thereof, comprising the steps of:
a) Introducing one or several phagemid(s) as disclosed herein into bacterial cell(s) as disclosed herein, and
b) Culturing said bacterial cell(s) of step (a), optionally in the presence of helper phage(s) as commonly known and practice in the art, in particular under conditions enabling production of phages of the invention displaying STxB-subunit(s) or variant thereof at the surface of the filamentous phages, or a library thereof, and
c) Optionally, recovering (harvesting) the produced filamentous phage(s) or library thereof and/or isolating a particular species of produced filamentous phages or library thereof.

According to a particular embodiment, the method of production of filamentous phages of the invention results in the production of a library (or collection) of filamentous phages as disclosed herein, especially a library encompassing phage variants as disclosed herein, when a library (collection) of phagemids, especially encompassing phagemids variants as disclosed herein, is used.

The Experimental section herein provides specific protocols in this respect, the features of which are part of the present invention, according to all possible combinations thereof, with said features being, according to particular embodiments, isolated from each other.

The invention also relates to filamentous phage(s) as disclosed herein, or a library thereof, obtainable through a method of production as disclosed herein.

It will be understood that filamentous phages of the invention or libraries encompassing them are conceivably relevant tools for screening putative targets retaining a binding capacity to an STxB-subunit or a variant thereof as disclosed herein, especially using phage display. As detailed herein, putative STxB-subunits or variant thereof targets encompass glycosphingolipids (GSLs), since Gb3 is the natural binder for most known STxB proteins. Accordingly, putative targets encompass commonly known glycosphingolipids and variants thereof, as in particular described herein, especially above in Table 2, because the inventors rationale and consensus sequence design took into account their knowledge and data about conformational requirements for binding other GSLs than Gb3.

As shown in the Experimental section herein, the inventors confirmed that displaying a STxB moiety, which is an assembly of monomers of SEQ ID NO: 1 on an M13 bacteriophage specifically drives its binding on Gb3⁺ cells. Such a display can conveniently be exploited in the context of screening libraries in which the STxB gene is systematically mutated to obtain variants that may gain binding activity against glycosphingolipids to which natural STxB proteins do not bind naturally.

It will be understood that such variants that may gain binding activity against glycosphingolipids to which natural STxB proteins do not bind naturally are appropriately encompassed within the definitions of variants provided herein.

The preparation of phages display libraries of peptides and proteins is now well known in the art. These methods generally require transforming cells with phagemid vector DNA to propagate the libraries as phage particles having one or more copies of the variant peptides or proteins displayed on the surface of the phage particles. See, for example Bonnycastle et al., J. Mol. Biol., (1996), 258:747-762; and Vaughan et al., Nature Biotechnology (1996), 14:309-314.

According to the invention, the method of producing phage display libraries may comprise the following steps:

a) infecting bacteria with phages comprising a phagemid expressing a nucleic acid of the invention or transforming bacteria with a phagemid expressing a nucleic acid according to the invention,

b) optionally infecting the infected or transformed bacteria with an amount of helper phage encoding a phage coat protein sufficient to produce recombinant phagemid particles which display the peptide of the invention and/or fusion protein of the invention on the surface of the particles,

c) culturing the infected or transformed bacteria under conditions suitable for forming a library of phage displaying expressing peptide of the invention or fusion protein of the invention.

In the method the bacteria may be any bacteria known to one skilled in the art that could be infected with phages and adapted. It may be for example Escherichia coli, for example TG1, SS320, ER2738, or XL1-Blue E. coli.

According to the present invention, the phage comprising a phagemid expressing a nucleic acid of the invention is as defined above.

According to the present invention, step a) of infection may be carried out with any method and/or under any conditions adapted known to one skilled in the art. For example, the skilled person taking into consideration his technical knowledge could select and/or adapt the multiplicity of infection conditions with regards to the phage and the bacteria used.

According to the present invention, the preparation of the phage display library may comprise infecting bacteria with a phagemid vector according to the invention.

One skilled in the art, taking into consideration his technical knowledge is aware of several ways of infecting of bacteria and would be able to adapt such processes to the present invention.

According to the present invention, the preparation of the phage display libraries may comprise transforming bacteria with a phagemid vector according to the invention.

One skilled in the art, taking into consideration his technical knowledge knows of several processes for transforming bacteria, and would be able to adapt such processes to the present invention. For example, the process for transforming may be a method involving chemical treatment of bacteria with solutions of metal ions, generally calcium chloride, followed by heating to produce competent bacteria capable of functioning as recipient bacteria and able to take up heterologous DNA derived from a variety of sources, a method using high-voltage electroporation for example as disclosed in Dower et al., 1988, Nucleic Acids Research, 16:6127-6145.

According to the present invention, the optional step b) of infection of the infected or transformed bacteria of step a) with an amount of helper phage may be carried out with any method and/or under any adapted conditions known to one skilled in the art. For example, the skilled person taking into consideration his technical knowledge would select and/or adapt the multiplicity of Infection, the multiplicity of infection techniques with regards to the helper phage and the bacteria used.

According to the present invention, the helper phage may be any helper phage known to one skilled in the art and/or commercially available and adapted to the present invention. It may be for example M13KO7 Helper Phage.

One skilled in the art, taking into consideration his technical knowledge would be able to select the helper phage adapted to the phages and/or phagemids and/or bacteria used.

Advantageously, the phagemid vector used in the process of preparing the phage display library may comprise a nucleic acid coding for a fusion protein of the invention in which the end of the sequence coding for the peptide of the invention or for sequence SEQ ID NO: 1 may comprise stop codon.

Advantageously, the phagemid vector used in the process of preparation may be the pHEN2 phagemid comprising of a nucleic acid coding for a fusion protein of the invention in which the end of the sequence coding for the peptide of the invention or for sequence SEQ ID NO: 1 may comprise an amber stop codon.

Accordingly, the present invention also provides a library of phage particles comprising a plurality of phages, the phage particles displaying a peptide of the invention and/or of SEQ ID NO: 1 and/or fusion protein of the invention on the surface thereof, wherein each fusion protein comprises at least a portion of a protein III phage coat protein and a peptide of the invention or of SEQ ID NO: 1, wherein said phage coat protein is fused to the N-terminus of said peptide of the invention or of SEQ ID NO: 1.

Accordingly, the phages of the library of phages advantageously display on their surfaces pentameric proteins similar to STxB pentamer which is comprises fifteen binding sites per pentamer. Those binding sites can be called “binding pockets”. Through hydrogen bonding and hydrophobic stacking interactions, the residues of the confined pocket are able to receive and link with the carbohydrate moieties of glycospingolipids.

Accordingly, the viruses of the library of phages advantageously display on their surfaces pentameric proteins similar to STxB pentamer which is composed of fifteen binding sites per pentamer. Those binding sites are defined as “binding pockets”, involving hydrogen bonds and hydrophobic stacking interactions between the residues of this confined pocket and the carbohydrate part of glycolipids

According to the invention, the phages of the invention may also further comprise a supplemental phagemid comprising a nucleic acid coding for a second fusion protein comprising a polypeptidic structure fused to a coat protein of a phage.

According to the invention the polypeptidic structure may be an antigen or epitope. It may be for example any antigen or epitope known to one skilled in the art that could be targeted to antigen presenting cells.

According to the invention, the supplemental phagemid may be any phagemid known to one skilled in the art adapted to the expression of fused protein as mentioned above.

In the present, “presenting cells” may be any presenting cells known to one skilled in the art. It may be for example cells selected in a group comprising T lymphocytes, dendritic cells, macrophages Langerhans cells and the like.

According to the invention the major coat protein of the second fusion protein may be a pVIII protein phage coat protein.

According to the invention, the phages of the invention may also be used for phage display screening.

According to the invention, the phages of the invention may also be used to target compounds to a particular tissue or cells expressing glycosphingolipids. For example the phages of the invention may allow to target to an antigen presenting cell expressing glycosphingolipids a compound which may be constituted by or may comprise a polypeptidic structure, such an antigen or epitopes thereof.

The inventors also provides a process for producing peptides of the invention and in particular a process for producing peptides that still have the pentameric structure of STxB and that could specifically bind and target glycosphingolipids.

The inventions also relates to a method for producing a polypeptide or a fusion protein as defined herein by genetic recombination using a nucleic acid molecule or an expression system of the present disclosure.

The present invention also relates to a method for producing peptides of the present invention and/or fusion proteins comprising the culturing of a host of the present invention comprising a nucleic acid sequence according to the invention or an expression system according to the invention.

One skilled in the art, taking into consideration his technical knowledge would know the range of appropriate culture conditions, for example the culture medium used, the temperature, depending on the host.

The method for producing the peptides and/or fusion protein according to the invention may also use any adapted host transformed for a production by genetic recombination in accordance with the present invention.

The method for producing the peptides and/or fusion protein according to the invention may also comprise a step of recovering or isolating peptides of the invention and/or fusion proteins according to the invention.

The recovery step or isolating step can be carried out by any means known to one skilled in the art. It may, for example, involve a technique chosen from electrophoresis, molecular sieving, ultracentrifugation, differential precipitation, for example with ammonium sulfate, by ultrafiltration, membrane or gel filtration, ion exchange, elution on hydroxyapatite, separation by hydrophobic interactions, or any other known means.

One skilled in the art, taking into consideration his technical knowledge would be able to select the recovery step or isolating step depending on the host and the peptide or fusion protein that is being produced.

The inventors have also demonstrated that the peptide of the invention may be used as a component of a host or be a component of a host, for example a bacteriophage. In particular the inventors have surprisingly demonstrated that the peptide of the invention or of sequence SEQ ID NO: 1 or fusion protein of the invention may be expressed at the surface of a host and the host can be used directly for detecting molecules, for example glycosphingolipids, in a sample.

As mentioned above, the inventors have surprisingly demonstrated that the present invention allows advantageously to display on the surface of viruses, for example phages, pentameric structures formed with peptide/fusion proteins according to the invention, which have a structure similar to the STxB pentamer composed of fifteen binding sites per pentamer. These pentameric structures contain binding sites or “binding pockets”, which enable the binding of glycolipids such as glycosphingolipids, through hydrogen bonding and hydrophobic stacking interactions between the residues of this confined pocket and the carbohydrate part of glycosphingolipids.

Accordingly, the viruses of the library of viruses allow advantageously to display on their surfaces pentameric proteins similar to STxB pentamers, which is composed of fifteen binding sites per pentamer. Those binding sites are defined as “binding pockets” which are able to bind to glycosphingolipids as mentioned above.

According to another aspect, a virus of the invention displaying a peptide and/or a fusion protein on the surface thereof can be used to detect glycosphingolipids in a sample.

The inventors have also demonstrated that the virus of the invention displaying a peptide and/or a fusion protein on the surface thereof may be used to identify and/or select peptides of the invention that bind to particular glycosphingolipids. In particular, the inventors have demonstrated that the phage display library of the invention may be used to identify and/or select peptides of the invention that bind to particular glycosphingolipids.

Accordingly, another object of the present invention is the in vitro use of a polypeptide and/or of a fusion protein as defined herein, or a host expressing the peptides of the invention or a virus of the invention or a library of viruses of the invention for detecting molecule(s), which can be is a glycosphingolipid(s), and/or a cell in a sample

In the present the sample may be a biological sample. The biological sample may be any biological sample known to one skilled in the art. The biological sample may for example be a liquid or solid sample. According to the invention, the sample may be any biological fluid, for example it can be a sample of blood, plasma, serum, urine, tissue, for example muscle, or a sample from a tissue biopsy.

In the present the molecule may be any glycosphingolipid known to one skilled in the art. For example it may be a glycosphingolipid as disclosed in Ronald L Schnarr et. al, Essentials of Glycobiology. 2nd edition, Chapter 10 Glycosphingolipids. It may be for example a glycosphingolipid selected from the group comprising Gb3, Globo H, isoGb4, fucosyl GM1, GM2, neuAcGM3, GD1a, GD2, GD3, or any glycosphingolipid especially as defined and/or described herein.

Thus the peptides of the invention and/or host expressing the peptide according to the invention and/or viruses of the invention, and/or phage display of the invention may be also used in an in vitro or in vivo imaging method.

In the present the in vitro or in vivo imaging method may be any method known to one skilled in the art in which a peptide or host cells expressing a peptide could be used. For example in vivo imaging methods may be selected from the group comprising Single Photon Emission Computed Tomography (SPECT), Positron Emission Tomography (PET), Contrast enhanced ultrasound imaging, and Magnetic Resonance Imaging (MRI) by using for example mangradex nanoparticles.

In the present, when peptides according to the invention and/or virus of the invention, and/or phages of the invention is(are) used in detection imaging methods, the peptide of the invention or fusion peptide may be labelled and/or tagged. For example the peptide may be tagged with any tag adapted and known to one skilled in the art. It may for example be a tag selected from the group comprising biotin, fluorescent dyes for example rhodopsine, alexa-Fluor, nanogold coated ligands, carbon-black coated ligands, mangradex, or a fluorescent ligand.

For example the peptides and/or fusion protein may be labelled and/or tagged with a compound selected from the group comprising radioactive molecules, for example comprising radioactive atoms for scintigraphic studies such as ¹²³I, ¹²⁴I, ¹¹¹In, ¹⁸⁶Re, ¹⁸⁸Re, fluorochromes.

One skilled in the art taking into his technical knowledge knows various methods of labelling peptides and proteins and would select and/or adapt such processes to the peptides and/or fusion proteins of the invention.

The inventors have also demonstrated that the peptides of the invention or of sequence (SEQ ID NO: 1) allow to bind and target glycosphingolipids and thus could be used to detect any change in the GLYCOSPHINGOLIPID expression patterns. In particular, pathological situations may have an effect on the pattern expression of glycosphingolipids and could be considered as biomarker of disease.

Thus the peptides of the invention invention or of sequence (SEQ ID NO: 1) and/or host expressing the peptide according to the invention invention, fusion protein of the invention or peptide of sequence (SEQ ID NO: 1) may be used in an in vitro or in vivo diagnostic method.

In particular, the peptides of the invention invention or of sequence (SEQ ID NO: 1) and/or host expressing the peptide according to the invention invention, fusion protein of the invention or peptide of sequence (SEQ ID NO: 1) may be used in an in vitro method for detecting a disease in which glycosphingolipids are misregulated.

In the present invention the in vitro or in vivo diagnostic method may be any method known to one skilled in the art in which a peptide or host cells expressing a peptide could be used. For example in vivo diagnostic method may be selected from the group comprising Single Photon Emission Computed Tomography (SPECT), Positron Emission Tomography (PET), Contrast enhanced ultrasound imaging, and Magnetic Resonance Imaging (MRI) by using for example Mangradex nanoparticles.

In the present, the term “individual” means a mammal selected from the group consisting of Monotremata, Didelphimorphia, Paucituberculata, Microbiotheria, Notoryctemorphia, Dasyuromorphia, Peramelemorphia, Diprotodontia, Tubulidentata, Sirenia, Afrosoricida, Macroscelidea, Hyracoidea, Proboscidea, Cingulata, Pilosa, Scandentia, Dermoptera, Primates, Rodentia, Lagomorpha, Erinaceomorpha, Soricomorpha, Chiroptera, Pholidota, Carnivora, Perissodactyla, Artiodactyla and Cetacea. It may be, for example, a human or an animal.

In the present, the biological sample may be obtained from mammals, for example a mammal selected from the group consisting of Monotremata, Didelphimorphia, Paucituberculata, Microbiotheria, Notoryctemorphia, Dasyuromorphia, Peramelemorphia, Diprotodontia, Tubulidentata, Sirenia, Afrosoricida, Macroscelidea, Hyracoidea, Proboscidea, Cingulata, Pilosa, Scandentia, Dermoptera, Primates, Rodentia, Lagomorpha, Erinaceomorpha, Soricomorpha, Chiroptera, Pholidota, Carnivora, Perissodactyla, Artiodactyla and Cetacea. It may be, for example, a human or an animal.

In the present the biological sample may be any biological sample known to one skilled in the art. The biological sample may for example be a liquid or solid sample. According to the invention, the sample may be any biological fluid, for example it can be a sample of blood, plasma, serum, urine, tissue, for example muscle, or a sample from a tissue biopsy.

In the present “disease/condition in which glycosphingolipids are misregulated” means any condition/disease known to one skilled in the art in which GSLs are misregulated. It may be for example a cancer, a tumor.

Such a disease/condition can be selected among: ovarian cancer, breast carcinoma, Colon cancer, Gastric adenocarcinoma, Burkitt's lymphoma, colon carcinoma, melanoma, small cell lung cancer (SCLC), renal carcinoma, Neuroblastoma, Cervical carcinoma, Glioblastoma, renal carcinoma, Glioma, Retinoblastoma, Neuroectodermal cancer, non-small cell lung cancer (NSCLC), Wilms tumor, Osteosarcoma, and t-All condition.

In the present cancer may be any cancer known to one skilled in the art. It may be for example any disease involving abnormal cell growth with the potential to invade or spread to other parts of the body. It may be for example cancer of any organ or tissue of a human or of an animal. It may be for example a cancer selected from the group comprising lung, liver, eye, heart, lung, breast, bone, bone marrow, brain, head & neck, esophageal, tracheal, stomach, colon, pancreatic, cervical, uterine, bladder, prostate, testicular, skin, rectal, and lymphomas.

In the present “tumor” refers to an abnormal growth of tissue resulting from an abnormal multiplication of cells. A tumor may be benign, premalignant, or malignant (i.e. cancerous). A tumor may be a primary tumor, or a metastatic lesion.

The inventors have also shown that the peptides of the invention or of sequence SEQ ID NO: 1 can bind and target glycosphingolipids expressed at the surface of cancerous cells, and that the means defined herein can accordingly be used to detect/target cells expressing glycosphingolipids.

Another object of the present invention is the in vitro use of the peptide of the invention for detecting cancerous cells, in a sample.

In the present the peptide is as defined above. In particular, the peptide may be labeled or tagged as mentioned above.

The present invention also refers to an in-vitro method for determining the specificity of virus of the present invention to glycosphingolipids.

In particular, the in-vitro method for determining the specificity of a virus of the present invention to glycosphingolipids comprising the following steps:

a) Putting into contact a virus of the present invention and a support comprising glycosphingolipids on its surface,

b) Incubating the virus and the support comprising glycosphingolipids present on its surface to let the virus bind to the glycosphingolipids,

c) Washing the incubated surface to eliminate the non-bounded virus, and

d) Recovering virus bounded to the glycosphingolipids.

According to the invention, step a) of the method for determining the specificity of virus may be carried out in a solution, for example a liquid solution. The solution may be any solution known to a person skilled in the art. It may be, for example, a culture medium, such as a eukaryotic and/or prokaryotic cell culture medium, a buffer medium, for example any buffer medium known to a person skilled in the art, for example a commercially available buffer medium like phosphate buffered saline (PBS).

According to the invention, the support comprising glycosphingolipids on its surface may be any known to one skilled in the art and adapted, provided that the glycosphingolipids are not structurally or biologically modified.

According to the invention the glycosphingolipids may be incorporated or fixed on the surface of the support, or provided as a layer at the surface of, for example, cells.

According to the invention the support may be a biological or artificial support. It may be for example a cell expressing a particular glycosphingolipid, for example it may be any cell known to one skilled in the art adapted to express glycosphingolipids. It may be for example a CHO cell, transformed with an expression vector to express a particular glycosphingolipid. One skilled in the art taking into consideration his technical knowledge knows processes for transforming cells. One skilled in the art knows the nucleic acid sequence coding for glycosphingolipids which could be incorporated into an expression vector. It may also be any cells line that stably expresses glycosphingolipids.

The support may be also an artificial support, for example a supported lipid bilayers, a tethered bilayer lipid membrane, unilamellar vesicles or liposomes.

According to the invention, the supported lipid bilayers, tethered bilayer lipid membranes, unilamellar vesicles or liposomes may be charged with a controlled amount of glycosphingolipids. For example, the supported lipid bilayers may be liposomes prepared according to the following process:

i) mixing lipids with glycosphingolipids,

ii) drying to remove solvent,

iii) resuspending the dried mix with a buffer at a temperature over the melting temperature of said lipids to obtain a preparation,

iv) freeze and thaw the preparation obtained in step iii) to obtain homogeneous preparation comprising liposomes, and

v) Extrusion of homogeneous preparation of step iv) to obtain homogeneously sized liposomes.

According to the invention, the lipids used in steps i) may be any lipids known to one skilled in art adapted to form lipid bilayers. It may be for example a lipid selected from the group comprising glycerophospholipids, for example phosphatidylethanolamine (cephalin) (PE), Phosphatidylcholine (lecithin) (PC), Phosphatidylserine (PS) 1,2-Dioleoyl-sn-glycero-3-phosphocholine (DOPC).

According to the invention, the lipids may further comprise cholesterol.

According to the invention, the lipids may further comprise modified lipids, for example fluoroscently labelled lipids, chemically modified lipids, for example biotinylated lipids and/or any other modification known to skilled in the art.

According to the invention, lipids and glycosphingolipids mixed in step i), may be in a particular concentration in moles percentage.

The concentration of lipids may be from 90% to 99.9 in mol %, for example from 95 to 99.9 in mol %.

The concentration of glycosphingolipids may be from 0.1% to 10 in mol %, for example from 0.1 to 5 in mol %.

According to the invention, the mixing step may be carried out in any adapted receptacle known to one skilled in the art. It may be for example a glass tube.

According to the invention, the drying step ii) may be carried out with any method and process known to one skilled in the art. For example it may be carried out by evaporation of solvents under nitrogen or argon atmosphere. The drying step may be carried out, for example in any adapted receptacle, for example in a glass tube.

According to the invention, the drying step ii) may further comprise, for example after evaporation of solvents, a removal step ii′). The removal step may be carried out using any method known to one skilled in the art adapted to remove solvent. It may be for example a Vacuum removal, for example with application of vacuum for 2 hours.

The removal step ii′) advantageously allows to remove, if necessary, any remaining solvents after the drying step ii).

Advantageously, the drying step allows the formation of a homogenous dried lipid film or a homogenous dried mixture on the wall of the receptacle, for example the glass tube.

One skilled in the art taking into consideration his technical knowledge would adapt the drying step, for example, in light of the lipids and/or glycosphingolipids used.

According to the invention, the dried mixture may be resuspended in step iii) in any adapted buffer. It may be for example suspended in buffer selected from the group comprising Phosphate Buffer Saline (PBS), Tris Buffer, Hepes Buffer, sucrose buffer, advantageously sucrose buffer.

According to the invention, the buffer may be warmed before using in step iii) at a temperature above the melting temperature of lipids used in the method.

One skilled in the art, taking into consideration his technical knowledge knows the melting temperature of lipids and would adapt this temperature in accordance with the used lipids. For example, when the lipid is DOPC, the temperature may be 65° C.

According to the invention, the dried mixture or dried lipids may be suspended at step iii) in a buffer at a concentration from 0.9 to 1.1 mg/mL, for example at a concentration of 1 mg/mL.

Advantageously, the dried mixture may be resuspended in step iii) in any adapted buffer in which magnetic particles have been previously added.

According to the invention, magnetic particles may be in any form suitable to the implementation of step iii), for example in the form of a ball, puck or asymmetrical geometric shape.

According to the invention, the size of the magnetic particles may be any size adapted to the implementation of step iii). For example, the magnetic particle may have a size of 10 nm to 100 μm or 0.1 to 10 μm.

Advantageously, when the buffer comprises magnetic particles it allows to form unilamellar vesicles or liposomes in which magnetic particles are incorporated.

According to the invention, the process may further comprise before step iv) a mixing step iii′). According to the invention, the mixing step iii′) may be carried out with any mixing method known to one skilled in the art adapted to mix solutions comprising lipids. It may be for example carried out using a vortex. The time of mixing step iii″′) may be from 1 to 8 minutes, for example from 3 to 6 minutes, for example of 5 minutes. One skilled in the art, taking into consideration his technical knowledge would adapt the time of step iii′) in light of the lipids used.

According to the invention, the freeze and thaw step iv) may comprises a freeze step iv′) followed by a thaw step iv″).

According to the invention, the freeze step iv′) may be carried with any method and process known to one skilled in the art adapted to freeze solutions comprising lipids. For example it may be carried out by a method using a cooling bath, for example a cooling bath formed with a solution comprising ethanol and dry ice also called ethanol/dry ice mix. For example the receptacle comprising the suspension solution obtained step iii) may be plunged into the cooling bath.

As used herein, one skilled in the art, taking into consideration his technical knowledge would adapt the cooling time in light of the lipids used.

According to the invention, the thaw step iv″) may be carried with any method and process known to one skilled in the art adapted to thaw solution comprising lipids.

For example it may be carried out by a method using a warm water bath by submerging the receptacle comprising the frozen solution obtained in step iv′) into the warm water bath. The temperature of the warm water bath may be at a temperature above the melting temperature of lipids used.

As used herein, one skilled in the art, taking into consideration his technical knowledge knows the melting temperature of lipids and would adapt this temperature in accordance with the used lipids. For example, when the lipid is DOPC, the temperature of the water bath may be 65° C.

One skilled in the art, taking into consideration his technical knowledge would adapt the thaw time in light of the lipids used.

According to the invention, freeze step iv′) and step iv″) may be repeated as a cycle 2 to 5 times, for example 2 to 4 times, for example 3 times.

According to the invention, the process may comprise after step iv″) a mixing step iv″′). According to the invention, the mixing step iv″′) may be carried out with any mixing method known to one skilled in the art adapted to mix solution comprising lipids. It may be for example carried out by using a vortex. The time of mixing step iv″′) may be from 0.5 to 2 minutes, for example of 1 minute. One skilled in the art, taking into consideration his technical knowledge would adapt the time of step iv″′) in light of the lipids used.

According to the invention, the extrusion step v) may be carried out with any process/device known form one skilled in the art adapted. It may be for example a purification step using an extruder, for example a commercially available extruder commercialized by Avanti Polar Lipids, Inc. (https://avantilipids.com/divisions/equipment/).

It may be for example an extrusion using supports with pores with a diameter from 30 to 1000 nm, for example support with pores with a diameter of 30, 50 100, 200, 400, 800, 1000 nm.

The support may be any support known to one skilled in the art adapted to filter solution comprising lipids. It may be for example a polycarbonate membrane (PC-membrane), for example a polycarbonate membrane commercialized by Avanti Polar Lipids, Inc.

According to the invention, the Extrusion step v) may be carried at a temperature above the melting temperature of lipids used.

According to the invention, extrusion step v) may be repeated be repeated 2 to 20 times, for example 5 to 18 times, for example 17 times.

Advantageously, the homogeneous preparation obtained after the extrusion step v) comprises liposomes with an homogeneous size.

Advantageously, the obtained homogeneous preparation may be stored and conserved before using it. For example, the obtained homogeneous preparation may be stored and conserved at 4° C.

According to the invention, the support comprising glycosphingolipids may be advantageously unilamellar vesicles or liposomes in which magnetic particles are incorporated.

Advantageously, the density of glycosphingolipids present on the surface of the unilamellar vesicles or liposomes is proportional to glycosphingolipids' concentration in the mix of lipids and glycosphingolipids.

According to the invention, step b) of incubation of the virus with the support comprising glycosphingolipids on its surface to let the virus bind to the glycosphingolipids can be carried out at a specific temperature; for example, this step can be carried out from 0 to 37° C. and preferably at least at 0° C.; for example, step b. can be carried out at a temperature of 0° C.

According to the invention, step b) of incubation can be carried out for a predetermined time; for example, this step can be carried out for at least 5 minutes and preferably at least 10 minutes; for example, step b. can be carried out for 30 to 90 minutes.

One skilled in the art taking into consideration his technical knowledge would adapt the incubation time and/or temperature of the incubation step for example in light of the support used.

According to the invention, step c) of washing may be carried out with any method known to one skilled in the art adapted for use with the invention. It may be for example comprise an elimination step of the incubation solution following with a washing step of the support with a rinsing solution.

The rinsing solution may be any solution known to a person skilled in the art adapted for use with the invention. It may be, for example a rinsing solution which does not alter the binding and the folding of proteins and glycosphingolipids. It may be for example a buffer medium, for example any buffer medium known to a person skilled in the art adapted, for example a commercially available buffer medium like phosphate buffered saline (PBS).

According to the invention, step d) of recovering the virus bounded to the glycosphingolipids may be carried out with any method known to one skilled in the art adapted for use with the invention. It may for example comprise a step of incubation of the bounded surface into a dissociating solution following with a washing step of the support with a rinsing solution. It may be, for example, a method of recovery or isolation as mentioned above.

According to the invention, when the support is unilamellar vesicles or liposomes the recovery step d) may be carried out by any method known to one skilled in the art. For example, such vesicles or liposomes could be recovered by centrifugation and elimination of the washing solution.

According to the invention, when the support is unilamellar vesicles or liposomes in which magnetic particles are incorporated the recovery step d) may be carried out by any method known to one skill in the art. For example, such vesicles or liposomes could be recovered by centrifugation and elimination of the washing solution, or further, and preferably according to the invention by using a system generating a magnetic or electric field capable of attracting the particles, particularly a magnet. For example, the vesicles or liposomes may sampled using a magnet which may be dipped into the sample. When the magnet which may be dipped into the sample. According to the invention, the system generating a magnetic or electric field capable of attracting the particles, particularly a magnet, may be protected, by any system, particularly by a removable coating or a cover, made of any material, for example, plastics, which does not interfere with magnetic or electric waves. More advantageously still, said cover is disposable after use. The unilamellar vesicles or liposomes in which magnetic particles are incorporated may be also recovered with a magnet which may pull down them into the solution.

According to the invention, the method of the invention for determining specificity for a molecule may be carried out on the library of viruses of the invention or a library of phages of the invention.

According to the invention, the method for determining the specificity of viruses to glycosphingolipids may comprise a preliminary step a′) comprising putting into contact, in a solution, viruses with a support which does not comprise glycosphingolipids on its surface and recovering said solution.

The solution, incubation conditions and support are as defined above.

Advantageously, the preliminary step a′) allows to eliminate the viruses which are nonspecific binders of glycosphingolipids.

According to the invention the method for determining the specificity of viruses to glycosphingolipids may comprise another preliminary step a″) comprising infecting cell with viruses to increase the number of viruses present in the solution.

The infection step may be carried out by any method known to one skilled in the art. It may be for example a method as previously mentioned.

According to the invention the preliminary steps a′) and a″) may be independently carried out before step a) of the method of the invention for determining the specificity of virus of the invention.

According to the present invention the preliminary steps a′) may be followed by step a″) before step a).

According to the present invention the preliminary steps a′) may be followed by step a″) may represent a cycle of steps which may be carried out 3 to 5 times before implementing step a).

Advantageously, when the process involves the repetition of step a′) followed by step a″), it advantageously allows the elimination of viruses which are nonspecific binders of glycosphingolipids and to increase the number of viruses to be tested.

According to the present invention, when in-vitro method for determining the specificity of a virus to glycosphingolipids is carried out with a library of viruses of the invention or a library of phages of the invention, it could advantageously allow to determine which viruses and/or phages of the library are able to bind a particular glycosphingolipids and which virus and/or phages of the library are not able to bind to said particular glycosphingolipid.

According to the present invention in-vitro method for determining the specificity of a virus to glycosphingolipids may comprise after step d) an analyzing step e) to determine the which phages were able to bind to the glycosphingolipids.

The analysis step may be any method known to one skilled in the art that could allow to determine the binding specificity of the peptides. It may be for example analysis using flow cytometry, immunofluorescence microscopy or pull down analysis. For example it may be a flow cytometry analysis wherein phages or virus are bound onto a glycosphingolipid presenting membrane, for example cells or liposomes as mentioned above, with a negative control, for example cells treated with a glycosphingolipid inhibitor, for example PPMP or liposomes with no glycosphingolipids or other glycosphingolipids to confirm the specificity. Appropriate fluorescent labelling may be performed, for example either direct conjugation of fluorophore onto peptides/phages, or with the use of fluorescently labelled antibodies, for example against said phages or virus, for example with an anti-M13 antibody. The cells/liposomes may be then passed through the FACS device, for example BD accuri, to collect for example 20 000 to 100 000 events. It may be for example analysis using Immunofluorescence microscopy comprising incubation of the phages of the invention or peptides of the invention on adherent cells which present or not glycosphingolipids on a coverslip, a step of fluorescent labelling and obtaining an imaged with epifluorescent microscope or spinning microscope for membrane binding. It may be also for example analysis using Pull down comprising incubation of phages of the invention or peptides of the invention with magnetic liposomes comprising or not comprising glycosphingolipids, recruiting the magnetic liposomes on a magnet and washing said liposomes with buffer, the obtained washed liposomes may be then boiled and loaded on a gel for Western Blot for migration and staining using antibodies allowing the quantification and analysis of the bound fractions on each liposome.

One skilled in the art taking into his technical knowledge knows various methods of analysis, for example flow cytometry, immunofluorescence microscopy or pull down analysis, and would select and/or adapt such methods to the peptides and/or fusion proteins and/or virus and/or phages of the invention.

The analysis step may also further comprises any method known to one skilled in the art to determine the peptide sequence. For example it may be a method comprising infecting bacteria with phages to obtain phage clones which may be used in sequencing methods. According to the invention, the sequencing method may be any sequencing method adapted known to one skilled in the art. For example, it may be a commercially available sequencing method, for example miniprep, and phagemid sequencing using classical primers such as T7 promoter Forward primers.

The invention also relates to a method to identify one or several filamentous phage(s) displaying an STxB-subunit or a variant thereof, according to the definitions provided herein, which bind, in particular specifically bind, to a particular glycosphingolipid or a variant thereof, or to a mix of several glycosphingolipids or variants thereof as a target, said method comprising:

a. Contacting, in particular under conditions enabling the binding with the target, a library of filamentous bacteriophages comprising a plurality of filamentous bacteriophages as defined herein, or a library of filamentous phages as defined herein, with one or several glycosphingolipid(s) or variants thereof displayed on a support, said support being for example cells expressing one or several glycosphingolipid(s) or variant thereof at their surface, or unilamellar vesicles or liposomes presenting one or several glycosphingolipid(s) or variant thereof, in particular unilamellar vesicles or liposomes in which magnetic particles are incorporated and presenting one or several glycosphingolipid(s) or variant thereof, and

b. Separating the filamentous bacteriophages that bind to the target from those that do not bind, for example through washing, and

c. Recovering the filamentous phage(s) bound to the target, and

d. Optionally, analyzing the filamentous phage(s) bound to the target and/or determining the sequence of at least a part of the nucleic acid content of the recovered filamentous phage(s) and/or the sequence of at least a part of the STxB-subunit or a variant thereof displayed by said recovered filamentous phage(s), especially the sequence of the STxB-subunit monomer or variant thereof, more particularly in the region responsible for the binding with the target.

The “conditions enabling the binding with the target” of step a) can be for example derived from the Experimental Section herein.

For illustration of “support being for example cells expressing one or several glycosphingolipid(s) or variant thereof at their surface, or unilamellar vesicles or liposomes presenting one or several glycosphingolipid(s) or variant thereof”, reference is for example made to Jones et al. (2016). Targeting membrane proteins for antibody discovery using phage display. Scientific Reports, 6(1), 26240 (Biopanning on cells) or Mirzabekov, Kontos, Farzan, Marasco, & Sodroski, 2000 (Magnetic liposomes). Also, in instant Experimental Section, last roundup of panning is performed on cells (CHO cells) to isolate Gb3 binders.

It will be understood that an advantage of magnetic liposomes is that this approach combines the properties of a minimal, controlled and modular system, resuming partly the biological context of the cell membrane. In the context of present invention, glycosphingolipids (GSLs) of choice are chemically synthetized or purified from natural sources, and then incorporated into large unilamellar vesicles. The formation of vesicles in the presence of magnetic particles allows for encapsulation of the latter. The generated liposomes are thus magnetic and can be recruited onto strong magnets.

By “target” in the context for said method, it is therefore meant a particular glycosphingolipid or a variant thereof, or a mix of several glycosphingolipids or variants, according to the definitions provided herein. According to particular examples, the target may be a particular glycosphingolipid or variant thereof, for example selected among: Gb3, Gb4, Forsmann like iGb4, fucosyl-GM1, GM1, GM2, GD2, Globo-H, NeuAc-GM3, NeuGc-GM3, GD1a, O-acetyl-GD3, O-acteyl-G D2, O-acetyl-GT3, GD3, but also mixtures thereof.

The “support” displaying one or several glycosphingolipid(s) or variant thereof, can be cells expressing one or several glycosphingolipid(s) or variant thereof at their surface, the method to identify one or several filamentous phage(s) displaying an STxB-subunit or a variant thereof being carried out in vitro. Cells may be recovered and/or isolated from a patient suffering or susceptible of suffering from one or several, in particular one neoplasic condition, selected among: ovarian cancer, breast carcinoma, colon cancer, gastric adenocarcinoma, Burkitt's lymphoma, colon carcinoma, melanoma, small cell lung cancer (SCLC), renal carcinoma, neuroblastoma, cervical carcinoma, glioblastoma, renal carcinoma, glioma, retinoblastoma, neuroectodermal cancer, non-small cell lung cancer (NSCLC), Wilms tumor, osteosarcoma, and t-All condition.

According to another embodiment, the support can be unilamellar vesicles or liposomes presenting one or several glycosphingolipid(s) or variant thereof.

According to another embodiment, the support can be unilamellar vesicles or liposomes in which magnetic particles are incorporated and presenting one or several glycosphingolipid(s) or variant thereof. A particular example describing preparation and retrieval of unilamellar vesicles or liposomes in which magnetic particles are incorporated and presenting glycosphingolipid(s) of interest is disclosed in the experimental section herein.

According to a particular embodiment, the method for identifying one or several filamentous phage(s) displaying an STxB-subunit or a variant thereof of the invention encompasses reiteration of steps a) to c) above several times, for example 2, 3, 4 or 5 times, each subsequent step a) using the filamentous phage(s) retrieved in step c) of the preceding iteration.

According to a particular embodiment, the method for identifying one or several filamentous phage(s) displaying an STxB-subunit or a variant thereof of the invention encompasses reiteration of steps a) to c) above several times, and is performed at least once, for example the first time, using as a target a mix of several glycosphingolipids or variants thereof, said mix being depleted in particular glycosphingolipid(s) or variants thereof constituting a desired target against which the filamentous phage(s) will be screened in a subsequent iteration.

It will be understood that such a succession of steps conveniently enables to remove from the library of filamentous bacteriophages, those which may diminish the efficiency of the identification method, and/or remove unspecific binders to the desired target.

The invention also relates to a method of identifying an STxB-subunit or a variant thereof according to the definitions provided herein, which bind, in particular specifically bind, to a particular glycosphingolipid or a variant thereof, or to a mix of several glycosphingolipids or variants thereof as a target, said method comprising analyzing the filamentous phage(s) recovered from step c) described above, at any stage of reiteration, if relevant.

Analyzing the recovered filamentous phage(s) can be performed using conventional and well known methods, such as flow cytometry, recruitment (pull down) on magnetic liposomes, ELISA on GSL coated plates, or immunofluorescence microscopy. Another relevant method encompasses sequencing, in, particular next generation sequencing. A binder should be enriched during the selection, meaning that in the final pool of bacteria, the variants that are found independently in different clones have great chance to be binders. This can be then confirmed by FACS and other techniques described herein and well known to the skilled in the art.

To identify an STxB-subunit or a variant thereof according to the definitions provided herein, which bind, in particular specifically bind, to a particular glycosphingolipid or a variant thereof, determination of the sequence of at least a part of the nucleic acid content of the recovered filamentous phage(s) can conveniently be achieved using well-known methods, especially sequencing methods. Any suitable sequencing method may be used. The skilled person is well aware of different sequencing that may be used.

Identification of the STxB-subunit or a variant thereof can also be performed through sequencing of the protein or peptidic sequence of the at least a part of the STxB-subunit or a variant thereof displayed by the recovered filamentous phage(s), or by predicting the amino acid sequence from the sequenced nucleotidic sequence of a region of interest within the genome of the recovered filamentous phage(s).

The skilled person can readily determine the region to be sequenced, or identify such a region of interest, which may be within the sequence of an STxB-subunit monomer or variant thereof as defined herein, more particularly in the region responsible for the binding with the target, which is readily discussed in the literature, and in the present description.

According to another aspect, the invention also relates to a filamentous bacteriophage displaying an STxB-subunit or a variant thereof at its surface of the invention, according to any one of the embodiments disclosed herein, or a composition comprising the same, for use as a medicament.

Filamentous bacteriophages of the invention may be used in the treatment of a patient suffering from one or several, in particular one neoplasic condition, selected among: ovarian cancer, breast carcinoma, colon cancer, gastric adenocarcinoma, Burkitt's lymphoma, colon carcinoma, melanoma, small cell lung cancer (SCLC), renal carcinoma, neuroblastoma, cervical carcinoma, glioblastoma, renal carcinoma, glioma, retinoblastoma, neuroectodermal cancer, non-small cell lung cancer (NSCLC), Wilms tumor, osteosarcoma, and t-All condition, or for vaccinating subjects in need thereof.

According to another aspect of the present disclosure, filamentous phages of the invention can also be used as detection tools, in particular in detection imaging methods, especially to target glycosphingolipid(s) as defined herein, in particular when such phages display binding or specific binding activity against glycosphingolipid(s) as defined herein. According to a particular embodiment, detection methods are carried out in vitro on a medium, or on a biological sample removed from an animal that may be a human, said medium or biological sample containing or being susceptible to contain glycosphingolipid(s) of interest to be detected. The sample removed from an animal may a biological sample recovered from a patient diagnosed or susceptible to have a pathological condition such as cancer, or a condition associated with glycosphingolipid(s) dysregulation.

To this end, filamentous phages of the invention can conveniently bear a detectable moiety, such as a label or a tag, including a detectable moiety associated, grafted, or coupled, including covalently bound, to the STxB-subunit or a variant thereof displayed at the surface, to the STxB-subunit or fusion protein. Suitable methods for achieving such a grafting are readily available to the skilled person.

According to a particular embodiment, a filamentous phage of the invention bearing a detectable moiety has unchanged functional properties, especially in terms of the glycosphingolipid's targeting or targeting specificity, with respect to a corresponding filamentous phage not bearing such a detectable moiety. The skilled person can readily assess whether functional properties remain unchanged, by comparison, using well known methods.

Appropriate tags/labels may be selected among the groups comprising biotin, fluorescent dyes for example rhodopsine, alexa-Fluor, nanogold coated ligands, carbon-black coated ligands, mangradex, fluorescent ligand such as fluorochromes, or radioactive molecules, for example comprising radioactive atoms for scintigraphic studies such as ¹²³I, ¹²⁴I, ¹¹¹In, ¹⁸⁶Re, ¹⁸⁸Re.

The invention therefore also relates to filamentous phages as defined herein, which are labelled, for use as a probe for in vivo detection of glycosphingolipid(s) of interest in human tissues or cells.

The invention therefore also relates to the use of filamentous phages as defined herein or a composition comprising the same, as a probe for staining glycosphingolipid(s) of interest, in vivo or in vitro, in particular in biological samples potentially containing the target of interest.

The present invention also refers to a chimeric protein comprising a peptide of the invention and a compound fused at one of its ends.

According to the invention the compound may be fused directly to the C-terminus or N-terminus of the peptide of the invention or with a linker. In the present, the linker may be at the C-terminus or N-terminus of the peptide. When the linker is at the N-terminus it may have the following formula: pep-Z(n)-Cys wherein pep is the peptide of the invention, Z is an amino-acid devoided of sulfydryl group, n being 0, 1 or an amino-acid sequence, and Cys is Cysteine amino acid.

In the present invention, the compound fused at its end may be a chemical or a biological compound. The compound may be drugs, for example haptenes, psoralenes, or any compounds provided that they have a chemical group linkable with the —SH group of the Cysteine moiety of pep-Z(n)-Cys.

The compound might be linked either directly or after activation with compounds such as bromoacetate, or any other method known by a skilled person, provided that the result of the reaction is a chemical entity having the following formula: pep-Cys-M, M being all the above mentioned compounds.

The coupling approaches for covalent binding of a peptidic or a polypeptidic moiety can be any method or processes known to one skilled in the art and/or described or carried out by one skilled in the art. For example, a first method that can be embodied is the use of SPDP hetero-bi-functional cross-linker described in Carlsson et al. 1978. Protein thiolation and reversible protein-protein conjugation. N-Succinimidyl 3-(2-pyridyldithio)propionate, a new heterobifunctional reagent. Biochem. J. 173:723-737. Another example of a method for covalently coupling the peptides of the invention or fusion protein of the invention with another peptide of interest is to produce bromoacetyl or maleimide functions on the latter as described by P. Schelte et al “Differential Reactivity of Maleimide and Bromoacetyl functions with Thiais: Application to the Preparation of Liposomal Diepitope Constructs”. Eur. J. Immunol. (1999) 29:2297-2308. Another example of a method for coupling a molecule to the peptide of the invention or fusion protein of the invention is to use MBS (m-Maleimido-benzoyl-N-hydroxysuccinimide ester). This coupling would allow the transport and processing of large compound such antigenic proteins through MHC class I and/or MHC class II pathways. Click chemistry may also be used for coupling purposes, as discussed above. The skilled person can readily adapt protocols of the art to this end.

According to the invention, the chimeric protein according to the present invention may allow to target compounds to particular tissue or cells expressing glycosphingolipids. For example, the chimeric protein according to the present invention may allow to target to an antigen presenting cells expressing a glycosphingolipid a compound which may be constituted by or may comprise a polypeptidic structure, such an antigen or epitopes of the chimeric protein, glycopeptides or glycoproteins, lipopeptides or lipoproteins.

According to the invention, the compound may be a polypeptide capable of binding with polynucleotide structures such as DNA, RNA or siRNA molecules. Such compounds might be vectors or plasmids comprising a sequence of interest to be expressed in a target cell. It may be also any siRNA molecule which may cause gene silencing through repression of transcription. In the present invention, a target cell is a eukaryotic cell bearing glycosphingolipids on its membrane. Thus, the chimeric protein of the present invention may also be a carrier for introducing a nucleotide sequence in a target cell either for gene therapy or for obtaining recombinant cells expressing heterologous proteins. The chimeric protein of the present invention may also be a carrier for introducing siRNA molecules in a target cell either for gene therapy or for silencing gene, for example involved in anarchic multiplication of cells.

According to the invention, the peptides of the present invention may be also operably linked directly through a covalent binding or indirectly through a linker to a cytotoxic drug.

Advantageously, the peptide of the invention would allow to target said cytotoxic drug to cells expressing glycosphingolipids. Advantageously, the peptide of the invention would allow to target said a cytotoxic drug to tumour cells expressing glycosphingolipids.

The term “indirect binding” means that the peptide of the invention or fusion peptide of the invention may be covalently linked through the sulfhydryl moiety of the C-terminal Cysteine to a linker, said linker being operably linked to a drug or a pro-drug to be internalized into glycosphingolipids bearing cells.

This linkage might be through covalent bonding or non-covalent bonding, provided that the activity of the peptide of the invention or of the fusion protein of the invention and the activity of the molecule are not modified.

The peptides of the invention offer new ways of recognizing target molecules that have been implicated in disease. In particular, the peptides of the invention allow recognition of glycosphingolipids which have been shown to be present on cancer cells. Therefore the present invention is susceptible to provide exciting new ways of detecting and treating certain cancers.

Advantageously, the peptides of the invention allow to target compounds, for example drugs to cells and/or tissue expressing glycosphingolipids and thus would allow improved treatment efficiency by increasing the concentration of the drug on the pathological site. In addition, the peptides of the invention would also allow to improve the medication compliance by reducing the potential side effect due to the drugs, in particular cytotoxic drugs on “heathy” tissues and/or cells.

Accordingly, the present invention also pertains to a chimeric protein according to the invention for use as a medicament.

According to the invention, the medicament may be useful for the treatment of diseases in which glycosphingolipids are misregulated. Examples of diseases in which in which glycosphingolipids are misregulated are mentioned above.

In the present, the medicament may be in any form which can be administered to a human or animal. The administration may be carried out directly, i.e. with the medicament in a pure or substantially pure form, with a pharmaceutically acceptable carrier and/or carrier.

According to the present invention, the medicament may be in the form of a powder, for example for injectable solution, or for oral administration to be swallowed in the form of capsules.

According to the present invention, the medicament may be a medicament for oral administration. For example, when the medicament is a medicament for oral administration, it may, for example, be in the form of a capsule.

According to the present invention, the medicament may be for parenteral administration, for example intravenous administration.

The objects and aspects listed hereafter are part of the present invention and disclosure:

Item 1. A peptide of amino acid sequence of

XaPDCVTGKVEYTKYNXbXcXdTFXeVKVGDKXfXgXhXiXjXkXlXmLQSLLLSA QITGMTVTIKXnXoXpCHNXqGXrXsXtEVIFR

wherein
Xa is T, A or S,
Xb, Xc, Xd, Xf, Xm are independently D, E or N,
Xe, Xi, Xn, Xp, Xt are independently T, A or S,
Xg is L, I or V,
Xh is F, Y, W or A,
Xj, is N, E or S,
Xk is R, K or E
Xl is W, F, Y or A,
Xo is N, E, D or S,
Xq is G A or S,
Xr is G, A, S or T
Xs is F, L or Y,

provided that when Xa is T, Xb, Xc, Xd are not D, Xe is not T, Xf is not E, Xg is not L, Xh is not F, Xi is not T, Xj is not N, Xk is not R, Xl is not W, Xm is not N, Xn is not T, Xo is not N, Xp is not A, Xq is not G, Xr is not G, Xs is not F and Xt is not S.

Item 2. A fusion protein comprising a peptide of Item 1 or a peptide of SEQ ID NO: 1, said peptide according to Item 1 or said peptide of SEQ ID NO: 1 is fused to a coat protein of a virus.
Item 3. A fusion protein of Item 2 wherein the coat protein of a virus is a pIII protein phage coat protein.
Item 4. A nucleic acid coding for the peptide according to Item 1.
Item 5. A nucleic acid coding for a fusion protein of Item 2 or 3.
Item 6. A nucleic acid according Item 5, wherein the nucleic acid comprises at the end of the sequence coding for the peptide of Item 1 or for the peptide of SEQ ID NO: 1 a stop codon.
Item 7. An expression system comprising a nucleic acid coding for the peptide of amino acid sequence XaPDCVTGKVEYTKYNXbXcXdTFXeVKVGDKXfXgXhXiXjXkXlXmLQSLLLSAQITGMT VTIKXnXoXpCHNXqGXrXsXtEVIFR

wherein
Xa is T, A or S,
Xb, Xc, Xd, Xf, Xm are independently D, E or N,
Xe, Xi, Xn, Xp, Xt are independently T, A or S,
Xg is L, I or V,
Xh is F, Y, W or A,
Xj, is N, E or S,
Xk is R, K or E
Xl is W, F, Y or A,
Xo is N, E, D or S,
Xq is G A or S,
Xr is G, A, S or T
Xs is F, L or Y.

wherein the expression system is at least one of a plasmid, a phagemid or an expression vector.

Item 8. An expression system comprising a nucleic acid according to Item 4 wherein the expression system is at least one of a plasmid, a phagemid or an expression vector.
Item 9. A host comprising a peptide according to Item 1 or a fusion protein according to Items 2 or 3 and/or an expression system according to Items 7 or 8.
Item 10. A host according to Item 9 wherein the host is a eukaryote cell, a prokaryote cell.
Item 11. A virus comprising a peptide of Item 1 and/or a fusion protein of Items 2 and/or 3 on the surface thereof.
Item 12. A virus displaying a peptide of Item 1 and/or a fusion protein of Items 2 and/or 3 on the surface thereof.
Item 13. A library of viruses, comprising a plurality of virus of Item 12 displaying a plurality of different peptides of Item 1 and/or fusion proteins of Items 2 and/or 3 on the surface thereof.
Item 14. A library of viruses according to Item 13, wherein the viruses are phages.
Item 15. A method for producing a peptide according to Item 1 or a fusion protein according to Items 2 or 3 by genetic recombination using a nucleic acid according to any of Items 4 to 6 or an expression system according to Items 7 or 8.
Item 16. A method for producing a library of phages comprising:

a) Infecting bacteria with phages comprising phagemid expressing a nucleic acid of Item 6 or transforming bacteria with phagemid expressing a nucleic acid of Item 6,

b) Optionally infecting the infected bacteria with an amount of helper phage encoding a phage coat protein sufficient to produce recombinant phagemid particles which display the fusion protein on the surface of the particles

c) culturing the transformed infected bacteria under conditions suitable for forming a library of phage displaying expressing peptide of Item 1 or fusion protein of Item 2

Item 17. In vitro use of a peptide of Item 1 and/or a fusion protein of Item 2 or a host of Item 9 or a virus of Item 11 or a library of viruses of Item 13 or 14 for detecting molecule and/or a cell in a sample.
Item 18. In vitro use according to Item 17 wherein the molecule is a glycosphingolipid.
Item 19. Method for determining the specificity of virus according to Item 14 to glycosphingolipids.
Item 20. Method for determining the specificity of virus according to Item 19 to glycosphingolipids comprising the following steps:

a) Putting into contact a virus according to Item 14 and a support comprising glycosphingolipids on its surface,

b) Incubating the virus and the support comprising glycosphingolipids present on its surface to let the virus bind to the glycosphingolipids,

c) Washing the incubated surface to eliminate the non-bounded virus, and

d) Recovering virus bounded to the glycosphingolipids.

Item 21. A chimeric protein comprising a peptide as defined in Item 7 and a compound fused at one of its end.
Item 22. A chimeric protein according to Item 21 for use as a medicament.
Item 23. A chimeric protein according to Item 22 for use as a medicament for the treatment of diseases in which glycosphingolipids are misregulated.
Item 24. Use of a peptide of Item 1 and/or a fusion protein of Item 2 or a host of Item 9 or a virus of Item 11 or a library of viruses of Item 13 or 14 in an in vitro or in vivo diagnostic method.
Item 25. Use of a peptide of Item 1 and/or a fusion protein of Item 2 or a host of Item 9 or a virus of Item 11 or a library of viruses of Item 13 or 14 in an in vitro or in vivo diagnostic method.
Item 26. Use of a peptide of Item 1 and/or a fusion protein of Item 2 or a host of Item 9 or a virus of Item 11 or a library of viruses of Item 13 or 14 in an in vitro method for detecting a disease in which glycosphingolipids are misregulated.

To assist the reader of the present application, the description has been separated into various paragraphs or sections and/or in various embodiments. These separations should not be considered as disconnecting the substance of a paragraph or section and/or of an embodiment from the substance of another paragraph or section and/or of another embodiment. To the contrary, the present application encompasses all the combinations of the various sections, paragraphs and sentences that can be contemplated. The present application encompasses all the combinations of the various embodiments that are herein described.

In the application, unless specified otherwise or unless a context dictates otherwise, all the terms have their ordinary meaning in the relevant field(s).

The term “comprising”, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

The term “essentially consisting of” is a partially open term, which does not exclude additional, unrecited element(s), step(s), or ingredient(s), as long as these additional element(s), step(s) or ingredient(s) do not materially affect the basic and novel properties of the invention.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In an attempt to help the reader of the present application, the description has been separated in various paragraphs or sections and/or in various embodiments. These separations should not be considered as disconnecting the substance of a paragraph or section and/or of an embodiment from the substance of another paragraph or section and/or of another embodiment. To the contrary, the present application encompasses all the combinations of the various sections, paragraphs and sentences that can be contemplated. The present application encompasses all the combinations of the various embodiments that are herein described.

Each of the relevant disclosures of all references cited herein is specifically incorporated by reference. The following examples are offered by way of illustration, and not by way of limitation.

Other examples and features of the invention will be apparent when reading the examples and the figures, which illustrate the experiments conducted by the inventors, in complement to the features and definitions given in the present description.

### BRIEF DESCRIPTION OF THE FIGURES

FIGS. 1A-1D are photographs of cells imaged by epifluorescence microscopy. On this figure Gb3+CHO and Gb3-CHO cells means CHO cells expressing (Gb3+) or not (Gb3-) the glycosphingolipid Gb3. FIGS. 1A and 1B represent the photographs of binding of Alexa_488 labeled B-subunit of Shiga toxin (STxB) on respectively Gb3+CHO and Gb3-CHO for 30 min at 4° C. FIGS. 1C and 1D represent the photographs of binding of phage displaying peptide of sequence SEQ ID NO: 1 (B-subunit of Shiga toxin (Φ_STxB)) on Gb3+CHO and Gb3-CHO cells for 45 min at 4° C. detected by immunofluorescence using the M13 antibody.

FIGS. 2A-2D are the flowcharts of binding of peptide of sequence SEQ ID NO: 1 (STxB) and Φ_STxB on Gb3+CHO and Gb3-CHO cells analyzed by flow cytometry. FIGS. 2 A and B represent the flowcharts of binding of Alexa_488 labeled STxB on respectively Gb3+CHO and Gb3-CHO for 30 min at 4° C. FIGS. 2 C and D represent the binding of Φ_STxB on Gb3+CHO and Gb3-CHO for 45 min at 4° C. detected by immunofluorescence using the M13 antibody.

FIGS. 3A and 3B are photographs illustrating retrograde trafficking of STxB to the Golgi apparatus in cells imaged by immunofluorescence. FIGS. 3 A and 3B are photographs of Alexa_488 labeled STxB incubated respectively on GB3+CHO and Gb3-CHO for 45° C. at 37° C.

FIGS. 4A and 4B are schematic representations of the expression vector and bacteriophage. FIG. 4A represents pHEN2 expressing vector comprising B-subunit of Shiga toxin sequence (STxB) fused with protein pIII sequence, FIG. 4B represent M13 Bacteriophage presenting STxB.

FIG. 5 is a photograph of a nitrocellulose membrane obtained after immunoblotting. On this photography, the black line corresponds to a fusion protein of the peptide of sequence SEQ ID NO: 1 and pIII protein phage coat protein (STxB_PIII) and a fusion protein of a mutated peptide B-subunit of Shiga toxin sequence and pIII protein phage coat protein (STxB_mut_PIII fusion protein) expressed by TG1 bacteria and isolated into the supernatant of culture medium.

FIGS. 6A-6D are photographs of cells imaged by epifluorescence microscopy. FIG. 6 A is a photograph showing the binding of Alexa_488 labeled STxB on C2TA cells without 2-[4-(3,4-dimethoxyphenyl)-3-methyl-1H-pyrazol-5-yl]-5-[(2-methylprop-2-en-1-yl)oxy] phenol (PPMP) treatment. FIG. 6 B is a photography showing the binding of Alexa_488 labeled STxB C2TA cells after PPMP treatment. The loss of STxB binding on C2TA treated with PPMP confirmed the inhibition of glycosphingolipids synthesis. FIG. 6 C is a photograph showing the binding of Alexa_488 labeled Φ_STxB on C2TA cells without PPMP treatment. FIG. 6 D is a photography showing the binding of Alexa_488 labeled Φ_STxB on C2TA cells after PPMP treatment. On these images light spots correspond to Alexa_488 labeled STxB. After inhibition of glycosphingolipids synthesis (PPMP treated C2TA), the binding of O_STxB is lost confirming the binding on a glycosphingolipid.

FIG. 7 is a photograph of a polyacrylamide gel stained with Labsafe Gel Blue loaded with culture supernatent of TG1 bacteria expressing STxB_mut_PIII fusion protein, results obtained by mass spectrometry analysis and the amino acid sequence of STxB_mut_PIII fusion protein. 13 matching peptides covering both mutated amino acids of STxB mutant and PIII were detected. (Sequence on FIG. 7 corresponds to SEQ ID NO: 33 herein)

FIGS. 8A and 8B are photographs of cells imaged by epifluorescence microscopy. FIG. 8 A is a photography showing the binding Φ_STxB on C2TA cells imaged by epifluorescence microscopy. FIG. 8 B is a photograph showing the binding Φ_STxB_mut on C2TA cells imaged by epifluorescence microscopy. Binding of the O_STxB_mut is lost which confirm that displaying of STxB on the M13 bacteriophage specifically drives its binding on Gb3+ cells.

FIGS. 9A-9C are flowcharts of Binding of STxB_488, Φ_STxB and Φ_STxB_mut on C2TA cells analyzed by flow cytometry. FIG. 9 A represent the flowcharts of binding of Alexa_488 labeled STxB. FIG. 9 B represent the flowcharts of binding of Φ_STxB detected by immunofluorescence using the M13 antibody. Binding of the O_STxB on C2TA cells confirmed is ability to recognize Gb3. FIG. 9 C the flowcharts of binding of Φ_STxB_mut detected by immunofluorescence using the M13 antibody. Loss of binding when glycosphingolipid synthesis is inhibited again confirm that displaying of STxB on the M13 bacteriophage specifically drives its binding on Gb3+ cells.

FIG. 10 is a schematic representation of Phage display selection of peptide of the invention that bind specifically glycosphingolipids. The library of phage display of the invention (1) is first depleted on cells which does not expressed the desire target (2) in order to remove unspecific binders (3). Unbound phages are then recruited on cells expressing the target (4). After washing (5), remaining phages are eluted and amplified (6) and use for another round of selection. After 3 to 5 cycles, phages are tested for specific binding on the desire target (7).

FIGS. 11A and 11B illustrate conformation of STxB protein when displayed on the M13 bacteriophage.

5A. Five STxB monomers, each of them fused with one pIII protein of the phage, were able to pentamerize. Only one pentamer of STxB could be then displayed on a phage particle. 5B. One STxB monomer in fusion with the pIII protein was able to pentamerize with 4 others free STxB monomer in the periplasm of the bacteria during the assembly of the phage particles. One to five STxB pentamer could then be displayed on a phage particle.

FIGS. 12A-12D illustrate binding of OSTxB with and without amber stop codon (OSTxB & O_STxB_NAmb) and with standard helper phage (HP), or hyperphage (HY) on C2TA cells imaged by epifluorescence microscopy. A. O_STxB_NAmb_HP B. O_STxB_NAmb_HY C. O_STxB_HP D. O_STxB_HY. The absence of the amber stop codon (Namb), meaning 100% of fusion of STxB_pIII resulted in a phage preparation, both with the use of hyper and helper phages, which was no longer able to bind Gb3 positive cells.

FIGS. 13A and 13B illustrate expression of STxBPIII fusion, using combination of amber stop codon and helper/hyper phages, into the supernatant of TG1 bacteria is confirmed by immunoblotting using the antibody against the pIII protein.

FIG. 14 illustrates magnetic unilamellar vesicles (MUVs) for the specific recruitment of Gb3 binders. Legend (1) Magnetic particles, (2) STxB, (3) Gb3, (4) lipid bilayer (DOPC), (5) magnet.

FIGS. 15A and 15B illustrate specific recruitment of STxB (A.) and phages displaying STxB (B.) on Gb3 positive MUVs and not on control MUVs confirmed by immunoblotting using anti_STxB antibody (A.) and anti_pIII antibody (B.)

FIGS. 16.1 and 16.2 illustrate specific recruitment of STxB and Φ_STxB onto Gb3 containing magnetic liposomes. 1. Electron microscopy characterization of magnetic liposomes. 2. Specific recruitment of STxB and Ph_STxB on GB3 containing liposomes. Ph_STxB_mut is not recruited on neither Gb3 nor DOPC liposomes and used here as a control. 3. Flow cytometry analysis of STxB and Φ_STxB recruitment on liposomes. A shift in fluorescence is observed for STxB and STxB when recruited on Gb3 containing liposomes. No significant recruitment could be observed on Gb3-negative liposomes. Furthermore, no significant recruitment of Φ_STxB_mut could be observed, neither on Gb3-negative nor on Gb3-positive liposomes. Likewise, the B-subunit of cholera toxin (CtxB) was not recruited on liposomes either, demonstrating that recruitment of STxB and Φ_STxB occurred through their binding to Gb3.

FIGS. 17.A.1.1 through FIG. 17.b illustrate characterization of 13 selected Gb3 binders. A) FACS analysis of phage binding on Hela (Red) and HeLa treated with PPMP (GSL inhibition-Blue). B) Sequence alignment of enriched sequences LB01, LB02 and LB03.

FIG. 18 illustrates specific binding of selected phages on Gb3 positive cells. Each clone was tested for binding on Gb3+CHO and Gb3-CHO cells. Each of them showed significant binding on Gb3+CHO, and not on Gb3-CHO.

FIG. 19 illustrates specific binding of selected STxB variants expressed in solution on Gb3 positive cells. Each clone was tested for binding on Gb3+CHO and Gb3-CHO cells. Each of them showed significant binding on Gb3+CHO, and not on Gb3-CHO.

### SEQUENCES

The amino acid sequence of B-subunit of Shiga toxin used is TPDCVTGKVEYTKYNDDDTFTVKVGDKELFTNRWNLQSLLLSAQITGMTVTIKTNACH NGGGFSEVIFR (SEQ ID NO: 1)

The consensus sequence defined in SEQ ID NO: 2 is: XaPDCVTGKVEYTKYNXbXcXdTFXeVKVGDKXfXgXhXiXjXkXIXmLQSLLLSAQITGMT VTIKXnXoXpCHNXqGXrXsXtEVIFR wherein Xa to Xt are as defined in instant description herein.

The amino acid sequence of STxB polypeptide of clones A3-D10-H3 is:

(SEQ ID NO: 3)
SPDCVTGKVEYTKYNNDDTFTVKVGDKELWTEKWNLQSLLLSAQ
ITGMTVTIKSNACHNGGSFAEVIFR

Nucleic acid sequence encoding SEQ ID NO: 3 is:

(SEQ ID NO: 4)
TCTCCTGATTGTGTAACTGGAAAGGTGGAGTATACAAAATATAATAAC
GACGACACCTTTACTGTTAAAGTGGGTGATAAAGAACTGTGGACTGAA
AAATGGAACCTTCAGTCTCTTCTTCTCAGTGCGCAAATTACGGGGATG
ACTGTAACCATTAAATCTAACGCATGTCATAATGGTGGGTCTTTTGCA
GAAGTTATTTTTCGT

The amino acid sequence of STxB polypeptide of clones B12-C03-D12-G05-G11-H11 is:

(SEQ ID NO: 5)
SPDCVTGKVEYTKYNNDDTFTVKVGDKELWTEKWNLQSLLLSAQITGM
TVTIKSNACHNGGSFAEVIFR

Nucleic acid sequence encoding SEQ ID NO: 5 is:

(SEQ ID NO: 6)
GCACCTGATTGTGTAACTGGAAAGGTGGAGTATACAAAATATAATAAC
GACGACACCTTTTCTGTTAAAGTGGGTGATAAAGAACTGTGGACTGAA
AAATGGAACCTTCAGTCTCTTCTTCTCAGTGCGCAAATTACGGGGATG
ACTGTAACCATTAAAACTAACGCATGTCATAATGGTGGGGCACTGTCT
GAAGTTATTTTTCGT

The amino acid sequence of STxB polypeptide of clones A06-C06 is:

(SEQ ID NO: 7)
SPDCVTGKVEYTKYNNDDTFSVKVGDKEIYTSKWNLQSLLLSAQ
ITGMTVTIKSNTCHNGGAFSEVIFR

Nucleic acid sequence encoding SEQ ID NO: 7 is:

(SEQ ID NO: 8)
TCTCCTGATTGTGTAACTGGAAAGGTGGAGTATACAAAATATAATAAC
GACGACACCTTTTCTGTTAAAGTGGGTGATAAAGAAATCTACACTTCT
AAATGGAACCTTCAGTCTCTTCTTCTCAGTGCGCAAATTACGGGGATG
ACTGTAACCATTAAATCTAACACTTGTCATAATGGTGGGGCATTTTCT
GAAGTTATTTTTCGT

The amino acid sequence of STxB polypeptide of clone B02 is:

(SEQ ID NO: 9)
SPDCVTGKVEYTKYNDEDTFSVKVGDKEVWTNRCKLQSLLLSAQ
ITGMTVTIKTSSCHNAGGLTEVIFR

Nucleic acid sequence encoding SEQ ID NO: 9 is:

(SEQ ID NO: 10)
TCTCCTGATTGTGTAACTGGAAAGGTGGAGTATACAAAATATAATGAC
GAAGACACCTTTTCTGTTAAAGTGGGTGATAAAGAAGTGTGGACTAAC
CGTTGCAAACTTCAGTCTCTTCTTCTCAGTGCGCAAATTACGGGGATG
ACTGTAACCATTAAAACTTCTTCTTGTCATAATGCAGGGGGTTTGACT
GAAGTTATTTTTCGT

The amino acid sequence of STxB polypeptide of clone B05 is:

(SEQ ID NO: 11)
APDCVTGKVEYTKYNDDNTFSVKVGDKELYTNRWNLQSLLLSAQITGM
TVTIKTNSCHNGGGFAEVIFR

Nucleic acid sequence encoding SEQ ID NO: 11 is:

(SEQ ID NO: 12)
GCACCTGATTGTGTAACTGGAAAGGTGGAGTATACAAAATATAATGAC
GACAACACCTTTTCTGTTAAAGTGGGTGATAAAGAACTGTACACTAAC
CGTTGGAACCTTCAGTCTCTTCTTCTCAGTGCGCAAATTACGGGGATG
ACTGTAACCATTAAAACTAACTCTTGTCATAATGGTGGGGGTTTTGCA
GAAGTTATTTTTCGT

SEQ ID NO: 13 (MKKTLLIAASLSFFSASALA) corresponds to a signal peptide.

SEQ ID NO: 14 is the concatenation of SEQ ID NO: 13 and SEQ ID NO: 1:

MKKTLLIAASLSFFSASALATPDCVTGKVEYTKYNDDDTFTVKVGDKE
LFTNRWNLQSLLLSAQITGMTVTIKTNACHNGGGFSEVIFR

Nucleic acid sequence encoding the signal peptide of SEQ ID NO: 13:

(SEQ ID NO: 15)
ATGAAAAAAACATTATTAATAGCTGCATCGCTTTCATTTTTTTCAGCA
AGTGCGCTGGCG

Exemplary M13 pIII sequence:

(SEQ ID NO: 16)
TVESCLAKPHTENSFTNVWKDDKTLDRYANYEGCLWNATGVVVC
TGDETQCYGTWVPIGLAIPENEGGGSEGGGSEGGGSEGGGTKPP
EYGDTPIPGYTYINPLDGTYPPGTEQNPANPNPSLEESQPLNTF
MFQNNRFRNRQGALTVYTGTVTQGTDPVKTYYQYTPVSSKAMYD
AYWNGKFRDCAFHSGFNEDPFVCEYQGQSSDLPQPPVNAGGGSG
GGSGGGSEGGGSEGGGSEGGGSEGGGSGGGSGSGDFDYEKMANA
NKGAMTENADENALQSDAKGKLDSVATDYGAANGDA

Example of STxB—PIII fusion protein:

(SEQ ID NO: 17)
MATPDCVTGKVEYTKYNDDDTFTVKVGDKELFTNRWNLQSLLLSAQITGM
TVTIKTNACHNGGGFSEVIFRAAAHHHHHHGAAEQKLISEEDLNGAAEQK

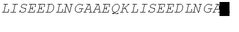

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Legend for SEQ ID NO: 17 is as follows:

Restriction Sites (NcoI) Hang Over
STxB

Histidine Tag

Myc Tag (3 Repeats)

Linkers
[symbol] position of amber stop codon, which is expressed as a Q in TG1 (amber-suppressor Host) bacteria: it allows for co-expression of STxB monomer and STxB_pIII fusion for pentameric assembly in the perisplasm of non amber-suppressor Host bacteria. In non amber-suppressor Host, it is expressed as a stop codon, in amber-suppressor Host, as Q.

PIII Fragment

The legend for SEQ ID NO: 17 applies similarly, in all correspondence, to all of SEQ ID NO: 20, 22, 24, 26, 28, respectively.

SEQ ID NO: 18 is the nucleic acid sequence encoding SEQ ID NO: 17:

atgGCGACGCCTGATTGTGTAACTGGAAAGGTGGAGTATACAAAATATAA
TGATGACGATACCTTTACGTTAAAGTGGGTGATAAAGAATTATTTACCAA
CAGATGGAATCTTCAGTCTCTTCTTCTCAGTGCGCAAATTACGGGGATGA
CTGTAACCATTAAAACTAATGCCTGTCATAATGGAGGGGGATTCAGCGAA
GTTATTTTTCGTGCggccGCACATCATCATCACCATCACGGGGCCGCgGA
ACAAAAACTCATCTCAGAAGAGGATCTGAATGGGGCCGCAgagcaaaagc
taatatctgaagaagatctcaacGGGGCCGCAgaacagaaacttatcagt

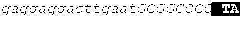

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

(Please note that to remove amber stop codon [symbol] , it can be replaced by codon CAG encoding a Q residue, thereby producing a fully fused protein). The legend for SEQ ID NO: 18 follows that of SEQ ID NO: 17 described above and applies similarly, in all correspondence, to all of SEQ ID NO: 21, 23, 25, 27, 29, respectively.

SEQ ID NO: 30 is the nucleic acid sequence encoding SEQ ID NO: 1:

ACGCCTGATTGTGTAACTGGAAAGGTGGAGTATACAAAATATAA
TGATGACGATACCTTTACAGTTAAAGTGGGTGATAAAGAATTAT
TTACCAACAGATGGAATCTTCAGTCTCTTCTTCTCAGTGCGCAA
ATTACGGGGATGACTGTAACCATTAAAACTAATGCCTGTCATAA
TGGAGGGGGATTCAGCGAAGTTATTTTTCGT

SEQ ID NO: 31 represent the nucleic acid sequence encoding STxB D18E, G62T mutant of SEQ ID NO: 32 (Bold nucleotides are mutated positions):

ACGCCTGATTGTGTAACTGGAAAGGTGGAGTATACAAAATATAA
TGATGAAGATACCTTTACAGTTAAAGTGGGTGATAAAGAATTAT
TTACCAACAGATGGAATCTTCAGTCTCTTCTTCTCAGTGCGCAA
ATTACGGGGATGACTGTAACCATTAAAACTAATGCCTGTCATAA
TGGAGGGACATTCAGCGAAGTTATTTTTCGT

SEQ ID NO: 32 is the STxB D18E, G62T mutant sequence (Bold residues are mutated positions):

TPDCVTGKVEYTKYNDEDTFTVKVGDKELFTNRWNLQSLLLSAQ
ITGMTVTIKTNACHNGGTFSEVIFR

SEQ ID NO: 33 corresponds to the STxB mutant of SEQ ID NO: 31 fused to pIII:

MATPDCVTGKVEYTKYNDEDTFTVKVGDKELFTNRWNLQSLLLSAQITGM
TVTIKTNACHNGGTFSEVIFRAAAHHHHHHGAAEQKLISEEDLNGAAEQK

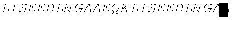

[symbol]

[symbol]

[symbol]

[symbol]

[symbol]

[symbol]

[symbol]

[symbol]

[symbol]

[symbol]

[symbol]

[symbol]

Legend for SEQ ID NO: 33 is as follows:

Restriction Sites (NcoI) Hang Over
D18E, G62T STxB mutant

Histidine Tag

Myc Tag (3 Repeats)

Linkers
[symbol] position of amber stop codon, which is expressed as a Q in TG1 (amber-suppressor Host) bacteria: it allows for co-expression of STxB monomer and STxB_pIII fusion for pentameric assembly in the perisplasm of non amber-suppressor Host bacteria. In non amber-suppressor Host, it is expressed as a stop codon, in amber-suppressor Host, as Q.

PIII Fragment

SEQ ID NO: 34 is nucleic acid sequence corresponding to SEQ ID NO: 33 (same legend applies):

ATGGCGACGCCTGATTGTGTAACTGGAAAGGTGGAGTATACAAAATATAA
TGATGAAGATACCTTTACAGTTAAAGTGGGTGATAAAGAATTATTTACCA
ACAGATGGAATCTTCAGTCTCTTCTTCTCAGTGCGCAAATTACGGGGATG
ACTGTAACCATTAAAACTAATGCCTGTCATAATGGAGGGACATTCAGCGA
AGTTATTTTTCGTGCggccGCACATCATCATCACCATCACGGGGCCGCgG
AACAAAAACTCATCTCAGAAGAGGATCTGAATGGGGCCGCAgagcaaaag
ctaatatctgaagaagatctcaacGGGGCCGCAgaacagaaacttatcag

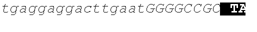

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

SEQ ID NO: 35 is a 4804 bp nucleic acid sequence as described in present description.

SEQ ID NO: 36 corresponds to SEQ ID NO: 1 with the first amino-acid residue being A:

APDCVTGKVEYTKYNDDDTFTVKVGDKELFTNRWNLQSLLLSAQ
ITGMTVTIKTNACHNGGGFSEVIFR

This sequence is incidentally described in “Functional analysis of the Shiga toxin and Shiga-like toxin type II variant binding subunits by using site-directed mutagenesis.” Jackson M. P., Wadolkowski E. A., Weinstein D. L., Holmes R. K., O'Brien A. D. J. Bacteriol. 172:653-658 (1990).

The consensus sequence defined in SEQ ID NO: 37 is: XaPDCVTGKVEYTKYNXbDDTFXeVKVGDKEXgXhTXjXkWNLQSLLLSAQITGMTVTIK XnNXpCHNGGXrXsXtEVIFR where Xa, Xb, Xe, Xg, Xh, Xj, Xk, Xn, Xp, Xr, Xs, Xt are as defined in instant description herein.

SEQ ID NO: 38 corresponds to so-called Scaffold section 51 of Table 1: PDCVTGKVEYTKYN.

SEQ ID NO: 39 corresponds to so-called Scaffold section S3 of Table 1: VKVGDK.

SEQ ID NO: 40 corresponds to so-called Scaffold section S4 of Table 1: LQSLLLSAQITGMTVTIK.

SEQ ID NO: 41 corresponds to so-called Scaffold section S7 of Table 1: EVIFR.

SEQ ID NO: 42 is wild-type pIII protein having 424 amino-acids residues:

MKKLLFAIPLVVPFYSHSAETVESCLAKPHTENSFTNVWKDDKT
LDRYANYEGCLWNATGVVVCTGDETQCYGTWVPIGLAIPENEGG
GSEGGGSEGGGSEGGGTKPPEYGDTPIPGYTYINPLDGTYPPGT
EQNPANPNPSLEESQPLNTFMFQNNRFRNRQGALTVYTGTVTQG
TDPVKTYYQYTPVSSKAMYDAYWNGKFRDCAFHSGFNEDPFVCE
YQGQSSDLPQPPVNAGGGSGGGSGGGSEGGGSEGGGSEGGGSEG
GGSGGGSGSGDFDYEKMANANKGAMTENADENALQSDAKGKLDS
VATDYGAAIDGFIGDVSGLANGNGATGDFAGSNSQMAQVGDGDN
SPLMNNFRQYLPSLPQSVECRPFVFSAGKPYEFSIDCDKINLFR
GVFAFLLYVATFMYVFSTFANILRNKES

Several mutant sequences are known in the art, which are not part of instant invention, only as far as isolated polypeptides are considered. For instance, Jackson M. P., Wadolkowski E. A., Weinstein D. L., Holmes R. K., O'Brien A. D. describe in “Functional analysis of the Shiga toxin and Shiga-like toxin type II variant binding subunits by using site-directed mutagenesis.” J. Bacteriol. 172:653-658 (1990), D16N SEQ ID NO: 43), D17N (SEQ ID NO: 44), D17E (SEQ ID NO: 45), D16N D17N (SEQ ID NO: 46), D18N (SEQ ID NO: 47) mutants.

(D16N):
SEQ ID NO: 43
TPDCVTGKVEYTKYNNDDTFTVKVGDKELFTNRWNLQSLLLSAQ
ITGMTVTIKTNACHNGGGFSEVIFR
(D17N):
SEQ ID NO: 44
TPDCVTGKVEYTKYNDNDTFTVKVGDKELFTNRWNLQSLLLSAQ
ITGMTVTIKTNACHNGGGFSEVIFR
(D17E):
SEQ ID NO: 45
TPDCVTGKVEYTKYNDEDTFTVKVGDKELFTNRWNLQSLLLSAQ
ITGMTVTIKTNACHNGGGFSEVIFR
(D16N D17N):
SEQ ID NO: 46
TPDCVTGKVEYTKYNNNDTFTVKVGDKELFTNRWNLQSLLLSAQ
ITGMTVTIKTNACHNGGGFSEVIFR
(D18N):
SEQ ID NO: 47
TPDCVTGKVEYTKYNDDNTFTVKVGDKELFTNRWNLQSLLLSAQ
ITGMTVTIKTNACHNGGGFSEVIFR

Clark C., Bast D. J., Sharp A. M., St Hilaire P. M., Agha R., Stein P. E., Toone E. J., Read R. J., Brunton J. L disclose in “Phenylalanine 30 plays an important role in receptor binding of verotoxin-1” Mol. Microbiol. 19:891-899 (1996) mutant F30A (SEQ ID NO: 48).

(F30A):
SEQ ID NO: 48
TPDCVTGKVEYTKYNDDDTFTVKVGDKELATNRWNLQSLLLSAQ
ITGMTVTIKTNACHNGGGFSEVIFR

Perera L. P., Samuel J. E., Holmes R. K., O'Brien A. D. “Identification of three amino acid residues in the B subunit of Shiga toxin and Shiga-like toxin type II that are essential for holotoxin activity.” J. Bacteriol. 173:1151-1160 (1991) and Jemal C., Haddad J. E., Begum D., Jackson M. P. “Analysis of Shiga toxin subunit association by using hybrid A polypeptides and site-specific mutagenesis.” J. Bacteriol. 177:3128-3132 (1995) disclose mutant R33D (SEQ ID NO: 49).

(R33D):
SEQ ID NO: 49
TPDCVTGKVEYTKYNDDDTFTVKVGDKELFTNDWNLQSLLLSAQ
ITGMTVTIKTNACHNGGGFSEVIFR

Jemal et al. above also disclose mutant W34G (SEQ ID NO: 50).

(W34G):
SEQ ID NO: 50
TPDCVTGKVEYTKYNDDDTFTVKVGDKELFTNRGNLQSLLLSAQ
ITGMTVTIKTNACHNGGGFSEVIFR

Bast D. J., Banerjee L., Clark C., Read R. J., Brunton J. L. “The identification of three biologically relevant globotriaosyl ceramide receptor binding sites on the Verotoxin 1 B subunit.” Mol. Microbiol. 32:953-960 (1999) disclose mutant W34A (SEQ ID NO: 51) and G62T (SEQ ID NO: 52).

(W34A):
SEQ ID NO: 51
TPDCVTGKVEYTKYNDDDTFTVKVGDKELFTNRANLQSLLLSAQ
ITGMTVTIKTNACHNGGGFSEVIFR
(G62T):
SEQ ID NO: 52
TPDCVTGKVEYTKYNDDDTFTVKVGDKELFTNRWNLQSLLLSAQ
ITGMTVTIKTNACHNGGTFSEVIFR

Is also known mutant D17E G62T (SEQ ID NO: 53).

(D17E G62T):
SEQ ID NO: 53
TPDCVTGKVEYTKYNDEDTFTVKVGDKELFTNRWNLQSLLLSAQ
ITGMTVTIKTNACHNGGTFSEVIFR

### A. Material and Methods

Recombinant STxB Expression and Purification

The STxB gene was cloned into the pSU108 plasmid, and expression was performed under the transcriptionnal and translational control of the thermoinducible LambdapL/PR promoter. After preparation of periplasmic extracts, these were loaded on a QFF column (Pharmacia) and eluted by a linear NaCl gradient in 20 mMTris/HCl, pH 7.5. Recombinant STxB was eluted between 120 and 400 mM. STxB-containing fractions were dialyzed against 20 mMTris/HCl, pH 7.5, reloaded on a Mono Q column (Pharmacia), and eluted as before. The resulting proteins, estimated to be 95% pure by SDS-polyacrylamide gel electrophoresis, were stored at −80° C. until use.

Generation of a Stable Gb3+CHO Cell Line

The CHO cell line was chosen to generate a cell system for which Gb3-positive and negative cells were available on the same genetic background. CHO cells normally lack expression of lactosylceramide α1,4-galactosyltransferase, the enzyme that catalyzes the conversion of lactosylceramide into Gb3 and its derivatives. To generate a Gb3-positive CHO clone, the Gb3 synthase gene under control of the cytomegalovirus promoter was stably transfected into these cells. The expression of Gb3 and its localization at the plasma membrane was then demonstrated using STxB.

pCDNA3_Gb3_synthase plasmid from J. Wiels lab (Institut Gustave Roussy UMR 8126) was transfected into CHO cells by electroporation. Briefly, 80% confluent cells were trypsinized, centrifuged at 600×g for 5 min and washed once in Phosphate Buffer Saline (PBS). 8×10⁶ cells were resuspended in a 240 μl mix composed of 120 μL electrobuffer mix (Cell projects), 10 μg pCDNA3_Gb3_synthase, 10 μg Salmon Sperm DNA and water. Electroporation was done in a 4 mm gap electroporation cuvette at 0.22 kV with High Cap set at 0.975 μF×1000, and cells were resuspended in 10 mL Dulbecco's modified Eagle's medium: nutrient mixture F-12 (DMEM/F12, Gibco, Life Technologies), supplemented with 10% heat-inactivated fetal bovine serum (Pan Biotech), 0.01% penicillin-streptomycin (Invitrogen), 41 mM L-glutamine and 51 mM sodium pyruvate.

Cells were seeded in a T75 dish, and grown at 37° C. in a 5% CO₂/air atmosphere. After 24 hours, selection medium containing 0.5 mg/mL Geneticin (G418, ThermoFischer) was added and replaced every other day. After 2 weeks of selection, single cell were selected by limited dilution in 96 well plates.

Final selection was performed by FACS sorting, using binding of fluorescently labeled STxB, and intracellular retrograde trafficking of STxB in the selected cell line was controlled by immunofluorescence microscopy. 7×10⁴ Gb3⁺CHO and GB3⁻CHO cells were seeded in P6 plates, and grown overnight at 37° C. under 5% CO₂. After 3 times washes with DMEM/F12 medium at 4° C., cells were incubated for 30 min at 4° C. with 0.1 μM A488-labelled STxB, washed, and either fixed (binding experiments), or incubated for 45 min at 37° C. (retrograde transport experiments). When indicated, the Golgi apparatus was labeled for GM130 (BD transduction laboratories). Images were acquired on an epifluorescence microscope (Leica DM 6000B), and processed with ImageJ software.

GSLs Synthesis Inhibition in HeLa C2TA Cells

DL-threo-1-Phenyl-2-palmitoylamino-3-morpholino-1-Propanol (PPMP) is a glucosylceramide synthetase inhibitor, which was used for the depletion of GSLs.

HeLa cells express the glycosphingolipids Gb3. The HeLa cell clone C2TA homogenously expresses Gb3. HeLa C2TA cells were cultured for 12 days at 37° C. under 5% CO2 in DMEM medium containing 5 μM PPMP (Enzo LifeSciences) with splitting of cells every 3 days. Inhibition of glycosphingolipid synthesis was confirmed by binding of fluorescently labeled STxB on C2TA treated cells analyzed by immunofluorescence and flow cytometry.

Gb3 Synthase Gene Transfection and Selection of a Stable Gb3+CHO Cell Line

Gb3 synthase gene transfection and selection of a stable Gb3+CHO cell line pCDNA3_Gb3_synthase plasmid from J. Wiels lab (Institut Gustave Roussy UMR 8126) was transfected into CHO cells CHO-K1 (ATCC® CCL-61 (trademark)) from Sigma aldrich ref: 85051005-1VL by electroporation. Briefly, 80% confluent cells were trypsinized, centrifuged at 600×g for 5 min and washed once in Phosphate Buffer Saline (PBS). 8×106 cells were resuspended in a 240 μl mix composed of 120 μL electrobuffer mix (cell projects), 10 μg pCDNA3_Gb3_synthase, 10 μg Salmon Sperm DNA and water.

Electroporation was done in a 4 mm gap electroporation cuvette at 0.22 kV with High Cap set at 0.975 μF×1000, and cells were resuspended in 10 mL Dulbecco's modified Eagle's medium: nutrient mixture F-12 (DMEM/F12, Invitrogen), supplemented with 10% heat-inactivated fetal bovine serum (Pan Biotech), 0.01% penicillin-streptomycin (Invitrogen), 41 mM L-glutamine and 51 mM sodium pyruvate. Cells were seeded in a T75 dish, and grown at 37° C. in a 5% CO₂/air atmosphere. After 24 hours, selection medium containing 0.5 mg/mL Geneticin (G418, ThermoFischer) was added and replaced every other day. After 2 weeks of selection, single cell were selected by limited dilution in 96 well plates. Final selection was performed by FACS sorting, using binding of fluorescently labeled STxB, and intracellular retrograde trafficking of STxB in the selected cell line was controlled by immunofluorescence microscopy.

Glycosphingolipids Synthesis Inhibition by PPMP Treatment of C2TA Cells

C2TA cells were cultured for 12 days at 37° C. under 5% CO2 in DMEM medium containing 5 μM PPMP with splitting of cells every 3 days. Inhibition of glycosphingolipid synthesis was confirmed by binding of fluorescently labeled STxB on C2TA treated cells analyzed by immunofluorescence and flow cytometry.

Immunofluorescence Experiment to Confirm Binding and Retrograde Transport of STxB in Stable Gb3⁺CHO Cells

7×10⁴ Gb3⁺CHO and GB3⁻CHO cells were seeded in P6 plates, and grown overnight at 37° C. under 5% CO₂. After 3 times washes with DMEM/F12 medium at 4° C., cells were incubated for 30 min at 4° C. with 0.1 μM A488-labelled STxB, washed, and either fixed (binding experiments), or incubated for 45 min at 37° C. (retrograde transport experiments). When indicated, the Golgi apparatus was labelled for GM130 (BD transduction laboratories). Images were acquired on an epifluorescence microscope (Leica DM 6000B), and processed with ImageJ software.

pHEN2_STxB Phagemid and pHEN2_STxB_Mutant Design & Cloning

The pHEN2_STxB phagemids were designed for the expression of STxB or STxB mutant in fusion with the pIII capside coat protein of bacteriophage M13. These constructs were obtained using the Gibson assembly technique, by recombination between the STxB inserts and the commercially available pHEN2 phagemid. The restriction sites NcoI and Not1 were introduced at the 5′ and 3′ ends of the STxB genes, respectively. The first step consisted in 2 PCRs using appropriate primers to create overhangs of 15 base pairs shared by the plasmid and the insert.

Briefly, these PCR amplifications were done in 50 μL total volume using 10 ng of templates plasmids, 2.5 ng of each primer, 0.5 μl of Phusion (trade mark) High-Fidelity DNA polymerase with appropriate buffer and reagents as described by the manufacturer (New England BioLabs). The PCR program consisted in 5 cycles at 54° C. annealing, followed by 25 cycles at 72° C. annealing temperature. PCR products were purified using a commercial DNA gel extraction kit Cat No 28106 (Qiagen), and were then assembled according to the one-step isothermal DNA assembly method: 0.025 pmol of each DNA fragment were pooled in 5 μl, and 15 μl of home-made assembly master mixture according to Gibson's protocol (500 mM Tris-HCl pH 7.5, 50 mM MgCl₂, 1 mM dGTP, 1 mM dATP, 1 mM dTTP, 1 mM dCTP, 50 mM DTT, 25% PEG-8000 and 5 mM NAD) were added. The mixture was incubated at 50° C. for 1 hour in a thermocycler. 3 μl of Gibson assembly reaction were used for the transformation of DH5alpha thermocompetent E. coli cells, according to the manufacturer's instructions (Invitrogen). Bacteria were cultured on LB plates containing antibiotics. 6 clones were sequenced, and 1 was selected, grown in 2×YT medium with antibiotics, and bacterial plasmid DNA extraction was performed using the QIAprep Spin Miniprep Kit (Qiagen). 50 ng were used for transformation of thermocompetent TG1 E. coli cells (Lucigen) grown in 50 mL 2×YT, 100 ug/mL ampicillin, 1% glucose.

Amber Mutation

The pHEN2_STxB_noAmb phagemid, where the TAG amber stop codon is replaced by a CAG codon was obtained by site-directed mutagenesis using GeneArt Site-directed mutagenesis kit (ThermoFisher Scientific). Appropriate primers were designed and ordered from Eurofins. After transformation of mutagenesis products, 8 clones were sequenced (GATC). One was selected, grown in 2×YT medium containing 100 μg/mL ampicillin. Bacterial plasmid DNA extraction was performed using GIAprep Spin Miniprep Kit (Giagen).

STxB Expression on Phages

50 ng of each phagemids were used for transformation of thermocompetent TG1 E. coli cells (Lucigen) grown in 50 mL 2×YT, 100 μg/mL ampicillin, 1% glucose. Overnight culture of TG1 cells transformed with pHEN2_STxB, pHEN2_STxB_noAmb or pHEN2_STxB_mut were diluted in 10 mL of 2×YT medium, 100 μg/mL ampicillin, 1% glucose, grown from an OD600 of 0.1 to 0.5, infected with 4 μL of 10¹³ Helper phages M13KO7 (NEB) or 40 uL of 10¹² Hyperphage M13 K07ΔpIII (Progen), and incubated for 30 min at 37° C. in a water bath.

Bacteria were then centrifuged for 10 min at room temperature at 3.200×g, and resuspended in 50 mL 2×YT (powder from sigma Aldrich Y2377-250G) without glucose, but containing ampicillin 100 μg/mL and kanamycin at 50 μg/mL. After an overnight growth at 30° C., the cultures were centrifuged 15 min at 3,200×g, and the phage-containing supernatant was collected.

Further isolation of phage particles was obtained by PEG precipitation. 40 mL of supernatant were incubated with 8 mL PEG 8000 30% 2.5M NaCl for 1 hr at 4° C. After 30 min centrifugation at 10,800×g, the pellets were resuspended in 2 mL PBS, and centrifuged once more for 10 min at 13,000×g to remove remaining bacterial residues.

Phage Displaying STxB (Φ_STxB) and STxB_Mut_D18E; G62T (Φ_STxB_Mut) Expression

An overnight culture of TG1 cells transformed with pHEN2_STxB or pHEN2_STxB_mut, was diluted in 10 mL of 2×YT medium, 100 μg/mL ampicillin, 1% glucose, grown from an OD600 of 0.1 to 0.5, infected with 4 μL of 1013 helper phages M13KO7 (NEB), and incubated for 30 min at 37° C. in a water bath. Bacteria were then centrifuged for 10 min at room temperature at 3,200×g, and resuspended in 50 mL 2×YT (powder from sigma Aldrich Y2377-250G) without glucose, but containing ampicillin 100 μg/mL and kanamycin at 50 μg/mL. After overnight growth at 30° C., Φ_STxB or Φ_STxB_mut were harvested by centrifugation for 15 min at 3200×g. Supernatants containing phages were stored for few days at 4° C.

Immunoblotting of Φ_STxB/φ_STxB_noAmb/Φ_STxB_mut

30 μl of TG1 supernatant containing Φ_STxB, Φ_STxB_noAmb or Φ_STxB_mut were heated to 90° C. with 4× denaturing blue loading dye, and loaded on 4-15% gradient polyacrylamide gels (Mini-Protean TGX precast gel, Biorad). After 40 min migration at 150V, and transfer on a nitrocellulose membrane, anti_pIII mouse antibody (1/1,000 dilution, New England Biolabs (NEB)) was used with appropriate anti-mouse HRP secondary antibodies (Beckman Coulter).

Mass spectrometry analysis of STxB_mut_PIII fusion. 30 μl of TG1 supernatant containing Φ_STxB_mut were heated to 90° C. with 5× blue loading dye, and loaded on 4-15% gradient polyacrylamide gels (Mini-Protean TGX precast gel, Biorad). After 40 min migration at 150V, the gel was stained with LabSafe Gel Blue (Biosciences). The corresponding band was cut, and the sample was trypsinized for de novo peptide sequencing.

Immunoblotting of Φ_STxB and Φ_STxB_mut

30 μl of TG1 supernatant containing Φ_STxB or Φ_STxB_mut were heated at 90° C. with 5× blue loading dye, and loaded on 4-15% gradient polyacrylamide gels (Mini-Protean TGX precast gel, Biorad). After 40 min migration at 150V, and transfer on a nitrocellulose membrane anti_pIII mouse antibody (1/1000 dilution, New England Biolabs (NEB)) was used with appropriate anti-mouse HRP secondary antibodies, i.e. from Jackson immunoresearch ref 715-035-151.

Mass Spectrometry Analysis of STxB_Mut_PIII Fusion

30 μl of TG1 supernatant containing Φ_STxB_mut were heated at 90° C. with 5× blue loading dye, and loaded on 4-15% gradient polyacrylamide gels (Mini-Protean TGX precast gel, Biorad). After 40 min migration at 150V, the gel was stained with LabSafe Gel Blue (Biosciences). The corresponding band was cut, and the sample was trypsinized for de novo peptide sequencing.

Binding of Φ_STxB/Φ_STxB_noAmb/STxB_mut to Cells

20 μl of precipitated phages diluted in 200 μl PBS BSA 2% were incubated for 45 min at 4° C. on a wheel for blocking.

For immunofluorescence experiments, 70,000 Gb3⁺CHO, GB3⁻CHO, C2TA, or C2TA_PPMP cells were seeded on coverslips in P24 plates, and grown overnight at 37° C. under 5% CO₂. After 3 washes with DMEM/F12 medium at 4° C., cells were incubated for 45 min at 4° C. in PBS BSA 2% for blocking. After removal of the blocking solution, 200 uL phage solution were added and incubated on cells for 45 min at 4° C. The cells were washed 3 times with PBS BSA 2%, again 3 times with PBS′, and fixed with a solution of 1% PFA for 15 min at room temperature. After neutralization with a solution of 50 mM NH4Cl, cells were washed 3 times with PBS BSA 2% and labeled with appropriate M13 phage coat protein antibody (ThermoFisher), washed again 3 times, labeled with anti-mouse A488-modified antibody, and washed 3 times. Images were acquired on an epifluorescence microscope (Leica DM 6000B), and processed with ImageJ software.
For flow cytometry experiments, 100,000 cells per conditions (Gb3⁺CHO, GB3⁻CHO, C2TA, C2TA_PPMP) were incubated for 45 min at 4° C. in PBS BSA 2%. After this saturation step, cells were centrifuged for 5 min at 600×g, incubated for 45 min at 4° C. with Φ_STxB, Φ_STxB_noAmb or Φ_STxB_mut plus appropriate controls, washed 3 times, and then incubated with mouse anti-M13 antibody (GE) and anti-mouse_488 antibody (Molecular Probes, Invitrogen). STxB was directly labeled with Alexa Fluor 488 NHS ester dyes (ThermoFisher Scientific). Cells were fixed, and flow cytometry was performed. Gating was done on control cells, and readings were recorded in order to get 5,000 events in the gate at fast speed with multiple resuspensions of cells using BD Accuri C6 Cytometer. Data were analyzed using Flowjo software.

Binding of Φ_STxB and Φ_STxB_mut on Cells and Flow Cytometry

20 μl of phages were incubated for 30 min at 4° C. in 100 μl PBS BSA 2%. 100,000 cells per conditions (Gb3⁺CHO, GB3⁻CHO, C2TA, C2TA_PPMP) were incubated for 30 min in PBS-BSA 2%. After this saturation step, cells were centrifuged for 5 min at 600×g, incubated for 45 min at 4° C. with Φ_STxB or Φ_STxB_mut plus appropriate controls, washed 3 times, and then incubated with mouse anti-M13 antibody (GE) and anti-mouse_488 antibody (Molecular Probes, Invitrogen). STxB was directly labeled with Alexa Fluor (registered trade mark) 488 NHS ester dyes (ThermoFisher Scientific). Cells were fixed, and flow cytometry was performed. Gating was done on control cells, and readings were recorded in order to get 5,000 events in the gate at fast speed with multiple resuspensions of cells using BD Accuri (trade mark) C6 Cytometer. Data were analyzed using Flowjo software.

Preparation of Magnetic Liposomes

To generate Gb3-containing liposomes, 150 μL of 5 mg/mL of 1,2-dioleoyl-sn-glycero-3-phosphocholine, 18:1 (8,9-Cis) PC, so-called DOPC (Avanti) were mixed with 100 μL of 1 mg/mL of ceramide trihexosides, or Gb3, (Matreya) in a glass tube. Solvents were removed by evaporation using nitrogen or argon to generate an homogenous lipid film on the wall of the glass tube. Remaining solvents were removed by drying under vacuum for 2 hrs.

The lipid mix was then rehydrated with a solution of 1 mL PBS at 65° C. containing 10 μL iron (II, III) oxide magnetic fluid (7% stock concentration—PlasmaChem). The solution was vortexed for 5 min. 3 cycles of freezing in ethanol/dry ice mix, thawing in water bath at 65° C. and 1 min mixing were performed.

The liposome mixture was then passed 17 times using 1 μm filters through an extruder (Avanti) that was also pre-heated to 65° C. Liposomes were then washed 3 times by recruitment on a magnet, removal of the supernatant and resuspension with a solution of PBS-BSA 2%. Liposomes were directly used or stored at 4° C. for a couple of days maximum. The same procedure was used to generate control liposomes without Gb3.

Characterization of Magnetic Liposomes by Electron Microscopy

Different dilution of magnetic liposomes preparation were made into water and deposed on carbon-coated copper grids that were ionized by glow discharge (at 1-2 mA for 30 s). After drying of the sample, negative staining was performed using uranyl acetate at 2% for 1 min. The grids were washed with water and dried. Images were captures using Tecnai Spirit electron microscope.

STxB Recruitment onto Liposomes

For blocking, a 1 mL solution of PBS-BSA 2%-500 nM STxB was incubated 1 hr at 4° C. on a wheel. This solution was added onto a 200 μL of magnetic liposomes preparations (see below), and incubated on a wheel for 45 min at 4° C. 5 washes were performed in a 15 mL tubes with PBS BSA 2% by collecting the magnetic liposomes on a magnet. 5 additional washes were performed in a new 15 mL tube with PBS. STxB recruitment was analyzed by immunobloting and FACS.

In the first case, liposomes were resuspended in 150 uL PBS to which 50 μL denaturing blue loading dye was added. The solution was boiled for 10 min at 90° C., and 50 μL were loaded on a 4-15% gradient polyacrylamide gel (Mini-Protean TGX precast gel, Biorad). After 40 min migration at 150V, and transfer on a nitrocellulose membrane, anti-STxB (13C4) antibody was used with appropriate anti-mouse HRP secondary antibodies.

For the FACS analysis, Alexa_488 labeled STxB was used. The liposomes were resuspended after washed in 300 μL and passed through a flow cytometer (BD Accuri C6, BD Biosciences).

Phage Recruitment onto Magnetic Liposomes

For blocking, 100 μL of freshly produced and precipitated phages (around 10¹² phages) were diluted into 1 mL final volume of PBS-BSA 2% and incubated 1 hr at 4° C. on a wheel. This solution was added onto 200 uL of magnetic liposomes preparations (see below), and incubated on the wheel for 45 min at 4° C. 5 washes were performed in a 15 mL tubes with PBS BSA 2% by collecting the magnetic liposomes on a magnet. 5 additional washes were performed in a new 15 mL tube with PBS. The phage recruitment was analyzed by immunobloting and FACS.

In the first case, the liposomes were resuspended in 150 μL PBS to which 50 μL denaturing blue loading dye was added. The solution was boiled for 10 min at 90° C., and 50 uL were loaded on a 4-15% gradient polyacrylamide gel (Mini-Protean TGX precast gel, Biorad). After 40 min migration at 150V, and transfer on a nitrocellulose membrane, anti_pIII mouse antibody (1/1000 dilution, New England Biolabs (NEB)) was used with appropriate anti-mouse HRP secondary antibodies.

For the FACS analysis, the liposomes were resuspended 1 mL solution of PBS-BSA 2% with anti-M13 antibody and incubated on a wheel for 45 min at 4° C. 3 washes were performed before incubation with Alexa488 labeled anti-mouse antibody. After 3 more washes, the liposome solution was passed through a flow cytometer (BD Accuri C6, BD Biosciences).

Simulation of Phage Display Selection on Magnetic Liposomes

10¹² Φ_STxB_mut were mixed with 10⁸ Φ_STxB (ratio of 1/10 000) in 1 mL PBS-2% BSA, incubated for blocking 1 hr at 4° C. on a wheel. The solution was added to 200 uL of magnetic liposomes preparations (see below) and incubated on a wheel for 45 min at 4° C. 10 washes were performed in a 15 mL tubes with PBS BSA 2% by collecting the magnetic liposomes on a magnet. 10 additional washes were performed in a new 15 mL tube with PBS.

Phages were eluted using 1 mL of a solution of 50% Trypsin in PBS at 37° C. for 10 min. After addition of 500 uL SVF, 750 uL of the solution was used to infect 10 mL TG1 bacteria (DO=0.5) for 30 min at 37° C. without agitation. 100 uL was used to prepare several dilutions of the bacterial solution (10-1, 10-2 and 10-3), which were then seeded on 2×TY agar ampicillin 1% glucose plates which were incubated overnight at 37° C.

The next day, 24 clones were collected, and grown overnight at 37° C. in 5 mL 2×TY ampicillin 1% glucose solution. Bacterial plasmid DNA was extracted using the QIAprep Spin Miniprep Kit (Qiagen) and sequenced (GATC).

Design of the STxB Variant Library

The twenty positions—Thr1, Asp16, Asp17, Asp18, Thr21, Glu28, Leu29, Phe30, Thr31, Asn32, Arg33, Trp34, Asn35, Thr54, Asn55, Ala56, Gly60, Gly62, Phe63, Ser64—involved in the binding of STxB to Gb3 (Ling et al., 1998) were chosen for the creation of the combinatorial library.

To reach a total population of 1.5×10¹⁰ variants, three to four alternative amino acids are possible at each of the twenty positions, as described herein. The alternative amino acids were selected with the help of the pfam platform website for sequence alignment. For this, 286 STxB homologues from Uniprot database, and 211 homologues from NCBI database were aligned, and results compiled with the Hidden Markov Model (HMM) logo generation software. The most represented amino acids were chosen in order to maximize the chance of getting properly folded pentameric STxB variants.

The library was then synthetized by the timer oligonucleotide synthesis (TRIM technology, by GeneArt), amplified, and sub-cloned into the proper phen2 expression system (GeneArt), to reach a final diversity of 10⁹ clones.

Characterization of the Library

The content of the library was characterized by sequencing by both, by the GeneArt company and in the laboratory (not shown). Different dilutions of the clones obtained from GeneArt were plated on 2×YT 100 μg/mL ampicillin, glucose 1%, agar plate. 96 clones were picked and sequenced using appropriate primers (GATC). Sequences were processed and aligned using CLC Workbench software.

Phage Library Amplification

100 μL of TG1 bacteria from GeneArt (1.68×10¹¹ clones/mL) were grown in 250 mL 2×YT, 100 μg/mL Ampicillin, 1% glucose at 37° C. to reach DO=0.5. 75 mL of culture were infected with 6×10¹¹ M13 helper phage and incubated 30 min at 37° C. without agitation. The solution was then centrifuged for 20 min at 3,200×g at room temperature. The pellet was resuspended in 1.5 L of 2×YT, 100 μg/mL ampicillin, 100 μg/mL kanamycin and grown overnight at 30° C.

500 mL were centrifuged at 10,800×g at 4° C. ⅕^{th} volume of 30% PEG, 2.5MNaCl solution were added to the supernatant and incubated 1 hr at 4° c. without agitation. The solution was then centrifuged for 30 min at 10,800×g at 4° C., and the pellet was resuspended in 40 mL sterilized deionized water. 8 mL of 30% PEG, 2.5M NaCl were added, and the solution was incubated again for 30 min at 4° C. The solution was finally centrifuged for 30 min at 10,800×g at 4° C., and the pellet was resuspended in 16.5 mL PBS 15% glycerol.

After a last centrifugation step at 13,000×g at 4° C., the solution was aliquoted and stored at −80° C. 5 μL were used to titer the phage concentration by infection TG1 bacteria with different dilutions of the phage stock.

Phage Display Selection of Gb3 Binders

Day 1: Magnetic Liposomes Preparation

1 mL of 1 μm Gb3⁺ and Gb3⁻ magnetic liposome solution was produced as described previously. Liposomes were then washed 3 times by recruitment on a magnet, removal of the supernatant and resuspension with a solution of PBS-BSA 2%. The liposomes solution was divided in two, resuspended in 1.5 mL PBS-2% BSA, and incubated overnight at 4° C. on the wheel.

TG1 bacteria were grown in 50 mL M9 minimal medium complemented with 2 μM MgSO₄, 1% glucose, 0.1% thiamine, overnight at 37° C. This culture was kept at 4° C., and used for a maximum of 3 weeks.

Day 2: Phage Display Selection on Liposomes

One aliquot of the STxB library stock was thawed. 100 μL was diluted in 900 μL PBS-2% BSA for 1 hr at 4° C. on a wheel. Two 15 mL tubes were coated with PBS-2% BSA on ice. Gb3-liposomes were recruited on a magnet for 10-15 min at 4° C., and the 1 mL phage solution was added and incubated on a wheel at 4° C. for 1 hr.
In parallel, 3 solutions of 15 mL 2×YT 1% glucose inoculated with 1/50, 1/100 and 1/200 TG1 stock solution were incubated at 37° C. with agitation.

The liposomes were then collected, and the supernatant was added to the second solution of Gb3⁻ liposomes, incubated 1 hr at 4° C. on a wheel. Liposomes were collected, and the supernatant was removed. After these two depletion steps, the phage supernatant was added to Gb3⁺ magnetic liposomes, and incubated 1 hr at 4° C. on a wheel. The liposomes were then collected on a magnet, and resuspended in 10 mL PBS-2% BSA in the first pre-coated 15 mL tube. 10× washes were performed alternating 5 min recruitment on a magnet at 4° C. and resuspension in 10 mL cold PBS-2% BSA solution. The liposomes were transferred to the second pre-coated 15 mL tube and 5× washes in cold PBS-2% BSA and 5× washes in cold PBS were performed.

Finally, the liposomes were collected on a magnet, and phages were eluted using 1 mL of a solution of 50% Trypsin in PBS at 37° C. for 10 min. After addition of 500 uL SVF, 750 uL of the solution was used to infect 10 mL TG1 bacteria (D0=0.5) for 30 min at 37° C. without agitation. 100 μL was used to make several dilutions of the bacterial solution (10⁻¹, 10⁻² and 101. Bacteria were plated on 2×TY agar plates with 100 μg/mL ampicillin 1% glucose, which were incubated overnight at 37° C. to calculate the output concentration of phages.

The remaining 10 mL TG1 solutions was centrifuged and resuspended in 1.8 mL 2×YT. 600 μL were plated on 3 large 2×YT agar plates containing 100 μg/mL ampicillin 1% glucose, and grown overnight at 37° C.

Day 3: Amplification of Selected Phages

The output and the input concentration of phages were calculated and the clones on the three large agar plates were collected in 10 mL 2×YT, 30% glycerol, the bacterial concentration was measured and the solution was stored at −20° C. consisting in the Bacterial stock R1.

To amplify the phages, an aliquot of bacterial stock R1 was diluted in 100 mL 2×YT 100 μg/mL ampicillin 1% glucose to reach DO=0.05, and grown to reach DO=0.5. 10 mL were infected with 8×10¹⁰ helper phages, and incubated for 30 min at 37° C. without agitation. The 10 mL solution was then centrifuged for 20 min at 3,200×g at room temperature. The pellet was resuspended in 50 mL of 2×YT, 100 μg/mL ampicillin, 100 μg/mL kanamycin, and grown overnight at 30° C.

40 mL were centrifuged at 10,800×g at 4° C. ⅕^{th} volume of 30% PEG, 2.5 MNaCl solution were added to the supernatant, and incubated 1 hr at 4° C. without agitation. The solution was then centrifuged for 30 min at 10,800×g at 4° C., and the pellet was resuspended in 2 mL cold PBS. After a last centrifugation step at 13,000×g at 4° C., 100 μL were used for the second round of selection, and 5 uL for the calculation of the input concentration.

Following the same procedure, 3 rounds of selection on liposomes were performed. A final round of selection was performed on CHO cells.

R4 Selection on CHO Cells:

20×10⁶ Gb3⁻CHO and 10×10⁶ Gb3+CHO cells were trypsinized, and incubated in 10 mL PBS 2% BSA for 1 hr at 4° C. on a wheel. 100 uL of amplified phages from R3 were diluted in 1 mL PBS 2% BSA, and incubated for 1 hr at 4° C. on a wheel. 10×10⁶ Gb3⁻ cells were centrifuged for 5 min at 600×g at 4° C., resuspended in 1 mL phage solution, and incubated for 1 hr at 4° C. on a wheel. Cells were centrifuged for 5 min at 600×g at 4° C. The supernatant was used to resuspended the second half of the Gb3-CHO for a second step of depletion of 1 hr at 4° C. on a wheel.

Cells were centrifuged at 600×g at 4° C., and the supernatant was collected. The 10 mL solution of Gb3+CHO cells were centrifuged at 600×g at 4° C., resuspended with the 1 mL solution of depleted phages, and incubated 1 hr at 4° C. on a wheel. 10× washes were performed consisting of cycles of centrifugation at 600×g at 4° C. and resuspension in 10 mL cold PBS 2% BSA.

A final wash in PBS was performed, and phages were eluted and used to infect TG1 bacteria as described previously. One day later, the bacteria were collected from the agar plates and stored in 2×YT 30% glycerol at −20° C. (Bacterial stock R4 CHO).

Characterization of Gb3 Binders

50 μL of Bacterial stock R4 CHO were centrifuged, and phamegid DNA was extracted using the QIAprep Spin Miniprep Kit (Qiagen). 5 ng of DNA preparation were used to transform competent TG1 cells, which were seeded on 2×YT agar plates containing 100 μg/mL ampicillin, 1% glucose, and grown overnight at 37° C. 96 clones were picked and inoculated in 400 μl 2×YT 100 μg/mL ampicillin, 1% glucose, grown overnight at 37° C. The 96 clones were stored in 400 uL 2×YT 30% glycerol at −20 C.

5 μL were used for sequencing (GATC). The sequences were analyzed and aligned using CLC workbench software.

a) Phage Candidate Screening by Flow Cytometry on HeLA C2TA Cells

### Expression and Production:

In 96 deep well plates, 2 μL of each of the 96 clones were used to inoculate 200 μL of 2×YT solution containing 100 μg/mL ampicillin 1% glucose, and grown to reach DO=0.5. Two wells were used to grow appropriate controls (Φ_STxB and Φ_STxB_mut). 1.5×10⁹ helper phages were used to infect each well, and the plates were incubated at 37° C. for 30 min without agitation. Plates was then centrifuged, and the bacteria were resuspended with 600 uL 2×YT 100 μg/mL ampicillin, 50 μg/mL Kanamycin, and grown overnight at 30° C. with agitation. Plates were centrifuged at 3,200×g for 30 min at 4° C.

### Flow Cytometry Experiment:

200 μL of supernatant was used for the binding experiment. 100,000 C2TA, C2TA_PPMP cells per conditions were incubated at 4° C. for 45 min in PBS BSA 2%. After this saturation step, cells were centrifuged for 5 min at 600×g, incubated for 45 min at 4° C. with 200 μL of phage supernatant, washed 3 times, and then incubated with mouse anti-M13 antibody (GE) and anti-mouse_488 antibody (Molecular Probes, Invitrogen). Cells were fixed, and flow cytometry was performed. Gating was done on control cells, and readings were recorded in order to get >5,000 events in the gate at fast speed using BD Accuri C6 Cytometer. Data were analyzed using Flowjo software.

b) Binding of Phage Candidates and Characterization by Immunofluorescence on CHO Cells

### Expression and Production:

In 24 deep well plates, 2 μL of each of the 14 selected clones were used to inoculate 200 μL of 2×YT solution containing 100 μg/mL ampicillin 1% glucose, and grown to reach DO=0.5. Two wells were used to grow appropriate controls (Φ_STxB and Φ_STxB_mut). 1.5×10⁹ helper phages were used to infect each well, and plates were incubated at 37° C. for 30 min without agitation. Plates were then centrifuge, and the bacteria were resuspended with 600 μL 2×YT 100 μg/mL ampicillin, 50 μg/mL Kanamycin, and grown overnight at 30° C. with agitation. Plates were centrifuged at 3,200×g for 30 min at 4° C.

### Immunoblotting:

25λL of 4× denaturing blue loading dye was added to 75 μL of supernatant of each phage candidate, and the solution was boiled for 10 min at 90° C. 50 μL were loaded on a 4-15% gradient polyacrylamide gel (Mini-Protean TGX precast gel, Biorad). After 40 min migration at 150V, and transfer on a nitrocellulose membrane, anti_pIII mouse antibody (1/1000 dilution, New England Biolabs (NEB)) was used with appropriate anti-mouse HRP secondary antibodies.

### Immunofluorescence:

200 μL of supernatant were used for the binding experiment. 70,000 Gb3⁺CHO and GB3⁻CHO cells were seeded on coverslips in P24 plates, and grown overnight at 37° C. under 5% CO₂. After 3 times washes with cold PBS 2% BSA, cells were incubated for 45 min at 4° C. in PBS BSA 2% for blocking. After removal of the blocking solution, the 200 μL phage supernatant solution was added and incubated on cells for 45 min at 4° C. The cells were washed 3 times with PBS BSA 2% and again 3 times with PBS′, and fixed with a solution of 1% PFA for 15 min at room temperature. After neutralization with a solution of 50 mM NH4Cl, cells were washed 3 times with PBS BSA 2%, labeled with appropriate M13 Phage coat protein antibody (ThermoFisher), washed again 3 times, labeled with anti-mouse A488-labeled antibody, and washed 3 more times. Images were acquired on an epifluorescence microscope (Leica DM 6000B), and processed with ImageJ software.

### B. Results

### M13 Bacteriophages Displaying STxB (Φ_STxB) are Able to Specifically Bind Gb3 Positive Cells

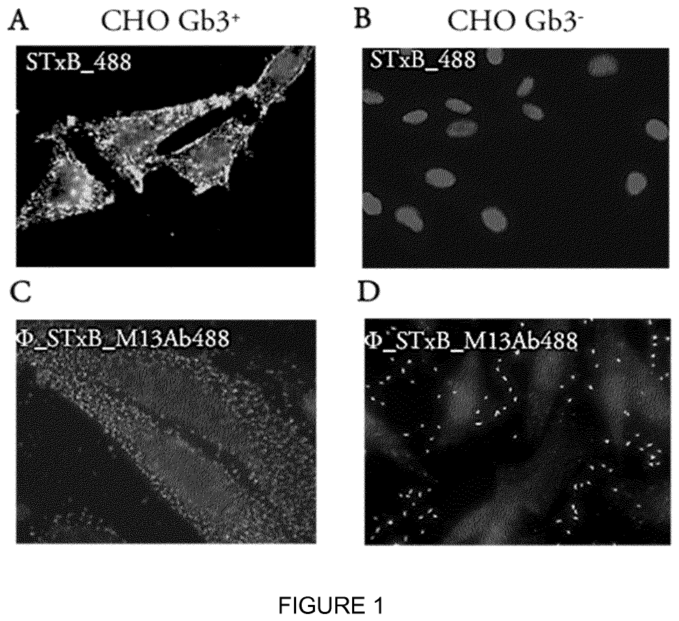

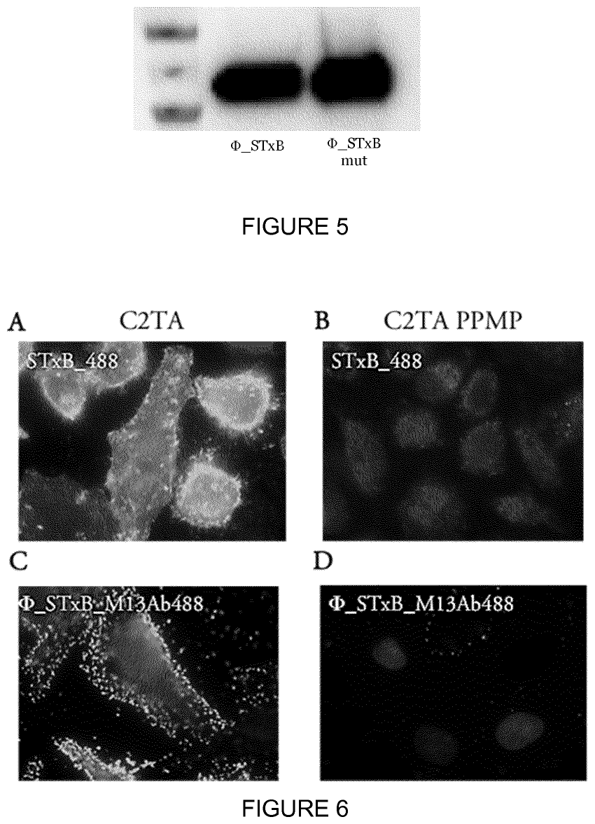

Displaying STxB and STxB mutant (STxB_mut_D18E; G62T) on M13 bacteriophages The STxB gene was fused to the one coding for the coat protein pIII of M13 bacteriophage to drive the expression of a corresponding fusion protein (FIG. 1) in TG1 E. coli bacteria. The proper expression into the supernatant of TG1 E. coli bacteria was tested by immunoblotting. A positive band for antibody against pIII could be detected at the size corresponding to the STxB-pIII fusion protein, demonstrating that this protein was indeed expressed (FIG. 5).

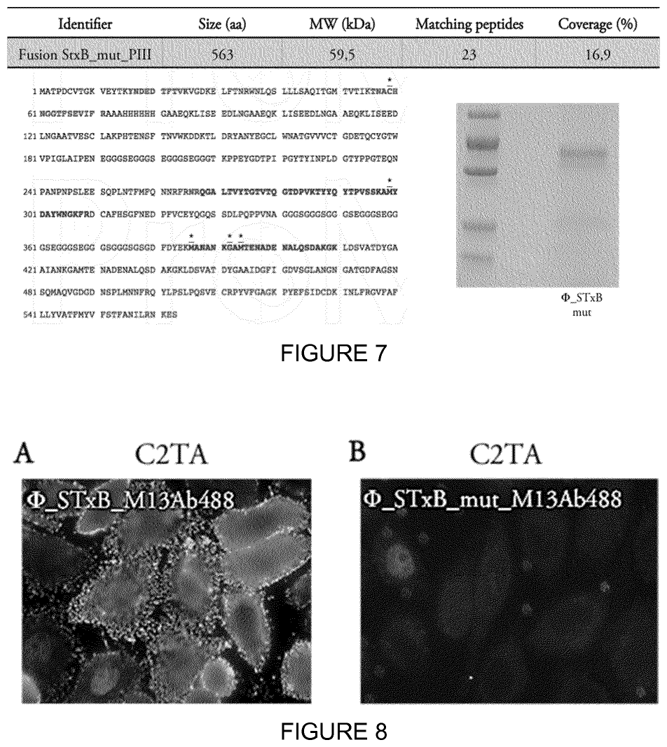

A mutant of STxB (STxBmut-D18E; G62T)-SEQ ID NO: 32, which is not able to bind Gb3 anymore, was also presented on M13 bacteriophage. Correct expression into TG1 supernatant was also confirmed by immunoblotting and mass spectrometry analysis which revealed 13 matching peptides confirming the presence of the mutations and the fusion to the PIII protein (FIG. 7).

The concentration and the infection properties of those phages were tested by a titration assay, where different dilutions of phage preparation were used to infect TG1 bacteria.

Stable and functional expression of globotriaosylceramide (Gb3) at the plasma membrane of Chinese Hamster Ovarian (CHO) cells

The CHO cell line was chosen to generate a cell system for which Gb3-positive and negative cells were available on the same genetic background. CHO cells normally lack expression of lactosylceramide α1,4-galactosyltransferase, the enzyme that catalyzes the conversion of lactosylceramide into Gb3 and its derivatives. To generate a Gb3-positive CHO clone, the Gb3 synthase gene under control of the cytomegalovirus promoter was stably transfected into these cells. The expression of Gb3 and its localization at the plasma membrane was then demonstrated using a natural Gb3 ligand, the B-subunit of Shiga Toxin (STxB).

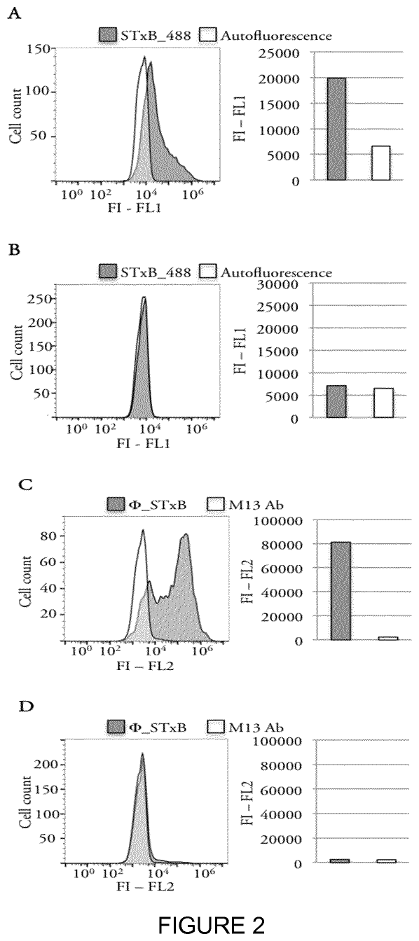

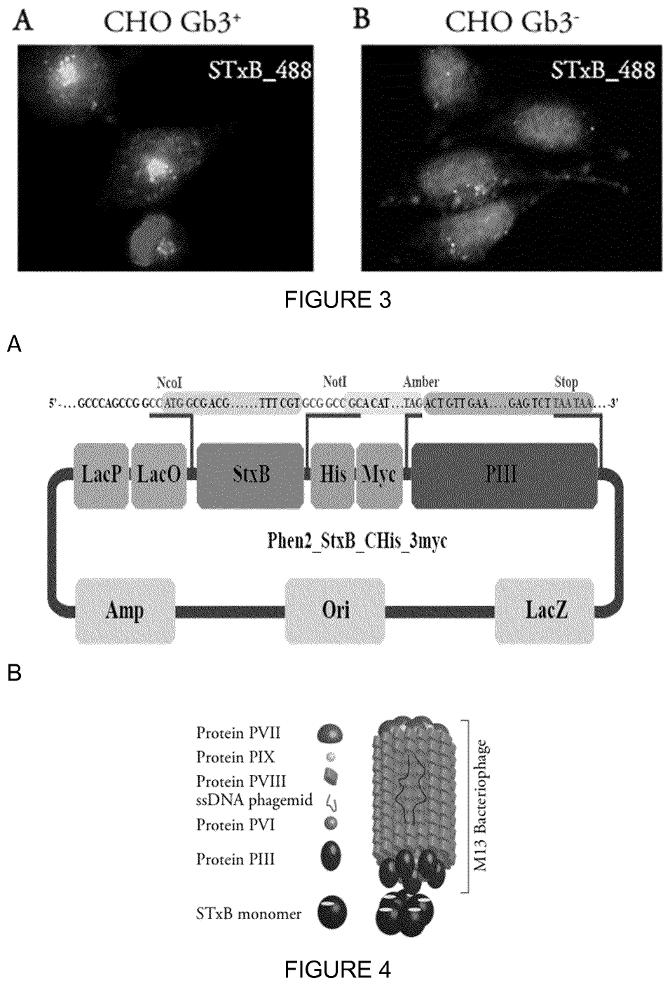

Clear binding of STxB was observed by immunofluorescence when the protein was incubated with CHO cells that had been transfected with the Gb3 synthase gene (Gb3⁺CHO), when compared to non-Gb3 synthase-transfected control cells (Gb3⁻CHO) (FIG. 1). Flow cytometry experiments confirmed this result by showing that the mean fluorescence intensity was shifted to higher values only on Gb3⁺CHO cells (FIG. 2). Retrograde trafficking of STxB to the Golgi apparatus has also been observed (FIG. 3), demonstrating that Gb3 was functional in these Gb3⁺CHO cells.

### Specific Binding of Phage Displaying STxB on Gb3 Positive Cells

Gb3⁺CHO and Gb3⁻CHO cells were incubated with the phage-STxB conjugate (Φ_STxB) for 45 min on ice (no endocytosis). A clear binding was observed to Gb3⁺CHO cells, but not to Gb3⁻CHO cells, when analyzed by immunofluorescence microscopy (FIG. 1).

The binding Φ_STxB to cells was further analyzed by flow cytometry. After incubation with Φ_STxB, a shift in the mean fluorescence intensity was observed between Gb3+CHO and Gb3⁻CHO cells, demonstrating that STxB was functionally expressed at the surface of the phages (FIG. 2).

In order to confirm the specific binding of this Φ_STxB on Gb3, the same binding experiments were performed on C2TA cells, which naturally expressed Gb3. The loss of binding by treatment with PPMP, a specific inhibitor of glycosphyngolipids synthesis, strongly suggests the specific recognition of the Gb3 targets (FIG. 6).

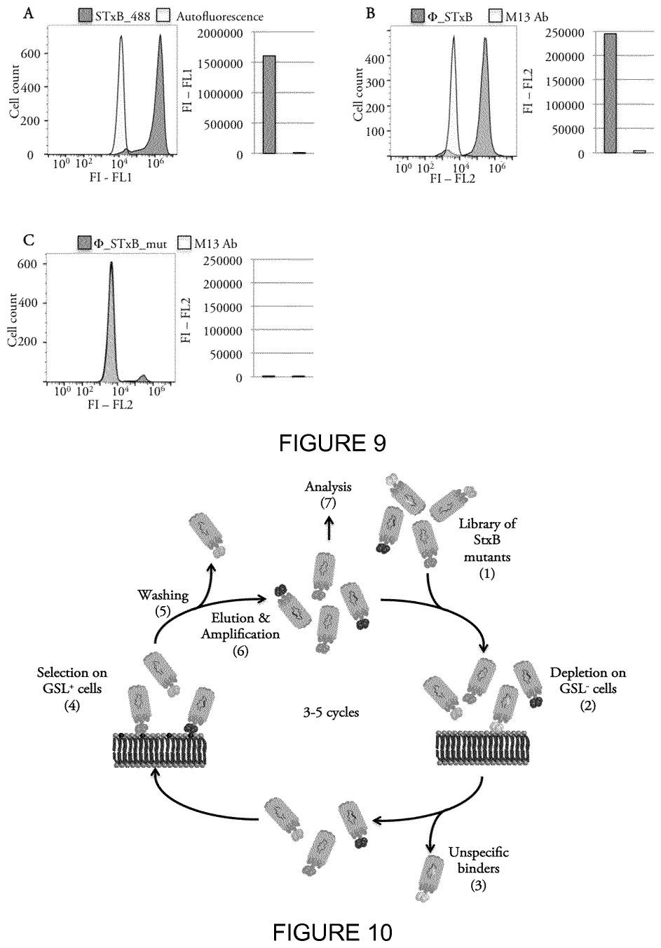

Finally, the binding of of STxB mutant (STxB_mut-D18E; G62T) presenting phages was analyzed by immunofluorescence microscopy and flow cytometry (FIGS. 8-9) and did not show any significant binding. It thus confirmed that displaying of STxB on the M13 bacteriophage specifically drives its binding on Gb3⁺ cells.

These data demonstrate that STxB is functional at the phage surface, and its binding activity is unperturbed. Inventors proposed to exploit this configuration to produce screening libraries in which the STxB gene is systematically mutated and the phages express peptides of the invention that gain binding activity against glycosphingolipids to which commonly known STxB moieties do not bind naturally (FIG. 10).

### Conformational Study of STxB Presented on the M13 Bacteriophage

Part of the conception of the present invention, the actual required conformation of STxB on a phage was investigated. Indeed, STxB molecules are only found in solution as a pentamer. Each phage particles are composed of five pIII proteins that are used for the display of the protein.

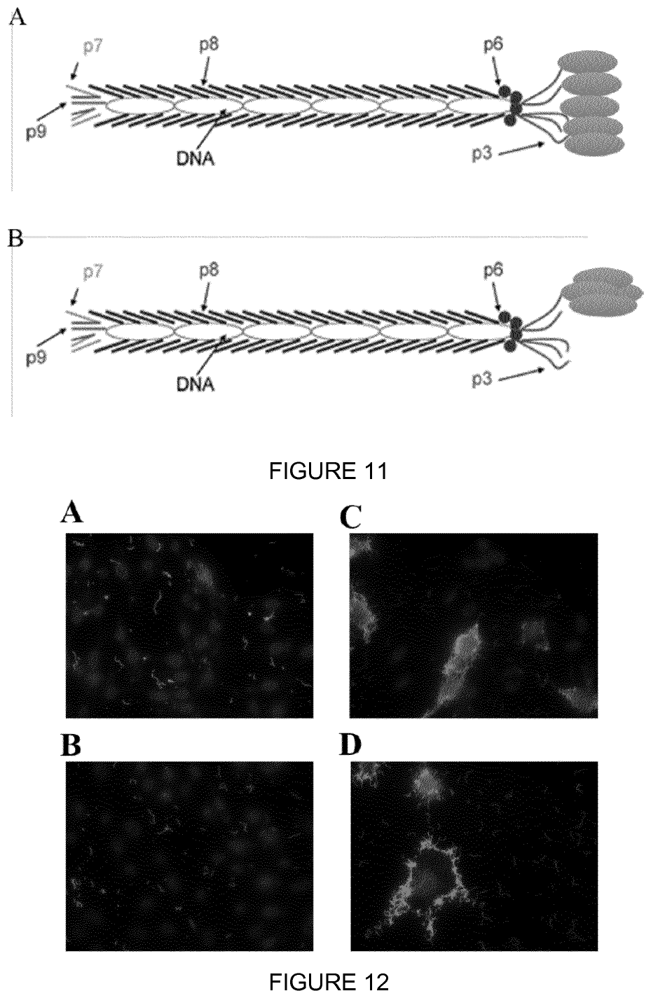

The inventors considered how monomers could be presented on phages. Two hypothesis were envisioned (FIG. 11):
Five STxB monomers, each of them fused with one pIII protein of the phage, were able to pentamerize. Only one pentamer of STxB could be then displayed on a phage particle (FIG. 11.A)
One STxB monomer in fusion with the pIII protein was able to pentamerize with 4 others free STxB monomer in the periplasm of the bacteria during the assembly of the phage particles. One to five STxB pentamer could then be displayed on a phage particle (FIG. 11.B).

Indeed, the presence of an amber stop codon between the STxB gene and the one of the pIII has been designed to allow for the expression of either free STxB protein or STxB_pIII fusion protein with a ratio of approximately 50% each (see Experimental Section herein). Furthermore, to determine the physical rationale underlying the possibility to preform the present invention, two types of helper phage have been used. Standard helper phage possesses in their genome the gene encoding for pIII. The production of pIII protein in the bacteria results from both the expression of the viral pIII gene and the bacterial gene. A phage variant called hyperphage doesn't have this viral pIII gene (Rondot, Koch, Breitling, & Dübel, 2001). The expression of the pIII protein in this case results only from the expression of the bacterial pIII gene. Where the use of the amber stop codon could results in the expression of non-fused STxB proteins, the use of standard helper phage could results in the presentation of non-fused pIII protein.

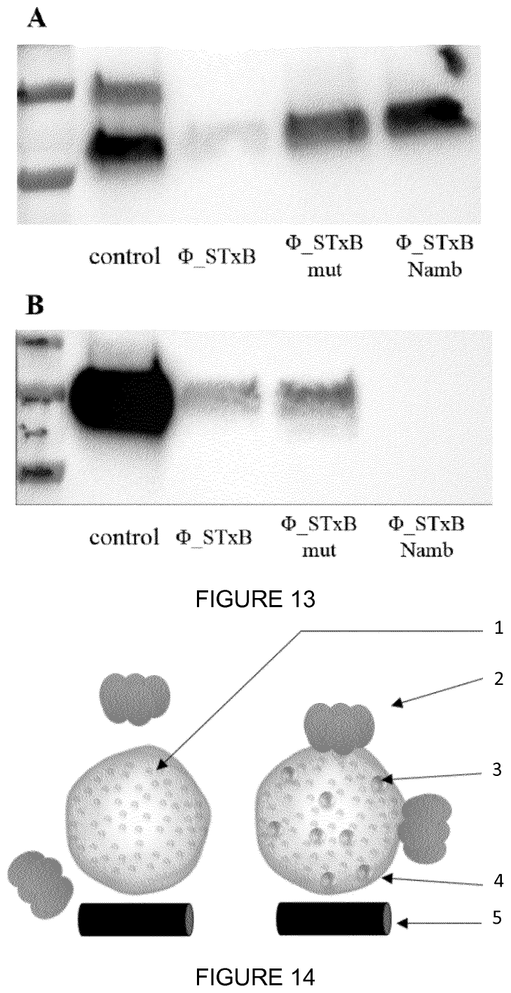

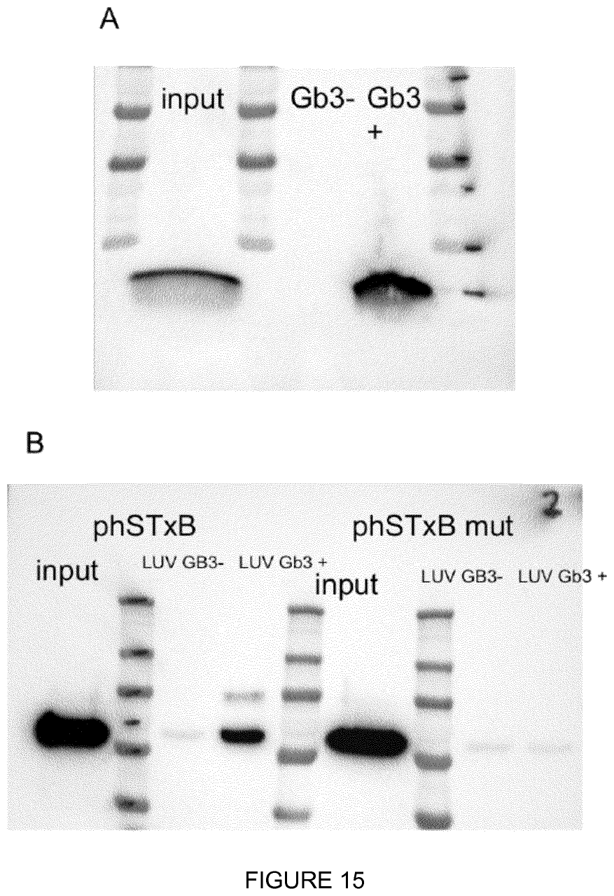

By using a combination of expression systems were STxB monomers could be expressed either at all time (without the amber stop codon), either only from time to time (presence of both fused and non-fused forms) (with the amber stop codon) in fusion with the pIII protein and a combination of helper phage particles that could or could not present non-fused pIII protein on the phage capsid, the inventors have been able to show that the second hypothesis was correct. Indeed, the production of fully fused STxB_pIII resulted in a phage preparation, both with the use of hyper and helper phages, which was not able to bind Gb3 positive cells anymore (The correct expression of phage particles was confirmed by immunoblotting and the binding by immunofluorescence FIG. 12-13). Interestingly, in the case where no amber codon was present in the expression system, combined with the use of hyperphage, no pIII proteins could be detected by immunoblotting. The inventors therefore achieved the definition of an optimal expression system, enabling provision of an optimal presentation of STxB on M13 bacteriophages.

These data demonstrate the an ingenious design from the inventors to enable production of STxB properly folded and functional when displayed on the M13 bacteriophage, potentially driving the binding of the phage particle to Gb3 positive cells.

### Magnetic Liposome-Based Phage Display

As a proof of concept, a strategy increasing the chances that GSLs are presented in their “physiological environment of the lipid bilayer has been devised, using magnetic liposomes.

### Generation of GSL-Containing Magnetic Liposomes

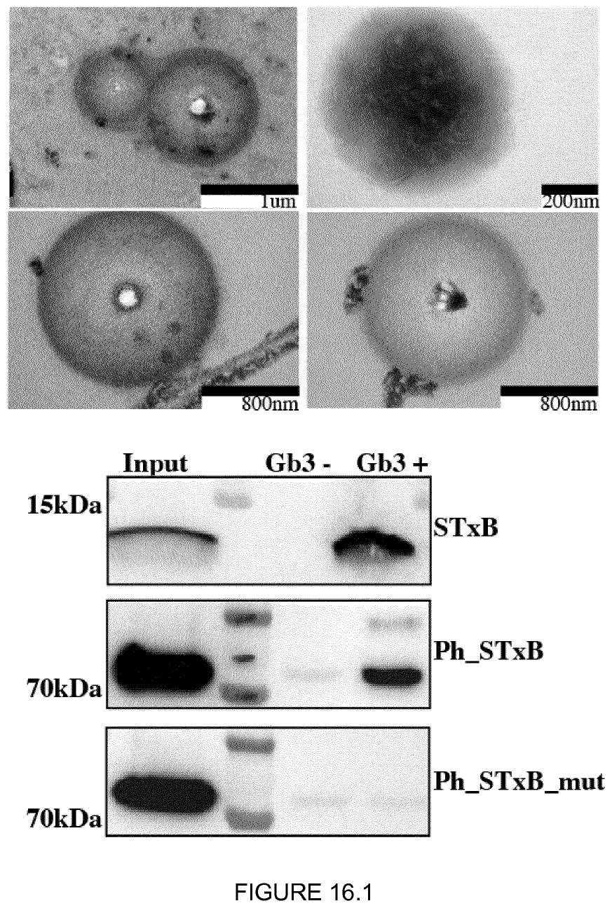

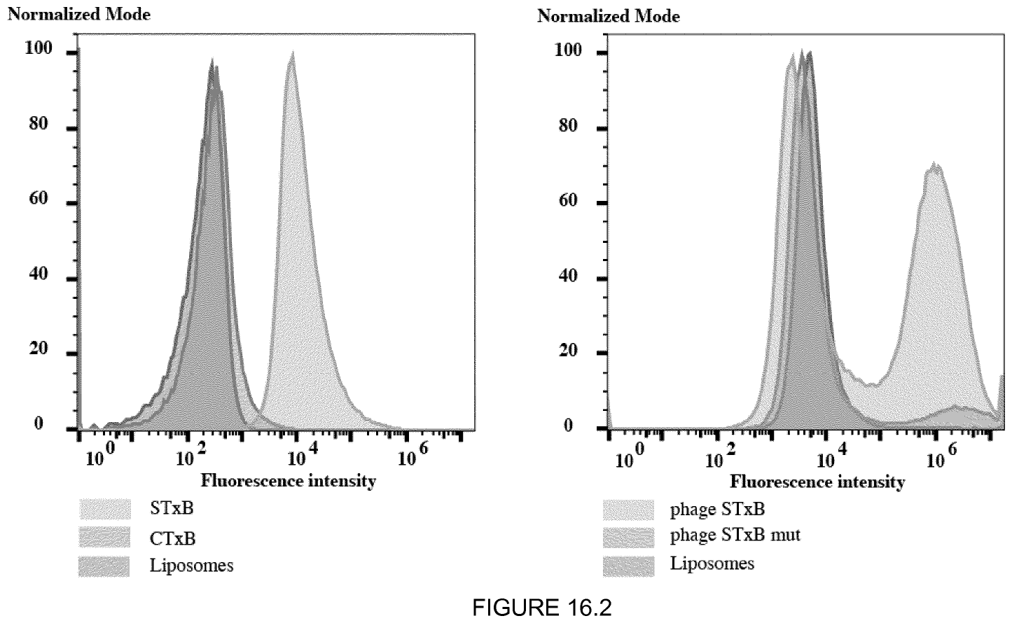

Magnetic DOPC-based liposomes of 1 μm diameter containing Gb3 (or not) were generated. By electron microscopy, rounded and electron dense structures could be observed (FIG. 16) confirming the proper formation of liposomes and incorporation of magnetic nanoparticles. The main advantage of these magnetic liposomes is that they can be recruited using strong magnets, avoiding long and fastidious centrifugations steps.

Specific Recruitment of STxB and Φ_STxB onto Liposomes

In order to confirm the potential of Gb3-containing magnetic liposomes for phage display selection, STxB or Φ_STxB recruitment was analyzed by immunobloting or flow cytometry. STxB and Φ_STxB were only recruited onto Gb3-containing liposomes, which was demonstrated by the presence of a pIII_STxB band on gels (FIG. 16), and the shift in fluorescence by flow cytometry (FIG. 16). No significant recruitment could be observed on Gb3-negative liposomes (FIG. 16). Furthermore, no significant recruitment of Φ_STxB_mut could be observed, neither on Gb3-negative nor on Gb3-positive liposomes (FIG. 16). Likewise, the B-subunit of cholera toxin (CtxB) was not recruited on liposomes either (FIG. 16), demonstrating that recruitment of STxB and Φ_STxB occurred through their binding to Gb3.

### Simulation of Phage Display Selection on Magnetic Liposomes

To finally assess the power of magnetic liposomes for phage display selection, a single round of selection was performed with a mixture of Φ_STxB and Φ_STxB_mut at a ratio of 1 to 10,000. After 2 depletion steps on Gb3-negative magnetic liposomes, the remaining phages were applied to the Gb3-positive magnetic liposomes. After extensive washes, phages were collected and used to infect TG1 bacteria. Sequencing was performed on the clones obtained after selection and a ratio of 1 to 24 between Φ_STxB and Φ_STxB_mut has been assessed. This demonstrated the potential of magnetic liposomes for phage display selection of GSL binders from a protein library.

### Selection of Gb3-Specific STxB Mutants by Phage Display

As a further step in the proof of concept exploration of our phage display selection strategy of STxB variants, a complete screening was performed on Gb3-containing magnetic liposomes, using a library of 1.46×10¹⁰ variants of STxB.

### Design of a STxB Variant Library

STxB contains 3 Gb3 binding sites per B-fragment monomer. Twenty positions out of the sixty-nine (28.9% of the sequence) of the STxB monomer were previously shown to be involved in the binding of the Gb3 (Ling et al., 1998). These 20 positions were chosen for the creation of a combinatorial library. Three to four amino acids can be chosen at each of the twenty positions for a total number of 1.46×10¹⁰ variants, as described herein. The alternative amino acids were selected with the help of the pfam platform website (http://pfam.xfam.org/) for sequence alignment. For this, 286 STxB homologues from Uniprot database, and 211 homologues from NCBI database were aligned, and results compiled with the Hidden Markov Model (HMM) logo generation tools from the platform. The most represented amino acids were chosen in order to maximize the chance of obtaining properly folded pentameric STxB variants. The library was then synthetized by the trimer oligonucleotide synthesis (TRIM technology, by GeneArt), amplified, and sub-cloned into the proper phen2 expression system to obtain a library of fusion proteins between STxB variant and the pIII coat protein of the M13 phage. The total number of transformants was 1.03×10⁹ cfu. The content of the library was characterized by sequencing (not shown). The library of phages was produced and stored in 30% glycerol at −80° C. The phages were produced at a final concentration of around 10¹² phages/m L.

### Library Characterization

The quality and the diversity of the library were validated by Sanger sequencing. 96 colonies from transformation plates were picked and sequenced (GeneArt). 71 of the 96 clones (73%) contained correct sequences. In the 71 sequences, all the desired mutations were found with a minimum of 8% occurrence for the amino acid F30. The remaining clones were either not showing clean sequencing data, or incorrect sequences (insertions, deletions and substitutions). To confirm these data, we also sequenced 96 clones ourselves. 76 out of the 96 clones (79%) contained correct sequences. All the desired mutations were also found with a minimum of 3% occurrence for the amino acid G62, all the other mutations showing an occurrence over 10%. The remaining clones were either not showing clean sequencing data, or incorrect sequences (insertions, deletions or substitutions).

### Phage Display Selection of Gb3 Binders

The first selection was performed against Gb3 in order to assess the potential for selecting non natural sequences of STxB that keep their ability to bind Gb3. Three rounds of selection were performed on magnetic liposomes, where each round consisted in two steps of depletion on Gb3-negative liposomes, to remove unspecific binders, followed by 1 step of selection on Gb3-positive liposomes (FIG. 10). 10¹² phages were used at each round, leading to an output after selection of approximately 107 phages. A final round of selection was performed on Gb3+CHO cells with two steps of depletion on Gb3-CHO, which led to a final output of 105 phages.

96 clones were picked, sequenced and analyzed for their specific binding to GSLs by flow cytometry on HeLa C2TA cells and HeLa C2TA cells treated with PPMP (inhibition of GSL synthesis).

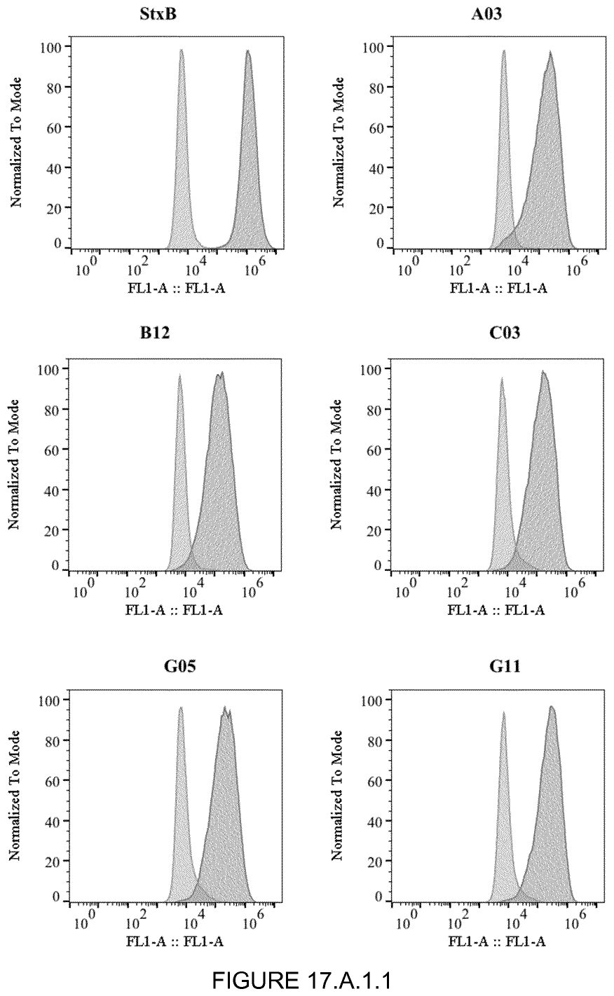

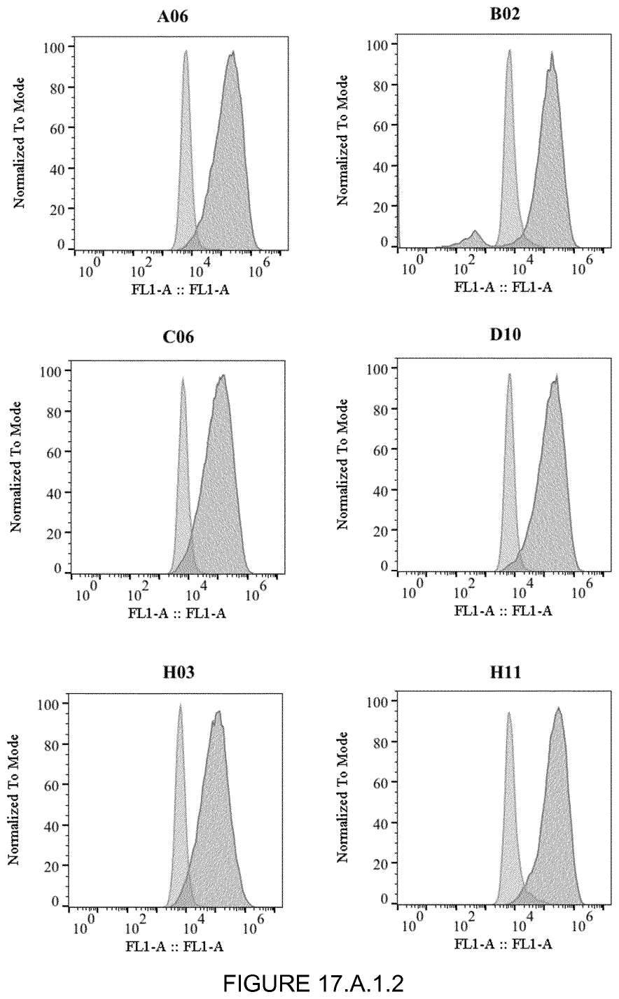

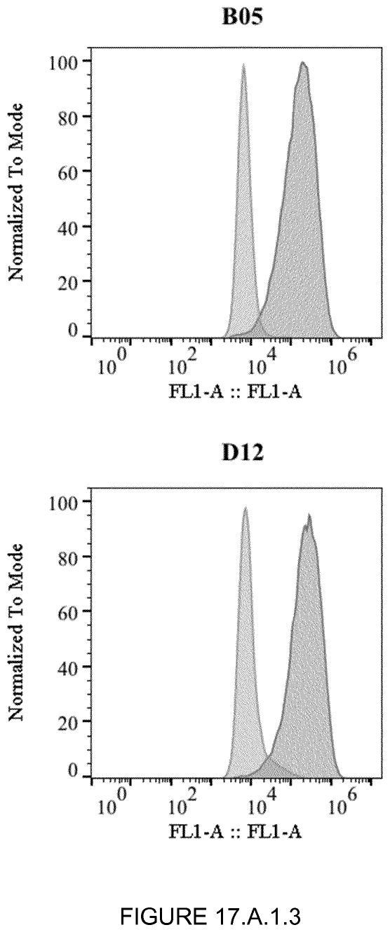

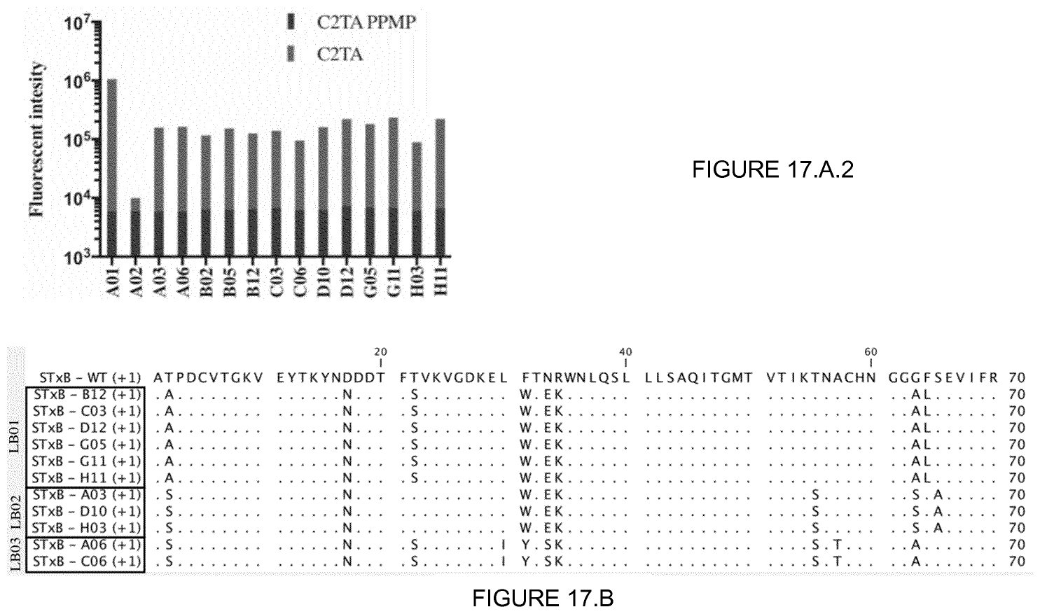

Of the 96 clones, 21 (A03, A06, A08, B02, B05, B12, C02, C03, C06, D04, D07, D10, D12, E7, F12, G05, G11, H03, H07, H11) showed completely “homologous” sequences with wildtype STxB. Of these 21, 13 showed significant GSL-specific binding by FACS (FIG. 17).

Of these 13 clones, 5 unique STxB variant sequences were identified (FIG. 17). (B12, C03, D12, G05, G11, H11)-(A03, D10, H03)-(A06, C06)—were sharing identical sequences, B02 and B05 were unique. These groups will be respectively named LB01, LB02, LB03, LB04 and LB05. To the inventors' knowledge, none of these were previously described in the literature.

The occurrence of each amino acids at each position was analyzed. Interestingly, 8 positions (D17, D18, E28, T31, W34, N35, N55, G60) were never mutated.

Of note, by presenting those phages to Gb3 positive liposomes and after extensive washing, the remaining pulled phages population was highly enriched in relevant phages (efficient selection). It will be appreciated that the skilled person is aware that liposomes containing any commercial glycosphingolipids could be in principle generated and used to screen a library of STxB variants, whose target(s) potentially differ from Gb3, using the protolcol disclosed herein. It is therefore contemplated that the screening method using GSLs presenting magnetic liposomes of the present invention and described herein can be performed, according to particular embodiments:
Through possibly parallel screenings of STxB libraries carried out separately on liposomes batches, each distinct liposome batch specifically presenting one particular purified GSL, or
by performing a screening on liposomes containing a mix of GSLs, in particular a mix of GSLs that do not contain Gb3 to preselect a sub-population of STxB mutants that bind other GSLs.

### Characterization of Gb3 Binders

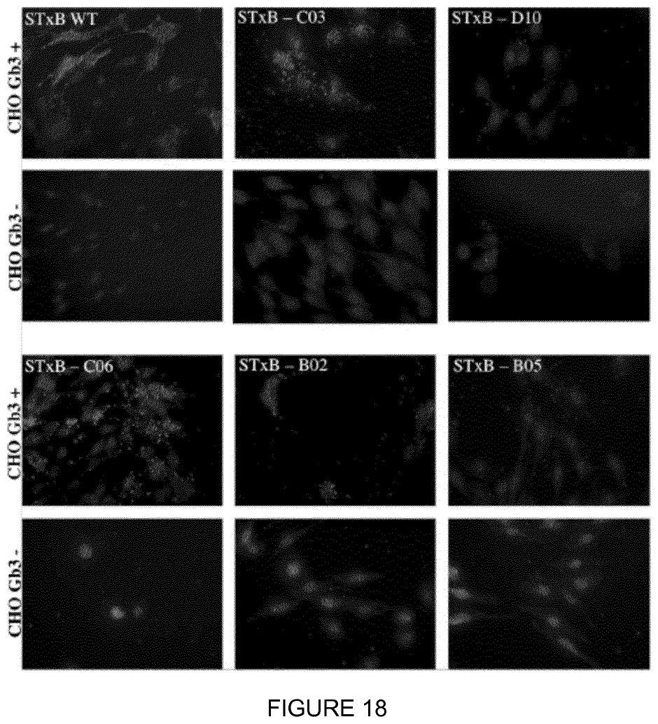

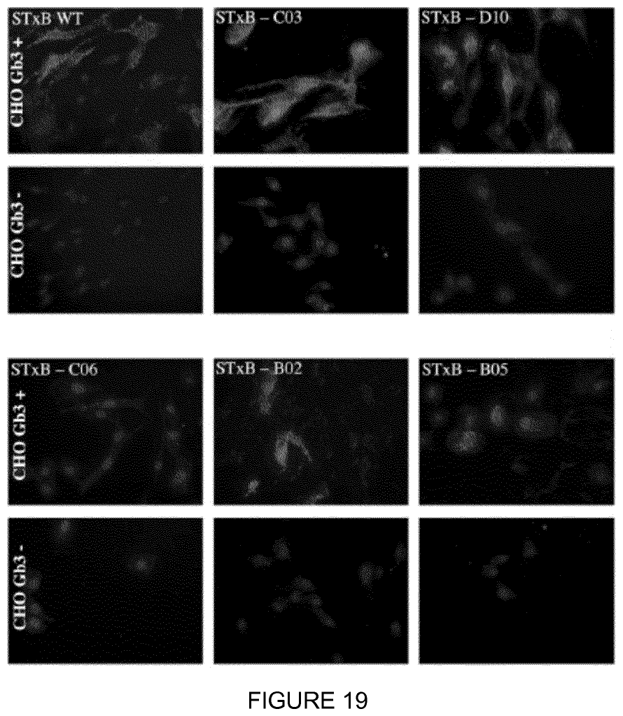

The 5 unique potential Gb3 binders were further characterized by immunofluorescence on CHO cells. Either the phages displaying the STxB variants, or the STxB variants themselves were produced in TG1 bacteria. The proper expression was characterized by immunobloting. Each clone was tested for binding on Gb3+CHO and Gb3-CHO cells. Each of them showed significant binding on Gb3+CHO, and not on Gb3-CHO, whatever they are displayed on the phage (FIG. 18), or free in solution (FIG. 19).

### Ongoing Experiments

### Phage Display Selection of Binders of Other GSLs (for Instance GM3)

The selection is being performed against another GSL than Gb3, as for instance GM3 in order to assess the potential for selecting STxB variants with another binding specificity. Several rounds of selection (from two to five) are performed on magnetic liposomes, where each round consisted in two steps of depletion on GM3-negative liposomes, to remove unspecific binders, followed by 1 step of selection on GM3-positive liposomes (FIG. 10). 10¹² phages are used at each round. The material and methods and protocols described herein apply. A final round of selection could be and has been performed on GM3 positive cells (such as MEB4 cells from mouse melanoma) with two steps of depletion on GM3 negative cells (such as GM95 cells, derived from mouse melanoma and selected for their absence of GM3 expression). Around 100 of clones are picked, sequenced and analyzed for their specific binding to GSLs by flow cytometry on GM3 positive and negative cells.

Accordingly, this can be implemented for a large diversity of GSL as disclosed herein, with the possibility to perform the selection on liposomes, using the purified GSL species, on cells which express the GSL of interest, or also on patient sample taking from biopsy.

1-55. (canceled)

56. A polypeptide having a length from 55 to 85 amino-acid residues:
a) whose polypeptidic sequence comprises or consists essentially of or consists of the consensus sequence: XaPDCVTGKVEYTKYNXbXcXdTFXeVKVGDKXfXgXhXiXjXkXlXmLQSLLLSAQITGMTVTI KXnXoXpCHNXqGXrXsXtEVIFR (SEQ ID NO: 2) where
Xa is selected among: T, A or S, and
Xb, Xc, Xd, Xf, Xm are independently selected among: D, E or N, and
Xe, Xi, Xn, Xp, Xt are independently selected among: T, A or S, and
Xg is selected among: L, I or V, and
Xh is selected among: F, Y, W or A, and
Xj, is selected among: N, E or S, and
Xk is selected among: R, K or E, and
Xl is selected among: W, F, Y or A, and
Xo is selected among: N, E, D or S, and
Xq is selected among: G A or S, and
Xr is selected among: G, A, S or T and
Xs is selected among: F, L or Y,
provided that when Xa is T or A, Xb, Xc, Xd are not D, Xe is not T, Xf is not E, Xg is not L, Xh is not F, Xi is not T, Xj is not N, Xk is not R, Xl is not W, Xm is not N, Xn is not T, Xo is not N, Xp is not A, Xq is not G, Xr is not G, Xs is not F and Xt is not S,
and/or,
b) whose polypeptidic sequence comprises or consists essentially of or consists of the consensus sequence: XaPDCVTGKVEYTKYNXbDDTFXeVKVGDKEXgXhTXjXkWNLQSLLLSAQITGMTVTIKXnNXp CHNGGXrXsXtEVIFR (SEQ ID NO: 37) where Xa, Xb, Xe, Xg, Xh, Xj, Xk, Xn, Xp, Xr, Xs, Xt are as defined in point a),
and/or,
c) whose polypeptidic sequence comprises or consists essentially of a sequence having for structure Xa(S1)XbXcXd(S2)Xe(S3)XfXgXhXiXjXkXlXm(S4)XnXoXp(S5)Xq(S6)XrXsXt(S7) in which S1, S2, S3, S4, S5, S6 and S7, in this order from the N-terminus to the C-terminus of the polypeptide, are defined as follows:
S1 represents the amino-acid sequence PDCVTGKVEYTKYN (SEQ ID NO: 38),
S2 represents the amino-acid sequence TF
S3 represents the amino-acid sequence VKVGDK (SEQ ID NO: 39),
S4 represents the amino-acid sequence LQSLLLSAQITGMTVTIK (SEQ ID NO: 40),
S5 represents the amino-acid sequence CHN
S6 represents amino-acid residue G, and
S7 represents the amino-acid sequence EVIFR (SEQ ID NO: 41), and wherein Xa, Xb, Xc, Xd, Xe, Xf, Xg, Xh, Xi, Xj, Xk, Xl, Xm, Xn, Xo, Xp, Xq, Xr, Xs, Xt are amino-acids as defined in point a) above, and the polypeptidic sequence of the polypeptide keeps at least 80% identity with SEQ ID NO: 1, and/or differs from SEQ ID NO: 1 by one or several conservative amino acid substitution(s),
and/or,
d) whose polypeptidic sequence comprises or consists essentially of or consists of a fragment of contiguous amino-acid residues of at least 55 amino-acid residues, of any one of the sequences defined in a), b) or or c), or comprises or consists essentially of or consists of a portion of any one of the sequences defined in a), b) or c) over a length of at least 55 amino-acid residues;
to the proviso that the polypeptide does not consists of SEQ ID NO: 1, or SEQ D NO: 32, or SEQ ID NO: 36 or SEQ ID NO: 43, or SEQ ID NO: 44, or SEQ ID NO: 45, or SEQ ID NO: 46, or SEQ ID NO: 47, or SEQ ID NO: 48, or SEQ ID NO: 49, or SEQ ID NO: 50, or SEQ ID NO: 51, or SEQ ID NO: 52, or SEQ ID NO: 53,
in particular wherein said polypeptide has the capability, when found under a pentameric form, to bind with at least one glycosphingolipid(s) selected from the group consisting of: Gb3, Gb4, Forsmann like iGb4, fucosyl-GM1, GM1, GM2, GD2, Globo-H, NeuAc-GM3, NeuGc-GM3, GD1a, O-acetyl-GD3, O-acteyl-GD2, O-acetyl-GT3, GD3.

57. The polypeptide of claim 56, which has one or several of the following property(ies) when found under a pentameric form associating five polypeptides as defined in claim 56:
a. the property to bind to a glycosphingolipid selected from the group consisting of: Gb3, Gb4, Forsmann like iGb4, fucosyl-GM1, GM1, GM2, GD2, Globo-H, NeuAc-GM3, NeuGc-GM3, GD1a, O-acetyl-GD3, O-acteyl-GD2, O-acetyl-GT3, GD3, and mixtures thereof and/or
b. an affinity for its target equal or superior to 10²M as measured by ITC and/or
c. an apparent affinity for a membrane displaying its target equal or superior to 10⁶ M⁻¹ as measured by measured by SPR.

58. The polypeptide according to claim 56, which comprises or consists essentially of or consists of any one of the sequence selected among: SEQ ID NO: 3, SEQ ID NO: 5, SEQ ID NO: 7, SEQ ID NO: 9, SEQ ID NO: 11, or fragments thereof.

59. A chimeric protein comprising a polypeptide as defined in claim 56 and a compound fused at one of its end.

60. A pentameric assembly of polypeptides as defined in claim 56.

61. A fusion protein comprising a polypeptide of claim 56 or a polypeptide consisting of SEQ ID NO: 1, or a polypeptide whose polypeptidic sequence comprises or consists essentially of or consists in a sequence having at least 80% identity with SEQ ID NO: 1, and/or differing from SEQ ID NO: 1 by one or several conservative amino acid substitution(s), wherein said polypeptide is fused to a coat protein of a virus or a portion of a coat protein of a virus.

62. The fusion protein according to claim 61, which comprises or consists essentially of or consists of any one of the sequence selected among: SEQ ID NO: 20, SEQ ID NO: 22, SEQ ID NO: 24, SEQ ID NO: 26, SEQ ID NO: 28 and SEQ ID NO: 33 or fragments thereof.

63. A nucleic acid molecule encoding a polypeptide of claim 56 optionally whose nucleotide sequence comprises a stop codon at the end of the sequence encoding the polypeptide according to claim 56 or the polypeptide consisting of SEQ ID NO: 1.

64. A nucleic acid molecule encoding:
a polypeptide of claim 56 optionally whose nucleotide sequence comprises a stop codon at the end of the sequence encoding the polypeptide according to claim 56 or the polypeptide consisting of SEQ ID NO: 1 which is a fusion gene encompassing, from its 3′ to its 5′ extremities:
a. a first nucleic acid sequence encoding polypeptide according to claim 56 or the polypeptide consisting of SEQ ID NO: 1, and
b. a second nucleic acid sequence encoding at least a portion of a pIII filamentous phage coat protein,
wherein said fusion gene comprises between the first and second nucleic sequences at least one stop codon.

65. The nucleic acid molecule according to claim 64, wherein the first nucleic acid sequence comprises or consist essentially of or consists of any one of the sequence selected among: SEQ ID NO: 30, SEQ ID NO: 31, SEQ ID NO: 4, SEQ ID NO: 6, SEQ ID NO: 8, SEQ ID NO: 8 and SEQ ID NO: 12, or comprises or consist essentially of or consists of a nucleic acid sequence having at least 70%, or at least 80%, preferably 85%, more preferably 90% or 95% identity with any one of these sequences and the second nucleic acid sequence comprises or consist essentially of or consists of SEQ ID NO: 19, or a portion of it over a length of 300 bp.

66. The nucleic acid molecule according to claim 64, which consists of any one of SEQ ID NO: 21, SEQ ID NO: 23, SEQ ID NO: 25, SEQ ID NO: 27, SEQ ID NO: 29 and SEQ ID NO: 34, or a variant thereof encoding polypeptides of any one of SEQ ID NO: 20, SEQ ID NO: 22, SEQ ID NO: 24, SEQ ID NO: 26, SEQ ID NO: 28 and SEQ ID NO: 33, respectively, or fragments thereof corresponding to SEQ ID NO: 3, SEQ ID NO: 5, SEQ ID NO: 7, SEQ ID NO: 9, SEQ ID NO: 11 and SEQ ID NO: 32, respectively.

67. A nucleic acid molecule encompassing a nucleic acid molecule as defined in claim 63, which is a vector selected amongst a plasmid, a phagemid and phage vector.

68. The vector of a nucleic acid molecule encoding a polypeptide of claim 56 optionally whose nucleotide sequence comprises a stop codon at the end of the sequence encoding the polypeptide according to claim 56 or the polypeptide consisting of SEQ ID NO: 1, wherein the nucleic acid is a vector selected amongst a plasmid, a phagemid and phage vector, and which is a pHEN2 phagemid comprising a nucleic acid molecule comprising or consisting essentially of or consisting of:
(1) at least one first nucleic acid sequence or a variant thereof, wherein the first nucleic acid molecule encodes:
a polypeptide of claim 56 optionally whose nucleotide sequence comprises a stop codon at the end of the sequence encoding the polypeptide according to claim 56 or the polypeptide consisting of SEQ ID NO: 1 which is a fusion gene encompassing, from its 3′ to its 5′ extremities:
a. a first nucleic acid sequence encoding polypeptide according to claim 56 or the polypeptide consisting of SEQ ID NO: 1, and
b. a second nucleic acid sequence encoding at least a portion of a pIII filamentous phage coat protein,
wherein said fusion gene comprises between the first and second nucleic sequences at least one stop codon, and
wherein the first nucleic acid sequence comprises or consist essentially of or consists of any one of the sequence selected among: SEQ ID NO: 30, SEQ ID NO: 31, SEQ ID NO: 4, SEQ ID NO: 6, SEQ ID NO: 8, SEQ ID NO: 8 and SEQ ID NO: 12, or comprises or consist essentially of or consists of a nucleic acid sequence having at least 70%, or at least 80%, preferably 85%, more preferably 90% or 95% identity with any one of these sequences and the second nucleic acid sequence comprises or consist essentially of or consists of SEQ ID NO: 19, or a portion of it over a length of 300 bp,
(2) at least one stop codon selected among TAG, TAA and TGA, and
(3) a second nucleic acid sequence
in the order of (1), (2) and (3), or a variant thereof, wherein the second nucleic acid molecule encodes:
a polypeptide of claim 56 optionally whose nucleotide sequence comprises a stop codon at the end of the sequence encoding the polypeptide according to claim 56 or the polypeptide consisting of SEQ ID NO: 1 which is a fusion gene encompassing, from its 3′ to its 5′ extremities:
a. a first nucleic acid sequence encoding polypeptide according to claim 56 or the polypeptide consisting of SEQ ID NO: 1, and
b. a second nucleic acid sequence encoding at least a portion of a pIII filamentous phage coat protein,
wherein said fusion gene comprises between the first and second nucleic sequences at least one stop codon, and
wherein the first nucleic acid sequence comprises or consist essentially of or consists of any one of the sequence selected among: SEQ ID NO: 30, SEQ ID NO: 31, SEQ ID NO: 4, SEQ ID NO: 6, SEQ ID NO: 8, SEQ ID NO: 8 and SEQ ID NO: 12, or comprises or consist essentially of or consists of a nucleic acid sequence having at least 70%, or at least 80%, preferably 85%, more preferably 90% or 95% identity with any one of these sequences and the second nucleic acid sequence comprises or consist essentially of or consists of SEQ ID NO: 19, or a portion of it over a length of 300 bp.

69. An expression system comprising:
a) a nucleic acid encoding a polypeptide having a length from 55 to 85 amino-acid residues, whose polypeptidic sequence comprises or consists essentially of or consists of the consensus sequence XaPDCVTGKVEYTKYNXbXcXdTFXeVKVGDKXfXgXhXiXjXkXlXmLQSLLLSAQ ITGMTVTIKXnXoXpCHNXqGXrXsXtEVIFR (SEQ ID NO: 2) where
Xa is selected among: T, A or S, and
Xb, Xc, Xd, Xf, Xm are independently selected among: D, E or N, and
Xe, Xi, Xn, Xp, Xt are independently selected among: T, A or S, and
Xg is selected among: L, I or V, and
Xh is selected among: F, Y, W or A, and
Xj, is selected among: N, E or S, and
Xk is selected among: R, K or E, and
Xl is selected among: W, F, Y or A, and
Xo is selected among: N, E, D or S, and
Xq is selected among: G A or S, and
Xr is selected among: G, A, S or T and
Xs is selected among: F, L or Y,
or,
b) a nucleic acid encoding a polypeptide having a length from to 85 amino-acid residues, whose polypeptidic sequence comprises or consists essentially of or consists in a sequence having at least 75% identity with sequence TPDCVTGKVEYTKYNDDDTFTVKVGDKELFTNRWNLQSLLLSAQITGMTVTIKTNACHNGGGFSE VIFR (SEQ ID NO: 1), and/or differing from SEQ ID NO: 1 by one or several conservative amino acid substitution(s),
or,
c) a nucleic acid encoding a polypeptide having a length from to 85 amino-acid residues, whose polypeptidic sequence comprises or consists essentially of or consists of a fragment of contiguous amino-acid residues of at least 55 amino-acid residues, of any one of the sequences defined in a) or b), or comprises or consists essentially of or consists of a portion of any one of the sequences defined in a) or b) over a length of 55 amino-acid residues,
wherein the expression system is at least one of a plasmid, a phagemid or an expression vector.

70. A host comprising a polypeptide as defined in claim 56.

71. A virus or a library of viruses displaying at its surface a polypeptide as defined in claim 56.

72. A filamentous bacteriophage:
displaying a polypeptide as defined in claim 56, the genome of the filamentous bacteriophage comprising a fusion gene in the form of a nucleic acid molecule encoding a polypeptide of claim 56 optionally whose nucleotide sequence comprises a stop codon at the end of the sequence encoding the polypeptide according to claim 56 or the polypeptide consisting of SEQ ID NO: 1.

73. The filamentous bacteriophage according to claim 72, wherein the displayed STxB-subunit or variant thereof is under the form of one STxB monomer or variant thereof in fusion with a pIII page coat protein, the STxB monomer or variant thereof being assembled with four other free STxB monomers.

74. A bacterial cell comprising a nucleic acid molecule as defined in claim 63.

75. A method of production of filamentous phage(s), or a library thereof, comprising the steps of:
a. Introducing one or several vector(s) as defined in claim 67 into bacterial cell(s), and
b. Culturing the bacterial cell(s) of step (a), optionally in the presence of helper phage(s), and
c. Optionally, recovering the produced filamentous phage(s) or library thereof and/or isolating a particular species of produced filamentous phages or library thereof.

76. In vitro use of a polypeptide as defined in claim 56 for detecting a molecule and/or a cell in a sample.

77. Method for determining the specificity of a virus to glycosphingolipids comprising the following steps:
a) Putting into contact a virus as defined in claim 71 and a support comprising glycosphingolipids on its surface,
b) Incubating the virus and the support comprising glycosphingolipids present on its surface to let the virus bind to the glycosphingolipids,
c) Washing the incubated surface to eliminate the non-bounded virus, and
d) Recovering virus bounded to the glycosphingolipids.

78. A method to identify one or several filamentous phage(s) displaying an STxB-subunit or a variant thereof, which bind to a particular glycosphingolipid or a variant thereof, or to a mix of several glycosphingolipids or variants thereof as a target, said method comprising:
d. Contacting under conditions enabling the binding with the target, a library of filamentous bacteriophages comprising a plurality of filamentous bacteriophages as defined in claim 71, with one or several glycosphingolipid(s) or variant thereof displayed on a support, such as cells expressing one or several glycosphingolipid(s) or variant thereof at their surface, or unilamellar vesicles or liposomes presenting one or several glycosphingolipid(s) or variant thereof, and
e. Separating the filamentous bacteriophages that bind to the target from those that do not bind, for example through washing, and
f. Recovering the filamentous phage(s) bound to the target, and
g. Optionally, analyzing the filamentous phage(s) bound to the target and/or determining the sequence of at least a part of the nucleic acid content of the recovered filamentous phage(s) and/or the sequence of at least a part of the STxB-subunit or a variant thereof displayed by said recovered filamentous phage(s).

79. A method of using a filamentous bacteriophage displaying an STxB-subunit or a variant thereof at its surface as defined in claim 56 to treat one or several neoplasic condition, selected among: ovarian cancer, breast carcinoma, colon cancer, gastric adenocarcinoma, Burkitt's lymphoma, colon carcinoma, melanoma, small cell lung cancer (SCLC), renal carcinoma, neuroblastoma, cervical carcinoma, glioblastoma, renal carcinoma, glioma, retinoblastoma, neuroectodermal cancer, non-small cell lung cancer (NSCLC), Wilms tumor, osteosarcoma, and t-All condition, said method comprising administering to a patient in need thereof a filamentous bacteriophage displaying an STxB-subunit or a variant thereof at its surface as defined in claim claim 56.

80. Use of labelled filamentous bacteriophage displaying an STxB-subunit or a variant thereof at its surface as defined in claim 56, as a probe or marker for in vitro detection of glycosphingolipid(s) or variant(s) thereof.

81. Use of a polypeptide as defined in claim 56 in an in vitro or ex vivo diagnostic method.
